(19) United States
(12) Patent Application Publication
(10) Pub. No.: US 2024/0166738 A1
(43) Pub. Date: May 23, 2024

(54) ANTI-TMEM106B ANTIBODIES FOR TREATING AND PREVENTING CORONAVIRUS INFECTIONS

(71) Applicant: ALECTOR LLC, South San Francisco, CA (US)

(72) Inventors: Herve RHINN, San Francisco, CA (US); Eric BROWN, South San Francisco, CA (US)

(21) Appl. No.: 18/551,963

(22) PCT Filed: Mar. 23, 2022
(86) PCT No.: PCT/US2022/021533

§ 371 Date: Sep. 22, 2023

Related U.S. Application Data
(60) Provisional application No. 63/164,873, filed on Mar. 23, 2021.
(60) Provisional application No. 63/239,498, filed on Sep. 1, 2021.
(60) Provisional application No. 63/318,068, filed on Mar. 9, 2022.

(51) Int. Cl. C07K 16/28; A61P 31/14

(52) U.S. Cl. CPC C07K 16/28; A61P 31/14

(57) ABSTRACT

The present disclosure is generally directed to uses of antibodies that bind to a TMEM106B protein in treating, preventing, or reducing the risk of coronavirus (e.g., SARS-COV-2) infection.

### CROSS-REFERENCE TO RELATED APPLICATIONS

This application claims the priority benefit of U.S. Provisional Application No. 63/164,873, filed Mar. 23, 2021, 63/239,498, filed Sep. 1, 2021, and Ser. No. 63/318,068, filed Mar. 9, 2022, each of which is herein incorporated by reference in their entirety.

### SUBMISSION OF SEQUENCE LISTING ON ASCII TEXT FILE

The content of the following submission of ASCII text file is incorporated herein by reference in its entirety: a computer readable form (CRF) of the Sequence Listing (file name: 4503_015PC03_Seqlisting_ST25.txt: date of creation: Mar. 23, 2022: size: 258,941 bytes).

### FIELD OF THE PRESENT DISCLOSURE

The present disclosure relates to uses of anti-TMEM106B antibodies for treating and preventing coronavirus (e.g., SARS-COV-2) infections.

### BACKGROUND OF THE PRESENT DISCLOSURE

Transmembrane protein 106B (TMEM106B) is a type 2 single pass transmembrane glycoprotein residing primarily within the membrane of late endosome and lysosomes. (See, e.g., Lang et al., 2012, J Biol Chem, 287: 19355-19365; Chen-Plotkin et al., 2012, J Neurosci, 32: 11213-11227; Brady et al., 2013, Human Molecular Genetics, 22:685-695.) TMEM106B is highly conserved in mammals, with the human protein sharing 99% sequence identity with the cynomolgus variant and 97% sequence identify with the murine ortholog.

TMEM106B has a cytoplasmic domain predicted to range from amino acid residues 1-92 (of human TMEM106B: SEQ ID NO: 1), a transmembrane domain predicted to range from amino acid residues 96-117, and a luminal domain predicted to range from amino acid residues 118-274. Five sequence motifs of post-translational N-glycosylation sites (N-X-T/S) span its luminal domain. Simple glycans are added to three of the asparagine residues (N145, N151, and N164) and are not critical for TMEM106B localization. Complex glycans are added to the most C-terminal motifs at N183 and N256: loss of complex glycans on N183 impairs TMEM106B forward transport to endosomes/lysosomes and results in endoplasmic retention. Additionally, N256 complex glycosylation is necessary for proper TMEM106B sorting. (See, e.g., Nicholson and Rademakers, 2016, Acta Neuropathol, 132:639-651.)

The function of TMEM106B has not been fully characterized. Recent reports have indicated a role of TMEM106B in lysosomal function and maintenance by inhibiting trafficking of lysosomes along dendrites. (Sec, e.g., Brady et al., 2013, Human Molecular Genetics, 126:696-698: Schwenk et al., 2014, EMBO J. 33:450-467; Clayton et al., 2018, Brain 141(12): 3428-3442.)

TMEM106B has been identified as a host factor for severe acute respiratory syndrome coronavirus 2 (SARS-COV-2). Genetic deletion of TMEM106B reduced SARS-COV-2 coronavirus replication and infection (Baggen et al, 2021, Nature Genetics, doi.org/10.1038/s41588-021-00805-2; Baggen et al, 2020, bioRxiv, doi: 10.1101/2020.09.28.316281; Wang et al, 2020, bioRxiv, doi: 10.1101/2020/09.24.312298: Wang et al, 2021, Cell, 184:1-14).

There is a need for therapies targeting TMEM106B, including therapeutic antibodies that specifically bind TMEM106B, and/or therapies that are capable of inhibiting the activity of TMEM106B, such as by reducing TMEM106B protein levels or function in order to treat various diseases, disorders, and conditions associated with TMEM106B activity, such as coronavirus infection

All references cited herein, including patent applications and publications, are hereby incorporated by reference in their entirety.

### SUMMARY OF THE PRESENT DISCLOSURE

The present disclosure is generally directed to methods of treating, preventing, or reducing the risk of coronavirus (e.g., SARS-COV-2) infection comprising administering to an individual in need thereof a therapeutically effective amount of an antibody that binds to TMEM106B.

Accordingly, in certain aspects of the present disclosure, the anti-TMEM106 antibody for uses and methods as provided herein has a property selected from the group consisting of: reducing coronavirus replication, reducing coronavirus transmission, reducing coronavirus genome translation, reducing coronavirus cell entry, reducing coronavirus release from an infected cell, and any combination thereof. In some embodiments, the antibody reduces a cytopathic effect in a cell infected with SARS-CoV-2. optionally wherein the cell is a VeroE6 cells or a NCI-H1975 cell.

Other aspects of the present disclosure relate to an isolated (e.g., monoclonal) anti-TMEM106B antibody for use in treating, preventing, or reducing the risk of coronavirus infection, wherein the anti-TMEM106B antibody comprises at least one, two, three, four, five, or six HVRs of an antibody selected from the group consisting of: TM-1, TM-2, TM-3, TM-4, TM-5, TM-6, TM-7, TM-8, TM-9. TM-10, TM-11, TM-12, TM-13, TM-14, TM-15, TM-16, TM-17, TM-18, TM-19, TM-20, TM-21, TM-22, TM-23, TM-24, TM-25, TM-26, TM-27, TM-28, TM-29, TM-30, TM-31, TM-32, TM-33, TM-34, TM-35, TM-37, TM-39, TM-41, TM-42, TM-43, TM-44, TM-45, TM-46, TM-47, TM-48, TM-49, TM-50, TM-51, TM-52, TM-54, TM-56, TM-59, TM-60, TM-61, TM-62, TM-63, TM-64, TM-65, TM-66. TM-67, TM-68, TM-69, TM-70, TM-71, TM-72, TM-73, TM-74, TM-75, TM-76, TM-77, TM-78, TM-79, TM-80, TM-81, TM-82, TM-83, TM-84, TM-85, TM-86, TM-87, TM-88, TM-89, TM90, TM-91, TM-92, TM-93, and TM-94. In some embodiments. the anti-TMEM106B antibody comprises the six HVRs (e.g., as shown in Tables 1-4 below) of the antibody selected from the group consisting of TM-1. TM-2, TM-3, TM-4, TM-5, TM-6, TM-7, TM-8, TM-9, TM-10, TM-11, TM-12, TM-13, TM-14, TM-15. TM-16, TM-17, TM-18, TM-19, TM-20, TM-21, TM-22, TM-23, TM-24, TM-25, TM-26, TM-27, TM-28, TM-29, TM-30, TM-31, TM-32, TM-33, TM-34, TM-35, TM-37, TM-39, TM-41, TM-42, TM-43, TM-44, TM-45, TM-46, TM-47, TM-48, TM-49, TM-50, TM-51, TM-52, TM-54, TM-56, TM-59, TM-60, TM-61, TM-62, TM-63, TM-64, TM-65, TM-66, TM-67, TM-68, TM-69, TM-70, TM-71, TM-72, TM-73, TM-74, TM-75, TM-76, TM-77, TM-78, TM-79, TM-80, TM-81, TM-82, TM-83, TM-84, TM-85, TM-86, TM-87, TM-88, TM-89, TM90, TM-91, TM-92, TM-93. and TM-94.

Other aspects of the present disclosure relate to an isolated (e.g., monoclonal) anti-TMEM106B antibody for use in treating, preventing, or reducing the risk of coronavirus infection which binds essentially the same TMEM106B epitope as a reference anti-TMEM106B antibody comprising the V_{H} and V_{L} (e.g., as shown in Tables 5 and 6 below) of the antibody selected from the group consisting of: TM-1, TM-2, TM-3, TM-4, TM-5, TM-6, TM-7, TM-8, TM-9, TM-10, TM-11, TM-12, TM-13, TM-14, TM-15, TM-16, TM-17, TM-18, TM-19, TM-20, TM-21, TM-22, TM-23, TM-24, TM-25, TM-26, TM-27, TM-28, TM-29, TM-30, TM-31, TM-32, TM-33, TM-34, TM-35, TM-37, TM-39, TM-41, TM-42, TM-43, TM-44, TM-45, TM-46, TM-47, TM-48, TM-49, TM-50, TM-51, TM-52, TM-54, TM-56, TM-59, TM-60, TM-61, TM-62, TM-63, TM-64, TM-65, TM-66, TM-67, TM-68, TM-69, TM-70, TM-71, TM-72, TM-73, TM-74, TM-75, TM-76, TM-77, TM-78, TM-79, TM-80, TM-81, TM-82, TM-83, TM-84, TM-85, TM-86, TM-87, TM-88, TM-89, TM90, TM-91, TM-92, TM-93. and TM-94. Other aspects of the present disclosure relate to an isolated (e.g., monoclonal) anti-TMEM106B antibody for use in treating, preventing, or reducing the risk of coronavirus infection, wherein the antibody competitively inhibits the binding to TMEM106B of an antibody comprising the heavy chain variable region and the light chain variable region of any of the antibodies selected from the group consisting of: TM-1, TM-2. TM-3, TM-4, TM-5, TM-6, TM-7, TM-8, TM-9, TM-10, TM-11, TM-12, TM-13, TM-14, TM-15, TM-16, TM-17, TM-18, TM-19, TM-20, TM-21, TM-22, TM-23, TM-24, TM-25, TM-26, TM-27, TM-28, TM-29, TM-30, TM-31, TM-32, TM-33, TM-34, TM-35, TM-37, TM-39, TM-41, TM-42, TM-43, TM-44, TM-45, TM-46, TM-47, TM-48, TM-49, TM-50, TM-51, TM-52, TM-54, TM-56, TM-59, TM-60, TM-61, TM-62, TM-63, TM-64, TM-65, TM-66, TM-67, TM-68, TM-69, TM-70, TM-71, TM-72, TM-73, TM-74, TM-75, TM-76, TM-77, TM-78, TM-79, TM-80, TM-81, TM-82, TM-83, TM-84, TM-85, TM-86, TM-87, TM-88, TM-89, TM90, TM-91, TM-92, TM-93, and TM-94. In some embodiments, the antibody binds a truncated TMEM106B protein comprising amino acids 122-210 of SEQ ID NO: 1. In some embodiments, the antibody is in Bin 2. In some embodiments, the antibody is in Bin 3. In some embodiments, the antibody is in Bin 4. In some embodiments, the antibody is not in Bin 5. In some embodiments, the antibody is not in Bin 1.

In some embodiments that may be combined with any of the embodiments provided herein, the antibody is a monoclonal antibody. In some embodiments that may be combined with any of the embodiments provided herein, the antibody is of the IgG class, the IgM class, or the IgA class. In some embodiments, the antibody is of the IgG class and has an IgG1, IgG2, or lgG4 isotype. In certain embodiments that may be combined with any of the embodiments provided herein, the anti-TMEM106B antibody is an antibody fragment that binds to an epitope comprising amino acid residues on human TMEM106B or a mammalian TMEM106B protein. In certain embodiments that may be combined with any of the embodiments provided herein, the fragment is a Fab, Fab′, Fab -SH, F(ab′)2, Fv, or scFv fragment. In certain embodiments that may be combined with any of the embodiments provided herein, the anti-TMEM106B antibody is a full-length antibody. In some embodiments that may be combined with any of the embodiments provided herein, the antibody is a humanized antibody or a chimeric antibody.

Other aspects of the present disclosure relate to a pharmaceutical composition comprising an anti-TMEM106B antibody of any of the preceding embodiments, and a pharmaceutically acceptable carrier.

Other aspects of the present disclosure relate to use of an anti-TMEM106B antibody of any of the embodiments herein in the manufacture of a medicament for treating, preventing, or reducing the risk of coronavirus (e.g., SARS-COV-2) infection.

It is to be understood that one, some, or all of the properties of the various embodiments described herein may be combined to form other embodiments of the present invention. These and other aspects of the invention will become apparent to one of skill in the art. These and other embodiments of the invention are further described by the detailed description that follows.

### BRIEF DISCRIPTION OF THE DRAWINGS

FIG. 1 sets forth data showing the effect of anti-TMEM106B antibodies of the present disclosure on cell survival following SARS-COV-2 infection of VeroE6 African green monkey kidney epithelial cells.

FIGS. 2A, 2B, 2C, 2D, 2E, and 2F set forth data showing the effect of anti-TMEM106B antibodies of the present disclosure on cell viability following SARS-COV-2 infection of NCI-H1975 human lung epithelial cells.

FIGS. 3A, 3B, 3C, and 3D set forth data showing a dose tritration effect of anti-TMEM106B antibodies of the present disclosure on cell viability following SARS-COV-2 infection of NCI-H1975 human lung epithelial cells.

### DETAILED DESCRIPTION OF THE PRESENT DISCLOSURE

The present disclosure relates to the use of anti-TMEM106B antibodies (e.g., monoclonal antibodies), and pharmaceutical compositions thereof, for treating, preventing, or reducing the risk of coronavirus infection. In some aspects, methods are provided herein for treating, preventing, or reducing the risk of coronavirus infection by administering to an individual in need thereof an anti-TMEM106B antibody

The techniques and procedures described or referenced herein are generally well understood and commonly employed using conventional methodology by those skilled in the art, such as, for example, the widely utilized methodologies such as those described in Sambrook et al. Molecular Cloning: A Laboratory Manual 3d edition (2001) Cold Spring Harbor Laboratory Press. Cold Spring Harbor, N.Y.: Current Protocols in Molecular Biology (F. M. Ausubel, et al. eds .. (2003); Monoclonal Antibodies: A Practical Approach (P. Shepherd and C. Dean, eds., Oxford University Press, 2000).

### Definitions

The terms “TMEM106B” or “TMEM106B polypeptide” are used interchangeably herein refer herein to any native TMEM106B from any vertebrate source, including mammals such as primates (e.g., humans and cynomolgus monkeys (cynos)) and rodents (e.g., mice and rats), unless otherwise indicated. In some embodiments, the term encompasses both wild-type sequences and naturally occurring variant sequences. e.g., splice variants or allelic variants. In some embodiments, the term encompasses “full-length,” unprocessed TMEM106B as well as any form of TMEM106B that results from processing in the cell. In some embodiments, the TMEM106B is human TMEM106B. In some embodiments, the amino acid sequence of an exemplary TMEM106B is Uniprot Accession No: Q9NUM4 as of Jun. 27, 2006. In some embodiments, the amino acid sequence of an exemplary human TMEM106B is SEQ ID NO: 1.

The terms “anti-TMEM106B antibody.” an “antibody that binds to TMEM106B,” and “antibody that specifically binds TMEM106B” refer to an antibody that is capable of binding TMEM106B with sufficient affinity such that the antibody is useful as a diagnostic and/or therapeutic agent in targeting TMEM106B. In one embodiment, the extent of binding of an anti-TMEM106B antibody to an unrelated. non-TMEM106B polypeptide is less than about 10% of the binding of the antibody to TMEM106B as measured, e.g., by a radioimmunoassay (RIA). In certain embodiments, an antibody that binds to TMEM106B has a dissociation constant (KD) of <1 μM, <100 nM, <10 nM, <1 nM, <0.1 nM, <0.01 nM, or <0.001 nM (e.g., 10⁻⁸ M or less, e.g. from 10⁻⁸ M to 10⁻¹³ M, e.g., from 10⁻⁹ M to 10⁻¹³ M). In certain embodiments, an anti-TMEM106B antibody binds to an epitope of TMEM106B that is conserved among TMEM106B from different species.

With regard to the binding of an antibody to a target molecule, the term “specific binding” or “specifically binds” or is “specific for” a particular polypeptide or an epitope on a particular polypeptide target means binding that is measurably different from a non-specific interaction. Specific binding can be measured, for example, by determining binding of a molecule compared to binding of a control molecule. For example, specific binding can be determined by competition with a control molecule that is similar to the target, for example, an excess of non-labeled target. In this case, specific binding is indicated if the binding of the labeled target to a probe is competitively inhibited by excess unlabeled target. The term “specific binding” or “specifically binds to” or is “specific for” a particular polypeptide or an epitope on a particular polypeptide target as used herein can be exhibited, for example, by a molecule having a KD for the target of about any of 10⁻⁴ M or lower, 10⁻⁵ M or lower, 100 M or lower, 10⁻⁷ M or lower, 108 M or lower, 10⁻⁹ M or lower, 10⁻¹⁰ M or lower, 10⁻¹¹ M or lower, 10⁻¹² M or lower or a KD in the range of 10⁻⁴ M to 10⁻⁶ M or 10⁻⁶ M to 10⁻¹⁰ M or 10⁻⁷ M to 10⁻⁹ M. As will be appreciated by the skilled artisan. affinity and KD values are inversely related. A high affinity for an antigen is measured by a low KD value. In one embodiment, the term “specific binding” refers to binding where a molecule binds to a particular polypeptide or epitope on a particular polypeptide without substantially binding to any other polypeptide or polypeptide epitope.

The term “immunoglobulin” (lg) is used interchangeably with “antibody” herein. The term “antibody” herein is used in the broadest sense and specially covers monoclonal antibodies, polyclonal antibodies, multispecific antibodies (e.g., bispecific antibodies) including those formed from at least two intact antibodies, and antibody fragments so long as they exhibit the desired biological activity.

“Native antibodies” are usually heterotetrameric glycoproteins of about 150.000 Daltons. composed of two identical Light (“L”) chains and two identical heavy (“H”) chains. Each light chain is linked to a heavy chain by one covalent disulfide bond, while the number of disulfide linkages varies among the heavy chains of different immunoglobulin isotypes. Each heavy and light chain also has regularly spaced intra-chain disulfide bridges. Each heavy chain has at one end a variable domain (V_{H}) followed by a number of constant domains. Each light chain has a variable domain at one end (V_{L}) and a constant domain at its other end; the constant domain of the light chain is aligned with the first constant domain of the heavy chain, and the light chain variable domain is aligned with the variable domain of the heavy chain. Particular amino acid residues are believed to form an interface between the light chain and heavy chain variable domains.

For the structure and properties of the different classes of antibodies, see. e.g., Basic and Clinical Immunology, 8th Ed., Daniel P. Stites, Abba I. Terr and Tristram G. Parslow (eds.). Appleton & Lange, Norwalk, CT, 1994. page 71 and Chapter 6.

The light chain from any vertebrate species can be assigned to one of two clearly distinct types, called kappa (“κ”) and lambda (“λ”), based on the amino acid sequences of their constant domains. Depending on the amino acid sequence of the constant domain of their heavy chains (CH). immunoglobulins can be assigned to different classes or isotypes. There are five classes of immunoglobulins: IgA, IgD, IgE, IgG, and IgM, having heavy chains designated alpha (“α”), delta (“δ”). epsilon (“ε”), gamma (“γ”), and mu (“μ”), respectively. The γ and α classes are further divided into subclasses (isotypes) on the basis of relatively minor differences in the CH sequence and function, e.g., humans express the following subclasses: IgG1, IgG2, IgG3, IgG4, IgA1, and IgA2. The subunit structures and three-dimensional configurations of different classes of immunoglobulins are well known and described generally in, for example, Abbas et al., Cellular and Molecular Immunology, 4^{th} ed. (W. B. Saunders Co., 2000).

The “variable region” or “variable domain” of an antibody, such as an anti-TMEM106B antibody of the present disclosure, refers to the amino-terminal domains of the heavy or light chain of the antibody. The variable domains of the heavy chain and light chain may be referred to as “V_{H}” and “V_{L}”, respectively. These domains are generally the most variable parts of the antibody (relative to other antibodies of the same class) and contain the antigen binding sites.

The term “variable” refers to the fact that certain segments of the variable domains differ extensively in sequence among antibodies, such as anti-TMEM106B antibodies of the present disclosure. The variable domain mediates antigen binding and defines the specificity of a particular antibody for its particular antigen. However, the variability is not evenly distributed across the entire span of the variable domains. Instead, it is concentrated in three segments called hypervariable regions (HVRs) both in the light-chain and the heavy chain variable domains. The more highly conserved portions of variable domains are called the framework regions (FR). The variable domains of native heavy and light chains cach comprise four FR regions, largely adopting a beta-sheet configuration, connected by three HVRs. which form loops connecting, and in some cases forming part of, the beta-sheet structure. The HVRs in each chain are held together in close proximity by the FR regions and, with the HVRs from the other chain, contribute to the formation of the antigen-binding site of antibodies (see Kabat et al., Sequences of Immunological Interest, Fifth Edition. National Institute of Health. Bethesda. MD (1991)). The constant domains are not involved directly in the binding of antibody to an antigen, but exhibit various effector functions, such as participation of the antibody in antibody-dependent-cellular toxicity.

The term “monoclonal antibody” as used herein refers to an antibody, such as a monoclonal anti-TMEM106B antibody of the present disclosure, obtained from a population of substantially homogeneous antibodies. i.e., the individual antibodies comprising the population are identical except for possible naturally occurring mutations and/or post-translation modifications (e.g., isomerizations, amidations, etc.) that may be present in minor amounts. Monoclonal antibodies are highly specific, being directed against a single antigenic site. In contrast to polyclonal antibody preparations which typically include different antibodies directed against different determinants (epitopes), cach monoclonal antibody is directed against a single determinant on the antigen. In addition to their specificity, the monoclonal antibodies are advantageous in that they are synthesized by the hybridoma culture, uncontaminated by other immunoglobulins. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by a variety of techniques, including, for example, the hybridoma method, recombinant DNA methods, and technologies for producing human or human-like antibodies in animals that have parts or all of the human immunoglobulin loci or genes encoding human immunoglobulin sequences.

The terms “full-length antibody,” “intact antibody” or “whole antibody” are used interchangeably to refer to an antibody, such as an anti-TMEM106B antibody of the present disclosure, in its substantially intact form, as opposed to an antibody fragment. Specifically, whole antibodies include those with heavy and light chains including an Fc region. The constant domains may be native sequence constant domains (e.g., human native sequence constant domains) or amino acid sequence variants thereof. In some cases, the intact antibody may have one or more effector functions.

An “antibody fragment” refers to a molecule other than an intact antibody that comprises a portion of an intact antibody that binds the antigen to which the intact antibody binds. Examples of antibody fragments include Fab, Fab′, F(ab′)₂ and Fv fragments; diabodies; linear antibodies (see U.S. Pat. No. 5,641,870, Example 2; Zapata et al., Protein Eng. 8(10):1057-1062 (1995)); single-chain antibody molecules and multispecific antibodies formed from antibody fragments.

Papain digestion of antibodies, such as anti-TMEM106B antibodies of the present disclosure, produces two identical antigen-binding fragments, called “Fab” fragments, and a residual “Fc” fragment, a designation reflecting the ability to crystallize readily. The Fab fragment consists of an entire light chain along with the variable region domain of the heavy chain (V_{H}), and the first constant domain of one heavy chain (C_{H}1). Each Fab fragment is monovalent with respect to antigen binding, i.e., it has a single antigen-binding site. Pepsin treatment of an antibody yields a single large F(ab′)₂ fragment which roughly corresponds to two disulfide linked Fab fragments having different antigen-binding activity and is still capable of cross-linking antigen. Fab′ fragments differ from Fab fragments by having a few additional residues at the carboxy terminus of the C_{H}1 domain including one or more cysteines from the antibody hinge region. Fab′-SH is the designation herein for Fab′ in which the cysteine residue(s) of the constant domains bear a free thiol group. F(ab′)₂ antibody fragments originally were produced as pairs of Fab′ fragments which have hinge cysteines between them. Other chemical couplings of antibody fragments are also known.

The Fc fragment comprises the carboxy-terminal portions of both heavy chains held together by disulfides. The effector functions of antibodies are determined by sequences in the Fc region, the region which is also recognized by Fc receptors (FcR) found on certain types of cells.

“Functional fragments” of antibodies, such as anti-TMEM106B antibodies of the present disclosure, comprise a portion of an intact antibody, generally including the antigen binding or variable region of the intact antibody or the Fc region of an antibody which retains or has modified FcR binding capability. Examples of antibody fragments include linear antibody, single-chain antibody molecules and multispecific antibodies formed from antibody fragments.

The term “diabodies” refers to small antibody fragments prepared by constructing sFv fragments (see preceding paragraph) with short linkers (about 5-10) residues) between the V_{H} and V_{L} domains such that inter-chain but not intra-chain pairing of the variable domains is achieved, thereby resulting in a bivalent fragment, i.e., a fragment having two antigen-binding sites. Bispecific diabodies are heterodimers of two “crossover” sFv fragments in which the V_{H} and V_{L} domains of the two antibodies are present on different polypeptide chains.

As used herein, a “chimeric antibody” refers to an antibody (immunoglobulin), such as a chimeric anti-TMEM106B antibody of the present disclosure, in which a portion of the heavy and/or light chain is identical with or homologous to corresponding sequences in antibodies derived from a particular species or belonging to a particular antibody class or subclass, while the remainder of the chain(s) is(are) identical with or homologous to corresponding sequences in antibodies derived from another species or belonging to another antibody class or subclass, as well as fragments of such antibodies, so long as they exhibit the desired biological activity. Chimeric antibodies of interest herein include PRIMATIZED® antibodies wherein the antigen-binding region of the antibody is derived from an antibody produced by, e.g., immunizing macaque monkeys with an antigen of interest. As used herein, “humanized antibody” is used a subset of “chimeric antibodies.”

“Humanized” forms of non-human (e.g., murine) antibodies, such as humanized forms of anti-TMEM106B antibodies of the present disclosure, are chimeric antibodies comprising amino acid residues from non-human HVRs and amino acid residues from human FRs. In certain embodiments, a humanized antibody will comprise substantially all of at least one, and typically two, variable domains, in which all or substantially all of the HVRs (e.g., CDRs) correspond to those of a non-human antibody, and all or substantially all of the FRs correspond to those of a human antibody. A humanized antibody optionally may comprise at least a portion of an antibody constant region derived from a human antibody. A “humanized form” of an antibody, e.g., a non-human antibody, refers to an antibody that has undergone humanization.

A “human antibody” is one that possesses an amino-acid sequence corresponding to that of an antibody, such as an anti-TMEM106B antibody of the present disclosure, produced by a human and/or has been made using any of the techniques for making human antibodies as disclosed herein. This definition of a human antibody specifically excludes a humanized antibody comprising non-human antigen-binding residues. Human antibodies can be produced using various techniques known in the art, including phage-display libraries and yeast-display libraries. Human antibodies can be prepared by administering the antigen to a transgenic animal that has been modified to produce such antibodies in response to antigenic challenge, but whose endogenous loci have been disabled, e.g., immunized xenomice as well as generated via a human B-cell hybridoma technology.

The term “hypervariable region,” “HVR,” or “HV,” when used herein refers to the regions of an antibody-variable domain, such as that of an anti-TMEM106B antibody of the present disclosure, that are hypervariable in sequence and/or form structurally defined loops. Generally, antibodies comprise six HVRs; three in the V_{H} (H1, H2, H3), and three in the V_{L} (L1, L2, L3). In native antibodies, H3 and L3 display the most diversity of the six HVRs, and H3 in particular is believed to play a unique role in conferring fine specificity to antibodies. Naturally occurring camelid antibodies consisting of a heavy chain only are functional and stable in the absence of light chain.

A number of HVR delineations are in use and are encompassed herein. In some embodiments, the HVRs may be Kabat complementarity-determining regions (CDRs) based on sequence variability and are the most commonly used (Kabat et al., supra). In some embodiments, the HVRs may be Chothia CDRs. Chothia refers instead to the location of the structural loops (Chothia and Lesk J. Mol. Biol. 196:901-917 (1987)). In some embodiments, the HVRs may be AbM HVRs. The AbM HVRs represent a compromise between the Kabat CDRs and Chothia structural loops, and are used by Oxford Molecular's AbM antibody-modeling software. In some embodiments, the HVRs may be “contact” HVRs. The “contact” HVRs are based on an analysis of the available complex crystal structures. The residues from each of these HVRs are noted below.

| Loop | Kabat | AbM | Chothia | Contact |
|---|---|---|---|---|
| L1 | L24-L34 | L24-L34 | L26-L32 | L30-L36 |
| L2 | L50-L56 | L50-L56 | L50-L52 | L46-L55 |
| L3 | L89-L97 | L89-L97 | L91-L96 | L89-L96 |
| H1 | H31-H35B | H26-H35B | H26-H32 | H30-H35B (Kabat |
| | | | | numbering) |
| H1 | H31-H35 | H26-H35 | H26-H32 | H30-H35 (Chothia |
| | | | | numbering) |
| H2 | H50-H65 | H50-H58 | H53-H55 | H47-H58 |
| H3 | H95-H102 | H95-H102 | H96-H101 | H93-H101 |

HVRs may comprise “extended HVRs” as follows: 24-36 or 24-34 (L1), 46-56 or 50-56 (L2), and 89-97 or 89-96 (L3) in the VL, and 26-35 (H1), 50-65 or 49-65 (a preferred embodiment) (H2), and 93-102, 94-102, or 95-102 (H3) in the VH. The variable-domain residues are numbered according to Kabat et al., supra, for each of these extended-HVR definitions.

“Framework” or “FR” residues are those variable-domain residues other than the HVR residues as herein defined.

An “acceptor human framework” as used herein is a framework comprising the amino acid sequence of a V_{L} or V_{H} framework derived from a human immunoglobulin framework or a human consensus framework. An acceptor human framework “derived from” a human immunoglobulin framework or a human consensus framework may comprise the same amino acid sequence thereof, or it may comprise pre-existing amino acid sequence changes. In some embodiments, the number of pre-existing amino acid changes are 10 or less, 9 or less, 8 or less, 7 or less, 6 or less, 5 or less, 4 or less, 3 or less, or 2 or less. Where pre-existing amino acid changes are present in a VH, preferable those changes occur at only three, two, or one of positions 71H, 73H and 78H; for instance, the amino acid residues at those positions may by 71A, 73T and/or 78A. In one embodiment, the V_{L} acceptor human framework is identical in sequence to the V_{L} human immunoglobulin framework sequence or human consensus framework sequence.

A “human consensus framework” is a framework that represents the most commonly occurring amino acid residues in a selection of human immunoglobulin V_{L} or V_{H} framework sequences. Generally, the selection of human immunoglobulin V_{L} or V_{H} sequences is from a subgroup of variable domain sequences. Generally, the subgroup of sequences is a subgroup as in Kabat et al., Sequences of Proteins of Immunological Interest, 5th Ed. Public Health Service, National Institutes of Health, Bethesda, MD (1991). Examples include for the VL, the subgroup may be subgroup kappa I, kappa II, kappa III or kappa IV as in Kabat et al., supra. Additionally, for the VH, the subgroup may be subgroup I, subgroup II, or subgroup III as in Kabat et al., supra.

An “amino-acid modification” at a specified position, e.g., of an anti-TMEM106B antibody of the present disclosure, refers to the substitution or deletion of the specified residue, or the insertion of at least one amino acid residue adjacent the specified residue. Insertion “adjacent” to a specified residue means insertion within one to two residues thereof The insertion may be N-terminal or C-terminal to the specified residue. The preferred amino acid modification herein is a substitution.

An “affinity-matured” antibody, such as an affinity matured anti-TMEM106B antibody of the present disclosure, is one with one or more alterations in one or more I-WRs thereof that result in an improvement in the affinity of the antibody for antigen, compared to a parent antibody that does not possess those alteration(s). In one embodiment, an affinity-matured antibody has nanomolar or even picomolar affinities for the target antigen. Affinity-matured antibodies are produced by procedures known in the art. For example, Marks et al. Bio/Technology 10:779-783 (1992) describes affinity maturation by V_{H}- and V_{L}-domain shuffling. Random mutagenesis of HVR and/or framework residues is described by, for example: Barbas et al. Proc Nat. Acad. Sci. USA 91:3809-3813 (1994); Schier et al. Gene 169:147-155 (1995); Yelton et al. J. Immunol. 155: 1994-2004 (1995); Jackson et al. J. Immunol. 154(7):3310-9 (1995); and Hawkins et al., J. Mol. Biol. 226:889-896 (1992).

“Fv” is the minimum antibody fragment which comprises a complete antigen-recognition and -binding site. This fragment consists of a dimer of one heavy- and one light-chain variable region domain in tight, non-covalent association. From the folding of these two domains emanate six hypervariable loops (3 loops each from the H and L chain) that contribute the amino acid residues for antigen binding and confer antigen binding specificity to the antibody. However, even a single variable domain (or half of an Fv comprising only three HVRs specific for an antigen) has the ability to recognize and bind antigen, although at a lower affinity than the entire binding site.

“Single-chain Fv” also abbreviated as “sFv” or “scFv” are antibody fragments that comprise the V_{H} and V_{L} antibody domains connected into a single polypeptide chain. Preferably, the sFv polypeptide further comprises a polypeptide linker between the V_{H} and V_{L} domains, which enables the sFv to form the desired structure for antigen binding.

Antibody “effector functions” refer to those biological activities attributable to the Fc region (a native sequence Fc region or amino acid sequence variant Fc region) of an antibody, and vary with the antibody isotype.

The term “Fc region” herein is used to define a C-terminal region of an immunoglobulin heavy chain, including native-sequence Fc regions and variant Fc regions. Although the boundaries of the Fc region of an immunoglobulin heavy chain might vary, the human IgG heavy-chain Fc region is usually defined to stretch from an amino acid residue at position Cys226, or from Pro230, to the carboxyl-terminus thereof. The C-terminal lysine (residue 447 according to the EU numbering system) of the Fc region may be removed, for example, during production or purification of the antibody, or by recombinantly engineering the nucleic acid encoding a heavy chain of the antibody. Accordingly, a composition of intact antibodies may comprise antibody populations with all K447 residues removed, antibody populations with no K447 residues removed, and antibody populations having a mixture of antibodies with and without the K447 residue. Suitable native-sequence Fc regions for use in the antibodies of the present disclosure include human IgG1, IgG2, IgG3 and IgG4.

A “native sequence Fc region” comprises an amino acid sequence identical to the amino acid sequence of an Fc region found in nature. Native sequence human Fc regions include a native sequence human IgG1 Fc region (non-A and A allotypes); native sequence human IgG2 Fc region; native sequence human IgG3 Fc region; and native sequence human IgG4 Fc region as well as naturally occurring variants thereof

A “variant Fc region” comprises an amino acid sequence which differs from that of a native sequence Fc region by virtue of at least one amino acid modification, preferably one or more amino acid substitution(s). Preferably, the variant Fc region has at least one amino acid substitution compared to a native sequence Fc region or to the Fc region of a parent polypeptide, e.g. from about one to about ten amino acid substitutions, and preferably from about one to about five amino acid substitutions in a native sequence Fc region or in the Fc region of the parent polypeptide. The variant Fc region herein will preferably possess at least about 80% homology with a native sequence Fc region and/or with an Fc region of a parent polypeptide, and most preferably at least about 90% homology therewith, more preferably at least about 95% homology therewith.

“Fc receptor” or “FcR” describes a receptor that binds to the Fc region of an antibody. The preferred FcR is a native sequence human FcR. Moreover, a preferred FcR is one which binds an IgG antibody (a gamma receptor) and includes receptors of the FcγRI, FcγRII, and FcγRIII subclasses, including allelic variants and alternatively spliced forms of these receptors, FcγRII receptors include FcγRIIA (an “activating receptor”) and FcγRIIB (an “inhibiting receptor”), which have similar amino acid sequences that differ primarily in the cytoplasmic domains thereof. Activating receptor FcγRIIA contains an immunoreceptor tyrosine-based activation motif (“ITAM”) in its cytoplasmic domain. Inhibiting receptor FcγRIIB contains an immunoreceptor tyrosine-based inhibition motif (“ITIM”) in its cytoplasmic domain. Other FcRs, including those to be identified in the future, are encompassed by the term “FcR” herein. FcRs can also increase the serum half-life of antibodies.

As used herein, “percent (%) amino acid sequence identity” and “homology” with respect to a peptide, polypeptide or antibody sequence refers to the percentage of amino acid residues in a candidate sequence that are identical with the amino acid residues in the specific peptide or polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or MEGALIGN™ (DNASTAR) software. Those skilled in the art can determine appropriate parameters for measuring alignment, including any algorithms known in the art needed to achieve maximal alignment over the full-length of the sequences being compared.

The term “compete” when used in the context of antibodies (e.g., neutralizing antibodies) that compete for the same epitope means competition between antibody as determined by an assay in which the antibody being tested prevents or inhibits (e.g., reduces) specific binding of a reference molecule (e.g., a ligand, or a reference antibody) to a common antigen (e.g., TMEM106B or a fragment thereof). Numerous types of competitive binding assays can be used to determine if antibody competes with another, for example: solid phase direct or indirect radioimmunoassay (RIA), solid phase direct or indirect enzyme immunoassay (EIA), sandwich competition assay (see, e.g., Stahli et al., 1983, Methods in Enzymology 9:242-253); solid phase direct biotin-avidin EIA (see, e.g., Kirkland et al., 1986, J. Immunol. 137:3614-3619) solid phase direct labeled assay, solid phase direct labeled sandwich assay (see, e.g., Harlow and Lane, 1988, Antibodies, A Laboratory Manual, Cold Spring Harbor Press); solid phase direct label RIA using 1-125 label (see, e.g., Morel et al., 1988, Molec. Immunol. 25:7-15); solid phase direct biotin-avidin EIA (see, e.g., Cheung, et al., 1990, Virology 176:546-552); and direct labeled RIA (Moldenhauer et al., 1990, Scand. J. Immunol. 32:77-82). Typically, such an assay involves the use of purified antigen bound to a solid surface or cells bearing either of these, an unlabelled test antibody and a labeled reference antibody. Competitive inhibition is measured by determining the amount of label bound to the solid surface or cells in the presence of the test antibody. Usually the test antibody is present in excess. Antibodies identified by competition assay (competing antibodies) include antibodies binding to the same epitope as the reference antibody and antibodies binding to an adjacent epitope sufficiently proximal to the epitope bound by the reference antibody for steric hindrance to occur. Additional details regarding methods for determining competitive binding are provided below and, in the examples, herein. Usually, when a competing antibody is present in excess, it will inhibit (e.g., reduce) specific binding of a reference antibody to a common antigen by at least 20%, 30%, 40%, 50%, 60%, 70%, 80%, 85%, 90%, 95%, 97.5%, and/or near 100%.

Antibodies that compete for binding to the same region of TMEM106B are considered to be in the same antibody “bin.” Exemplary bins of antibodies that bind to TMEMO6B are discussed herein, e.g., in Examples 4 and 6.

As used herein, an antibody in “Bin 1” refers to an antibody that competes for binding to the same or overlapping TMEM106B region as the TM1, TM17, TM22, TM26, TM79, and/or TM82 antibodies provided herein.

As used herein, an antibody in “Bin 2” refers to an antibody that competes for binding to the same or overlapping TMEM106B region as the TM3, TM9, TM10, TM11, TM12, TM13, TM18, TM19, TM21, TM24, TM25, TM32, TM35, TM37, TM42, TM45, TM48, TM54, TM59, TM60, TM61, and/or TM76 antibodies provided herein.

As used herein, an antibody in “Bin 3” refers to an antibody that competes for binding to the same or overlapping TMEM106B region as the TM7, TM15, TM83, and/or TM84 antibodies provided herein.

As used herein, an antibody in “Bin 4” refers to an antibody that competes for binding to the same or overlapping TMEM106B region as the TMS, TM28, TM29, TM30, TM63, TM64, TM72, TM78, TM80, TM86, and/or TM88 antibodies provided herein.

As used herein, an antibody in “Bin 5” refers to an antibody that does not bind to a truncated TMEM106B comprising amino acids 122-210 of SEQ ID NO:1.

As used herein, an “interaction” between a TMEM106B polypeptide and a second polypeptide encompasses, without limitation, protein-protein interaction, a physical interaction, a chemical interaction, binding, covalent binding, and ionic binding As used herein, an antibody “inhibits interaction” between two polypeptides when the antibody disrupts, reduces, or completely eliminates an interaction between the two polypeptides. An antibody of the present disclosure, thereof, “inhibits interaction” between two polypeptides when the antibody thereof binds to one of the two polypeptides. In some embodiments, the interaction can be inhibited by at least about any of 20%, 30%, 40%, 50%, 60%, 70%, 80%, 85%, 90%, 95%, 97.5%, and/or near 100%.

The term “epitope” includes any determinant capable of being bound by an antibody. An epitope is a region of an antigen that is bound by an antibody that targets that antigen, and when the antigen is a polypeptide, includes specific amino acids that directly contact the antibody. Most often, epitopes reside on polypeptides, but in some instances, can reside on other kinds of molecules, such as nucleic acids. Epitope determinants can include chemically active surface groupings of molecules such as amino acids, sugar side chains, phosphoryl or sulfonyl groups, and can have specific three-dimensional structural characteristics, and/or specific charge characteristics. Generally, antibodies specific for a particular target antigen will preferentially recognize an epitope on the target antigen in a complex mixture of polypeptides and/or macromolecules.

An “agonist” antibody or an “activating” antibody is an antibody that induces (e.g., increases) one or more activities or functions of the antigen after the antibody binds the antigen.

An “antagonist” antibody or a “blocking” antibody or an “inhibitory” antibody is an antibody that reduces, inhibits, and/or eliminates (e.g., decreases) antigen binding to one or more ligand after the antibody binds the antigen, and/or that reduces, inhibits, and/or eliminates (e.g., decreases) one or more activities or functions of the antigen after the antibody binds the antigen. In some embodiments, antagonist antibodies, or blocking antibodies, or inhibitory antibodies substantially or completely inhibit antigen binding to one or more ligand and/or one or more activities or functions of the antigen.

An “isolated” antibody, such as an isolated anti-TMEM106B antibody of the present disclosure, is one that has been identified, separated and/or recovered from a component of its production environment (e.g., naturally or recombinantly). Preferably, the isolated antibody is free of association with all other contaminant components from its production environment. Contaminant components from its production environment, such as those resulting from recombinant transfected cells, are materials that would typically interfere with research, diagnostic or therapeutic uses for the antibody, and may include enzymes, hormones, and other proteinaceous or non-proteinaceous solutes. In preferred embodiments, the antibody will be purified: (1) to greater than 95% by weight of antibody as determined by, for example, the Lowry method, and in some embodiments, to greater than 99% by weight; (2) to a degree sufficient to obtain at least 15 residues of N-terminal or internal amino acid sequence by use of a spinning cup sequenator, or (3) to homogeneity by SDS-PAGE under non-reducing or reducing conditions using Coomassie blue or, preferably, silver stain. Isolated antibody includes the antibody in situ within recombinant T-cells since at least one component of the antibody's natural environment will not be present. Ordinarily, however, an isolated polypeptide or antibody will be prepared by at least one purification step.

An “isolated” nucleic acid molecule encoding an antibody, such as an anti-TMEM106B antibody of the present disclosure, is a nucleic acid molecule that is identified and separated from at least one contaminant nucleic acid molecule with which it is ordinarily associated in the environment in which it was produced. Preferably, the isolated nucleic acid is free of association with all components associated with the production environment. The isolated nucleic acid molecules encoding the polypeptides and antibodies herein is in a form other than in the form or setting in which it is found in nature. Isolated nucleic acid molecules therefore are distinguished from nucleic acid encoding the polypeptides and antibodies herein existing naturally in cells.

The term “vector,” as used herein, is intended to refer to a nucleic acid molecule capable of transporting another nucleic acid to which it has been linked. One type of vector is a “plasmid,” which refers to a circular double stranded DNA into which additional DNA segments may be ligated. Another type of vector is a phage vector. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors,” or simply, “expression vectors.” In general, expression vectors of utility in recombinant DNA techniques are often in the form of plasmids. In the present specification, “plasmid” and “vector” may be used interchangeably as the plasmid is the most commonly used form of vector.

“Polynucleotide,” or “nucleic acid,” as used interchangeably herein, refer to polymers of nucleotides of any length, and include DNA and RNA. The nucleotides can be deoxyribonucleotides, ribonucleotides, modified nucleotides or bases, and/or their analogs, or any substrate that can be incorporated into a polymer by DNA or RNA polymerase or by a synthetic reaction.

A “host cell” includes an individual cell or cell culture that can be or has been a recipient for vector(s) for incorporation of polynucleotide inserts. Host cells include progeny of a single host cell, and the progeny may not necessarily be completely identical (in morphology or in genomic DNA complement) to the original parent cell due to natural, accidental, or deliberate mutation. A host cell includes cells transfected in vivo with a polynucleotide(s) of this invention.

“Carriers” as used herein include pharmaceutically acceptable carriers, excipients, or stabilizers that are nontoxic to the cell or mammal being exposed thereto at the dosages and concentrations employed.

As used herein, the term “preventing” includes providing prophylaxis with respect to occurrence or recurrence of a particular disease, disorder, or condition in an individual. An individual may be predisposed to, susceptible to a particular disease, disorder, or condition, or at risk of developing such a disease, disorder, or condition, but has not yet been diagnosed with the disease, disorder, or condition.

As used herein, an individual “at risk” of developing a particular disease, disorder, or condition may or may not have detectable disease or symptoms of disease, and may or may not have displayed detectable disease or symptoms of disease prior to the treatment methods described herein. “At risk” denotes that an individual has one or more risk factors, which are measurable parameters that correlate with development of a particular disease, disorder, or condition, as known in the art. An individual having one or more of these risk factors has a higher probability of developing a particular disease, disorder, or condition than an individual without one or more of these risk factors.

As used herein, the term “treatment” refers to clinical intervention designed to alter the natural course of the individual being treated during the course of clinical pathology. Desirable effects of treatment include decreasing the rate of progression, ameliorating or palliating the pathological state, and remission or improved prognosis of a particular disease, disorder, or condition. An individual is successfully “treated”, for example, if one or more symptoms associated with a particular disease, disorder, or condition are mitigated or eliminated.

An “effective amount” refers to at least an amount effective, at dosages and for periods of time necessary, to achieve the desired therapeutic or prophylactic result. An effective amount can be provided in one or more administrations. An effective amount herein may vary according to factors such as the disease state, age, sex, and weight of the individual, and the ability of the treatment to elicit a desired response in the individual. An effective amount is also one in which any toxic or detrimental effects of the treatment are outweighed by the therapeutically beneficial effects. For prophylactic use, beneficial or desired results include results such as eliminating or reducing the risk, lessening the severity, or delaying the onset of the disease, including biochemical, histological and/or behavioral symptoms of the disease, its complications and intermediate pathological phenotypes presenting during development of the disease. For therapeutic use, beneficial or desired results include clinical results such as decreasing one or more symptoms resulting from the disease, increasing the quality of life of those suffering from the disease, decreasing the dose of other medications required to treat the disease, enhancing effect of another medication such as via targeting, delaying the progression of the disease, and/or prolonging survival. An effective amount of drug, compound, or pharmaceutical composition is an amount sufficient to accomplish prophylactic or therapeutic treatment either directly or indirectly. As is understood in the clinical context, an effective amount of a drug, compound, or pharmaceutical composition may or may not be achieved in conjunction with another drug, compound, or pharmaceutical composition. Thus, an “effective amount” may be considered in the context of administering one or more therapeutic agents, and a single agent may be considered to be given in an effective amount if, in conjunction with one or more other agents, a desirable result may be or is achieved.

An “individual” for purposes of treatment, prevention, or reduction of risk refers to any animal classified as a mammal, including humans, domestic and farm animals, and zoo, sport, or pet animals, such as dogs, horses, rabbits, cattle, pigs, hamsters, gerbils, mice, ferrets, rats, cats, and the like. In some embodiments, the individual is human.

As used herein, administration “in conjunction” with another compound or composition includes simultaneous administration and/or administration at different times. Administration in conjunction also encompasses administration as a co-formulation or administration as separate compositions, including at different dosing frequencies or intervals, and using the same route of administration or different routes of administration. In some embodiments, administration in conjunction is administration as a part of the same treatment regimen.

The term “about” as used herein refers to the usual error range for the respective value readily known to the skilled person in this technical field. Reference to “about” a value or parameter herein includes (and describes) embodiments that are directed to that value or parameter per se.

As used herein and in the appended claims, the singular forms “a,” “an,” and “the” include plural reference unless the context clearly indicates otherwise. For example, reference to an “antibody” is a reference to from one to many antibodies, such as molar amounts, and includes equivalents thereof known to those skilled in the art, and so forth.

As used herein a “TMEM106B” protein of the present disclosure includes, without limitation, a mammalian TMEM106B protein, human TMEM106B protein, primate TMEM106B protein, cynomolgus (cyno) TMEM106B protein, mouse TMEM106B protein, and rat TMEM106B protein. Additionally, anti-TMEM106B antibodies of the present disclosure may bind an epitope within one or more of a mammalian TMEM106B protein, human TMEM106B protein, primate TMEM106B, cyno TMEM106B protein, mouse TMEM106B protein, and rat TMEM106B protein.

It is understood that aspect and embodiments of the present disclosure described herein include “comprising,” “consisting,” and “consisting essentially of” aspects and embodiments.

### TMEM106B Protein

Provided herein are methods of treating, preventing, or reducing risk of coronavirus infection by administering an anti-TMEM106B antibody to an individual in need thereof.

In one aspect, the present disclosure provides isolated (e.g., monoclonal) antibodies for use in treating, preventing, or reducing the risk of coronavirus infection, wherein the antibodies bind to an epitope within a TMEM106B protein of the present disclosure. TMEM106B proteins of the present disclosure include, without limitation, a mammalian TMEM106B protein, human TMEM106B protein, mouse TMEM106B protein, and cynomolgus TMEM106B protein.

Human TMEM106B is a 274-amino acid protein that encodes a type 2 membrane glycoprotein. The amino acid sequence of human TMEM106B is set forth below (SEQ ID NO: 1):

MGKSLSHLPLHSSKEDAYDGVTSENMRNGLVNSEVHNEDGRNGDVS
QFPYVEFTGRDSVTCPTCQGTGRIPRGQENQLVALIPYSDQRLRP
RRTKLYVMASVFVCLLLSGLAVFFLFPRSIDVKYIGVKSAYVSYD
VQKRTIYLNITNTLNITNNNYYSVEVENITAQVQFSKTVIGKARL
NNITIIGPLDMKQIDYTVPTVIAEEMSYMYDFCTLISIKVHNIVL
MMQVTVTTTYFGHSEQISQERYQYVDCGRNTTYQLGQSEYLNVLQ
PQQ

In some embodiments, TMEM106B protein is expressed in a cell. In some embodiments, TMEM106B protein is expressed in endosomes and/or lysosomes. In some embodiments, TMEM106B protein is expressed in late endosomes and/or late lysosomes. In some embodiments, TMEM106B protein is expressed on the cell surface.

TMEM106B proteins of the present disclosure include several domains, including without limitation, an N-terminal luminal domain (predicted to range from amino acid residues 11-274 of human TMEM106B; see SEQ ID NO: 1), a transmembrane domain (predicted to range from amino acid residues 96-117 of human TMEM106B)), and a C-terminal domain (predicted to range from amino acid residues 1-92 of human TMEM106B).

### Epitope Binning

Epitope binning is a competitive immunoassay used to characterize and sort a library of monoclonal antibodies against a target protein (Abdiche et al, 2009, Analytical Biochemistry, 386:172-180). Epitope binning is also referred to as epitope mapping and epitope characterization (Brooks, 2014, Current Drug Discovery Technology, 11:109-112). Antibodies against a particular target (e.g., TMEM106B) are tested against all other antibodies in the library in a pairwise fashion to determine if any of the antibodies block one another's binding to an epitope of the target. After each antibody has a profile created against all of the other antibodies of the library, a competitive antibody blocking profile is created for each antibody relative to the other antibodies in the library. Closely related binning profiles indicate that the antibodies have the same or a closely related (e.g., overlapping) epitope and are “binned” together.

As shown in the Examples below, anti-TMEM106B antibodies of the present disclosure displayed a variety of binning profiles, characterized by bin 1, bin 5, and bin 2 (bin 2 includes related sub-bins 3 and 4).

Results provided herein show that anti-TMEM106B antibodies of the present disclosure that display overlapping or similar epitope binding characteristics (as evidenced by their binning profile) displayed varying degrees of effectiveness at reducing coronavirus cytopathic effect (CPE). In particular, anti-TMEM106B antibodies belonging to bin 2 (and related sub-bins 3 and 4) were effective at reducing cell death (as measured by CPE assay) following coronavirus infection in vitro compared to that observed with anti-TMEM106B antibodies belonging to bins 1 or 5.

### Coronavirus

Coronaviruses are a group of related enveloped positive-sense RNA viruses that cause disease in mammals and birds. Seven human coronaviruses (HCoVs) have been identified to date. These include four seasonally circulating human ‘common cold HCoVs’ and include the alphacoronaviruses 229E and NL63, and the betacoronaviruses OC43 and HKU1, each of which cause mild upper respiratory tract illnesses in humans. Three highly pathogenic coronaviruses emerged in the last two decades; these are the betacoronaviruses SARS-CoV, MERS-CoV, and the recently emerged SARS-CoV-2, each of which can cause severe, potentially lethal respiratory infections in humans. SARS-CoV-2 coronavirus is the virus responsible for Coronavirus Disease 2019 (COVID-19).

Upon receptor binding and membrane fusion to an infected cell, the coronavirus RNA is released into the cytoplasm, where it is translated to produce viral proteins. The viral replication/transcription complexes form on double-membrane vesicles within an infected cell and generate genome copies, which are then packaged into new virions via a budding process, through which they acquire the viral envelope, and the resulting virions are released from infected cells.

Coronaviruses mainly target epithelial cells (e.g., epithelial cells of the respiratory tract), and require certain host factors in order to infect a cell. Such host factors may play a role in one or more steps of the coronavirus replication cycle (e.g., receptor binding, endocytosis, fusion, translation of viral replication proteins and structural proteins, genome replication, virion assembly, and virion release). Infected individuals are able to shed virus into the environment, which can lead to virus transmission to other individuals. In humans, SARS-CoV-2 coronavirus infects epithelial cells of the respiratory tract via an aerosol route by binding to the angiotensin-converting enzyme 2 (ACE2) receptor.

TMEM106B has been identified as a host factor for SARS-CoV-2 coronavirus infection (Baggen et al, 2020, bioRxiv, doi:10.1101/2020.09.28.316281; Wang et al, 2020, bioRxiv, doi:10.1101/2020/09.24.312298; Wang et al, 2021, Cell, 184:1-14). Genetic deletion of TMEM106B and genome-wide loss-of-function screens in human cells identified host factors important for infection and replication of SARS-CoV-2 coronavirus. Specifically, deletion of TMEM106B reduced virus replication and reduced the cytopathic effect (CPE) of SARS-CoV-2 coronavirus in cultured human cell lines derived from liver and lung. CPE refers to structural changes in a host cell resulting from viral infection. CPE occurs when the infecting virus causes lysis (dissolution) of the host cell or when the host cell dies without lysis because of its inability to reproduce. If a virus causes these morphological changes in the host cell, it is said to be cytopathogenic. Common examples of CPE include rounding of the infected cell, fusion with adjacent cells to form syncytia, and the appearance of nuclear or cytoplasmic inclusion bodies.

Accordingly, in some embodiments, provided herein are methods and compositions for treating, preventing, or reducing the risk of coronavirus infection. In some embodiments, the methods and compositions provided herein are effective at treating, preventing, or reducing the risk of SARS-CoV-2 infection. In some embodiments that may be combined with any of the embodiments provided herein, the methods and compostions provided herein are effective at treating, preventing, or reducing the risk of SARS-CoV-2 infection, including variants of SARS-CoV-2 coronavirus, such as the alpha variant, the beta variant, the gamma variant, the delta variant, the lambda variant, etc.

In some embodiments that may be combined with any of the embodiments provided herein, an anti-TMEM106B antibody of the present disclosure reduces coronavirus replication. In some embodiments that may be combined with any of the embodiments provided herein, an anti-TMEM106B antibody of the present disclosure reduces coronavirus replication in vitro. In some embodiments that may be combined with any of the embodiments provided herein, an anti-TMEM106B antibody of the present disclosure reduces coronavirus replication in vivo. In some embodiments that may be combined with any of the embodiments provided herein, the coronavirus is SARS-CoV-2 coronavirus.

In other embodiments that may be combined with any of the embodiments provided herein, an anti-TMEM106B antibody of the present disclosure reduces coronavirus infection. In some embodiments that may be combined with any of the embodiments provided herein, an anti-TMEM106B antibody of the present disclosure reduces coronavirus infection in vitro. In some embodiments that may be combined with any of the embodiments provided herein, an anti-TMEM106B antibody of the present disclosure reduces coronavirus infection in vivo. In some embodiments that may be combined with any of the embodiments provided herein, the coronavirus is SARS-CoV-2 coronavirus.

In yet other embodiments that may be combined with any of the embodiments provided herein, an anti-TMEM106B antibody of the present disclosure reduces the cytopathic effect (CPE) of coronavirus. In some embodiments that may be combined with any of the embodiments provided herein, the coronavirus is SARS-CoV-2 coronavirus.

In some embodiments that may be combined with any of the embodiments provided herein, an anti-TMEM106B antibody of the present disclosure is effective at reducing coronavirus infection by at least 10%, by at least 15%, by at least 20%, by at least 25%, by at least 30%, by at least 35%, by at least 40%, by at least 45%, by at least 50%, by at least 55%, by at least 60%, by at least 65%, by at least 70%, by at least 75%, by at least 80%, by at least 85%, by at least 90%, or by at least 95%. In some embodiments that may be combined with any of the embodiments provided herein, the coronavirus is SARS-CoV-2 coronavirus.

In some embodiments that may be combined with any of the embodiments provided herein, an anti-TMEM106B antibody of the present disclosure is effective at reducing coronavirus replication by at least 10%, by at least 15%, by at least 20%, by at least 25%, by at least 30%, by at least 35%, by at least 40%, by at least 45%, by at least 50%, by at least 55%, by at least 60%, by at least 65%, by at least 70%, by at least 75%, by at least 80%, by at least 85%, by at least 90%, or by at least 95%. In some embodiments that may be combined with any of the embodiments provided herein, the coronavirus is SARS-CoV-2 coronavirus.

In some embodiments that may be combined with any of the embodiments provided herein, an anti-TMEM106B antibody of the present disclosure is effective at reducing coronavirus transmission by at least 10%, by at least 15%, by at least 20%, by at least 25%, by at least 30%, by at least 35%, by at least 40%, by at least 45%, by at least 50%, by at least 55%, by at least 60%, by at least 65%, by at least 70%, by at least 75%, by at least 80%, by at least 85%, by at least 90%, or by at least 95%. In some embodiments that may be combined with any of the embodiments provided herein, the coronavirus is SARS-CoV-2 coronavirus.

In some embodiments that may be combined with any of the embodiments provided herein, an anti-TMEM106B antibody of the present disclosure is effective at reducing coronavirus assembly by at least 10%, by at least 15%, by at least 20%, by at least 25%, by at least 30%, by at least 35%, by at least 40%, by at least 45%, by at least 50%, by at least 55%, by at least 60%, by at least 65%, by at least 70%, by at least 75%, by at least 80%, by at least 85%, by at least 90%, or by at least 95%. In some embodiments that may be combined with any of the embodiments provided herein, the coronavirus is SARS-CoV-2 coronavirus.

In some embodiments that may be combined with any of the embodiments provided herein, an anti-TMEM106B antibody of the present disclosure is effective at reducing coronavirus release from an infected cell by at least 10%, by at least 15%, by at least 20%, by at least 25%, by at least 30%, by at least 35%, by at least 40%, by at least 45%, by at least 50%, by at least 55%, by at least 60%, by at least 65%, by at least 70%, by at least 75%, by at least 80%, by at least 85%, by at least 90%, or by at least 95%. In some embodiments that may be combined with any of the embodiments provided herein, the coronavirus is SARS-CoV-2 coronavirus.

In some embodiments that may be combined with any of the embodiments provided herein, an anti-TMEM106B antibody of the present disclosure is effective at reducing coronavirus entry into a cell by at least 10%, by at least 15%, by at least 20%, by at least 25%, by at least 30%, by at least 35%, by at least 40%, by at least 45%, by at least 50%, by at least 55%, by at least 60%, by at least 65%, by at least 70%, by at least 75%, by at least 80%, by at least 85%, by at least 90%, or by at least 95%. In some embodiments that may be combined with any of the embodiments provided herein, the coronavirus is SARS-CoV-2 coronavirus.

In some embodiments that may be combined with any of the embodiments provided herein, an anti-TMEM106B antibody of the present disclosure is effective at reducing coronavirus genome translation by at least 10%, by at least 15%, by at least 20%, by at least 25%, by at least 30%, by at least 35%, by at least 40%, by at least 45%, by at least 50%, by at least 55%, by at least 60%, by at least 65%, by at least 70%, by at least 75%, by at least 80%, by at least 85%, by at least 90%, or by at least 95%. In some embodiments that may be combined with any of the embodiments provided herein, the coronavirus is SARS-CoV-2 coronavirus.

### Exemplary Anti-TMEM106B Antibodies and Certain Other Antibody Embodiments

The present disclosure provides anti-TMEM106B antibodies for use in treating, preventing, or reducing the risk of coronavirus infection, including anti-TMEM106B antibodies TM-1, TM-2, TM-3, TM-4, TM-5, TM-6, TM-7, TM-8, TM-9, TM-10, TM-11, TM-12, TM-13, TM-14, TM-15, TM-16, TM-17, TM-18, TM-19, TM-20, TM-21, TM-22, TM-23, TM-24, TM-25, TM-26, TM-27, TM-28, TM-29, TM-30, TM-31, TM-32, TM-33, TM-34, TM-35, TM-37, TM-39, TM-41, TM-42, TM-43, TM-44, TM-45, TM-46, TM-47, TM-48, TM-49, TM-50, TM-51, TM-52, TM-54, TM-56, TM-59, TM-60, TM-61, TM-62, TM-63, TM-64, TM-65, TM-66, TM-67, TM-68, TM-69, TM-70, TM-71, TM-72, TM-73, TM-74, TM-75, TM-76, TM-77, TM-78, TM-79, TM-80. TM-81, TM-82, TM-83, TM-84, TM-85, TM-86, TM-87, TM-88, TM-89, TM90, TM-91, TM-92, TM-93, and TM-94. The amino acid sequences of exemplary anti-TMEM106B antibodies are provided below in Tables 1-6.

In some aspects, an anti-TMEM106B antibody useful in the methods of the present disclosure comprises a heavy chain variable domain (V_{H}) amino acid sequence having at least 90%, 91%, 92%, 93%, 94%, 95%, 96%, 97%, 98%, 99%, or 100% amino acid sequence identity to the amino acid sequence of the heavy chain variable domain (V_{H}) of an anti-TMEM106B antibody selected from the group consisting of TM-1, TM-2, TM-3, TM-4, TM-5, TM-6, TM-7, TM-8, TM-9, TM-10, TM-11, TM-12, TM-13, TM-14, TM-15, TM-16, TM-17, TM-18, TM-19, TM-20, TM-21, TM-22, TM-23, TM-24, TM-25, TM-26, TM-27, TM-28, TM-29, TM-30, TM-31, TM-32, TM-33, TM-34, TM-35, TM-37, TM-39, TM-41, TM-42, TM-43, TM-44, TM-45, TM-46, TM-47, TM-48, TM-49, TM-50, TM-51, TM-52, TM-54, TM-56, TM-59, TM-60, TM-61, TM-62, TM-63, TM-64, TM-65, TM-66, TM-67, TM-68, TM-69, TM-70, TM-71, TM-72, TM-73, TM-74, TM-75, TM-76, TM-77, TM-78, TM-79, TM-80. TM-81, TM-82, TM-83, TM-84, TM-85, TM-86, TM-87, TM-88, TM-89, TM90, TM-91, TM-92, TM-93, and TM-94. In certain embodiments, a V_{H} amino acid sequence having at least 90%, 91%, 92%, 93%, 94%, 95%, 96%, 97%, 98%, or 99% identity to the amino acid sequence of the heavy chain variable domain (V_{H}) of an anti-TMEM106B antibody selected from the group consisting of TM-1, TM-2, TM-3, TM-4, TM-5, TM-6, TM-7, TM-8, TM-9, TM-10, TM-11, TM-12, TM-13, TM-14, TM-15, TM-16, TM-17, TM-18, TM-19, TM-20, TM-21, TM-22, TM-23, TM-24, TM-25, TM-26, TM-27, TM-28, TM-29, TM-30, TM-31, TM-32, TM-33, TM-34, TM-35, TM-37, TM-39, TM-41, TM-42, TM-43, TM-44, TM-45, TM-46, TM-47, TM-48, TM-49, TM-50, TM-51, TM-52, TM-54, TM-56, TM-59, TM-60, TM-61, TM-62, TM-63, TM-64, TM-65, TM-66, TM-67, TM-68, TM-69, TM-70, TM-71, TM-72, TM-73, TM-74, TM-75, TM-76, TM-77, TM-78, TM-79, TM-80. TM-81, TM-82, TM-83, TM-84, TM-85, TM-86, TM-87, TM-88, TM-89, TM90, TM-91, TM-92, TM-93, and TM-94 contains substitutions (e.g., conservative substitutions), insertions, or deletions relative to the reference sequence, but an anti-TMEM106B antibody comprising that sequence retains the ability to bind to TMEM106B. In certain embodiments, a total of 1 to 10 amino acids have been substituted, inserted, and/or deleted in the amino acid sequence of the heavy chain variable domain (V_{H}) of an anti-TMEM106B antibody selected from the group consisting of TM-1, TM-2, TM-3, TM-4, TM-5, TM-6, TM-7, TM-8, TM-9, TM-10, TM-11, TM-12, TM-13, TM-14, TM-15, TM-16, TM-17, TM-18, TM-19, TM-20, TM-21, TM-22, TM-23, TM-24, TM-25, TM-26, TM-27, TM-28, TM-29, TM-30, TM-31, TM-32, TM-33, TM-34, TM-35, TM-37, TM-39, TM-41, TM-42, TM-43, TM-44, TM-45, TM-46, TM-47, TM-48, TM-49, TM-50, TM-51, TM-52, TM-54, TM-56, TM-59, TM-60, TM-61, TM-62, TM-63, TM-64, TM-65, TM-66, TM-67, TM-68, TM-69, TM-70, TM-71, TM-72, TM-73, TM-74, TM-75, TM-76, TM-77, TM-78, TM-79, TM-80. TM-81, TM-82, TM-83, TM-84, TM-85, TM-86, TM-87, TM-88, TM-89, TM90, TM-91, TM-92, TM-93, and TM-94. In certain embodiments, a total of 1 to 5 amino acids have been substituted, inserted and/or deleted in the amino acid sequence of the heavy chain variable domain (V_{H}) of an anti-TMEM106B antibody selected form the group consisting of TM-1, TM-2, TM-3, TM-4, TM-5, TM-6, TM-7, TM-8, TM-9, TM-10, TM-11, TM-12, TM-13, TM-14, TM-15, TM-16, TM-17, TM-18, TM-19, TM-20, TM-21, TM-22, TM-23, TM-24, TM-25, TM-26, TM-27, TM-28, TM-29, TM-30, TM-31, TM-32, TM-33, TM-34, TM-35, TM-37, TM-39, TM-41, TM-42, TM-43, TM-44, TM-45, TM-46, TM-47, TM-48, TM-49, TM-50, TM-51, TM-52, TM-54, TM-56, TM-59, TM-60, TM-61, TM-62, TM-63, TM-64, TM-65, TM-66, TM-67, TM-68, TM-69, TM-70, TM-71, TM-72, TM-73, TM-74, TM-75, TM-76, TM-77, TM-78, TM-79, TM-80. TM-81, TM-82, TM-83, TM-84, TM-85, TM-86, TM-87, TM-88, TM-89, TM90, TM-91, TM-92, TM-93, and TM-94. In certain embodiments, substitutions, insertions, or deletions occur in regions outside the HVRs (i.e., in the FRs). Optionally, an anti-TMEM106B antibody of the present disclosure comprises the V_{H} amino acid sequence of the heavy chain variable domain (V_{H}) of an anti-TMEM106B antibody selected from the group consisting of TM-1, TM-2, TM-3, TM-4, TM-5, TM-6, TM-7, TM-8, TM-9, TM-10, TM-11, TM-12, TM-13, TM-14, TM-15, TM-16, TM-17, TM-18, TM-19, TM-20, TM-21, TM-22, TM-23, TM-24, TM-25, TM-26, TM-27, TM-28, TM-29, TM-30, TM-31, TM-32, TM-33, TM-34, TM-35, TM-37, TM-39, TM-41, TM-42, TM-43, TM-44, TM-45, TM-46, TM-47, TM-48, TM-49, TM-50, TM-51, TM-52, TM-54, TM-56, TM-59, TM-60, TM-61, TM-62, TM-63, TM-64, TM-65, TM-66, TM-67, TM-68, TM-69, TM-70, TM-71, TM-72, TM-73, TM-74, TM-75, TM-76, TM-77, TM-78, TM-79, TM-80. TM-81, TM-82, TM-83, TM-84, TM-85, TM-86, TM-87, TM-88, TM-89, TM90, TM-91, TM-92, TM-93, and TM-94, including post-translational modifications of that sequence. In certain embodiments, the V_{H} comprises an amino acid sequence provided in Table 5 and Table 6 below. In certain embodiments, the V_{H} comprises one, two or three HVRs selected from the HVR-H1, HVR-H2, and HVR-H3 amino acid sequences provided in Table 1 and Table 3 below.

In another aspect, an anti-TMEM106B antibody useful in the methods of the present disclosure comprises a light chain variable domain (V_{L}) amino acid sequence having at least 90%, 91%, 92%, 93%, 94%, 95%, 96%, 97%, 98%, 99%, or 100% sequence identity to the amino acid sequence of the light chain variable domain (V_{L}) of an anti-TMEM106B antibody selected from the group consisting of anti-TMEM106B antibody TM-1, TM-2, TM-3, TM-4, TM-5, TM-6, TM-7, TM-8, TM-9, TM-10, TM-11, TM-12, TM-13, TM-14, TM-15, TM-16, TM-17, TM-18, TM-19, TM-20, TM-21, TM-22, TM-23, TM-24, TM-25, TM-26, TM-27, TM-28, TM-29, TM-30, TM-31, TM-32, TM-33, TM-34, TM-35, TM-37, TM-39, TM-41, TM-42, TM-43, TM-44, TM-45, TM-46, TM-47, TM-48, TM-49, TM-50, TM-51, TM-52, TM-54, TM-56, TM-59, TM-60, TM-61, TM-62, TM-63, TM-64, TM-65, TM-66, TM-67, TM-68, TM-69, TM-70, TM-71, TM-72, TM-73, TM-74, TM-75, TM-76, TM-77, TM-78, TM-79, TM-80. TM-81, TM-82, TM-83, TM-84, TM-85, TM-86, TM-87, TM-88, TM-89, TM90, TM-91, TM-92, TM-93, and TM-94. In certain embodiments, a V_{L} amino acid sequence having at least 90%, 91%, 92%, 93%, 94%, 95%, 96%, 97%, 98%, or 99% identity to the amino acid sequence of the light chain variable domain (V_{L}) of an anti-TMEM106B antibody selected from the group consisting of anti-TMEM106B antibody TM-1, TM-2, TM-3, TM-4, TM-5, TM-6, TM-7, TM-8, TM-9, TM-10, TM-11, TM-12, TM-13, TM-14, TM-15, TM-16, TM-17, TM-18, TM-19, TM-20, TM-21, TM-22, TM-23, TM-24, TM-25, TM-26, TM-27, TM-28, TM-29, TM-30, TM-31, TM-32, TM-33, TM-34, TM-35, TM-37, TM-39, TM-41, TM-42, TM-43, TM-44, TM-45, TM-46, TM-47, TM-48, TM-49, TM-50, TM-51, TM-52, TM-54, TM-56, TM-59, TM-60, TM-61, TM-62, TM-63, TM-64, TM-65, TM-66, TM-67, TM-68, TM-69, TM-70, TM-71, TM-72, TM-73, TM-74, TM-75, TM-76, TM-77, TM-78, TM-79, TM-80. TM-81, TM-82, TM-83, TM-84, TM-85, TM-86, TM-87, TM-88, TM-89, TM90, TM-91, TM-92, TM-93, and TM-94 contains substitutions (e.g., conservative substitutions), insertions, or deletions relative to the reference sequence, but an anti-TMEM106B antibody comprising that sequence retains the ability to bind to TMEM106B. In some embodiments, a total of 1 to 10 amino acids have been substituted, inserted and/or deleted in the amino acid sequence of the light chain variable domain (V_{L}) of an anti-TMEM106B antibody selected from the group consisting of TM-1, TM-2, TM-3, TM-4, TM-5, TM-6, TM-7, TM-8, TM-9, TM-10, TM-11, TM-12, TM-13, TM-14, TM-15, TM-16, TM-17, TM-18, TM-19, TM-20, TM-21, TM-22, TM-23, TM-24, TM-25, TM-26, TM-27, TM-28, TM-29, TM-30, TM-31, TM-32, TM-33, TM-34, TM-35, TM-37, TM-39, TM-41, TM-42, TM-43, TM-44, TM-45, TM-46, TM-47, TM-48, TM-49, TM-50, TM-51, TM-52, TM-54, TM-56, TM-59, TM-60, TM-61, TM-62, TM-63, TM-64, TM-65, TM-66, TM-67, TM-68, TM-69, TM-70, TM-71, TM-72, TM-73, TM-74, TM-75, TM-76, TM-77, TM-78, TM-79, TM-80. TM-81, TM-82, TM-83, TM-84, TM-85, TM-86, TM-87, TM-88, TM-89, TM90, TM-91, TM-92, TM-93, and TM-94. In certain embodiments, a total of 1 to 5 amino acids have been substituted, inserted and/or deleted in the amino acid sequence of the light chain variable domain (V_{L}) of an anti-TMEM106B antibody selected from the group consisting of TM-1, TM-2, TM-3, TM-4, TM-5, TM-6, TM-7, TM-8, TM-9, TM-10, TM-11, TM-12, TM-13, TM-14, TM-15, TM-16, TM-17, TM-18, TM-19, TM-20, TM-21, TM-22, TM-23, TM-24, TM-25, TM-26, TM-27, TM-28, TM-29, TM-30, TM-31, TM-32, TM-33, TM-34, TM-35, TM-37, TM-39, TM-41, TM-42, TM-43, TM-44, TM-45, TM-46, TM-47, TM-48, TM-49, TM-50, TM-51, TM-52, TM-54, TM-56, TM-59, TM-60, TM-61, TM-62, TM-63, TM-64, TM-65, TM-66, TM-67, TM-68, TM-69, TM-70, TM-71, TM-72, TM-73, TM-74, TM-75, TM-76, TM-77, TM-78, TM-79, TM-80. TM-81, TM-82, TM-83, TM-84, TM-85, TM-86, TM-87, TM-88, TM-89, TM90, TM-91, TM-92, TM-93, and TM-94. In certain embodiments, the substitutions, insertions, or deletions occur in regions outside the HVRs (i.e., in the FRs). Optionally, an anti-TMEM106B antibody of the present disclosure comprises the V_{L} sequence of the light chain variable domain (V_{L}) of an anti-TMEM106B antibody selected from the group consisting of TM-1, TM-2, TM-3, TM-4, TM-5, TM-6, TM-7, TM-8, TM-9, TM-10, TM-11, TM-12, TM-13, TM-14, TM-15, TM-16, TM-17, TM-18, TM-19, TM-20, TM-21, TM-22, TM-23, TM-24, TM-25, TM-26, TM-27, TM-28, TM-29, TM-30, TM-31, TM-32, TM-33, TM-34, TM-35, TM-37, TM-39, TM-41, TM-42, TM-43, TM-44, TM-45, TM-46, TM-47, TM-48, TM-49, TM-50, TM-51, TM-52, TM-54, TM-56, TM-59, TM-60, TM-61, TM-62, TM-63, TM-64, TM-65, TM-66, TM-67, TM-68, TM-69, TM-70, TM-71, TM-72, TM-73, TM-74, TM-75, TM-76, TM-77, TM-78, TM-79, TM-80. TM-81, TM-82, TM-83, TM-84, TM-85, TM-86, TM-87, TM-88, TM-89, TM90, TM-91, TM-92, TM-93, and TM-94, including post-translational modifications of that sequence. In certain embodiments, the V_{L} comprises an amino acid sequence provided in Table 5 and Table 6 below. In certain embodiments, the V_{L} comprises one, two or three HVRs selected from the HVR-L1, HVR-L2, and HVR-L3 amino acid sequences provided in Table 2 and Table 4 below.

In some embodiments, an anti-TMEM106B antibody is provided for use in the methods described herein, wherein the antibody comprises a V H as in any of the embodiments provided above, and a V_{L} as in any of the embodiments provided above. In some embodiments, provided herein are anti-TMEM106B antibodies, wherein the antibody comprises a V H as in any of the embodiments provided above, and a V_{L} as in any of the embodiments provided above. In one embodiment, the antibody comprises the V_{H} and V_{L} sequences of anti-TMEM106B antibody selected from the group consisting of anti-TMEM106B antibodies TM-1, TM-2, TM-3, TM-4, TM-5, TM-6, TM-7, TM-8, TM-9, TM-10, TM-11, TM-12, TM-13, TM-14, TM-15, TM-16, TM-17, TM-18, TM-19, TM-20, TM-21, TM-22, TM-23, TM-24, TM-25, TM-26, TM-27, TM-28, TM-29, TM-30, TM-31, TM-32, TM-33, TM-34, TM-35, TM-37, TM-39, TM-41, TM-42, TM-43, TM-44, TM-45, TM-46, TM-47, TM-48, TM-49, TM-50, TM-51, TM-52, TM-54, TM-56, TM-59, TM-60, TM-61, TM-62, TM-63, TM-64, TM-65, TM-66, TM-67, TM-68, TM-69, TM-70, TM-71, TM-72, TM-73, TM-74, TM-75, TM-76, TM-77, TM-78, TM-79, TM-80. TM-81, TM-82, TM-83, TM-84, TM-85, TM-86, TM-87, TM-88, TM-89, TM90, TM-91, TM-92, TM-93, and TM-94, including post-translational modifications of that sequence.

In some embodiments, an antibody competitively inhibits the binding to TMEM106B of an antibody comprising the heavy chain variable region and the light chain variable region of any one of the anti-TMEM106B antibodies selected from the group consisting of TM-1, TM-2, TM-3, TM-4, TM-5, TM-6, TM-7, TM-8, TM-9, TM-10, TM-11, TM-12, TM-13, TM-14, TM-15, TM-16, TM-17, TM-18, TM-19, TM-20, TM-21, TM-22, TM-23, TM-24, TM-25, TM-26, TM-27, TM-28, TM-29, TM-30, TM-31, TM-32, TM-33, TM-34, TM-35, TM-37, TM-39, TM-41, TM-42, TM-43, TM-44, TM-45, TM-46, TM-47, TM-48, TM-49, TM-50, TM-51, TM-52, TM-54, TM-56, TM-59, TM-60, TM-61, TM-62, TM-63, TM-64, TM-65, TM-66, TM-67, TM-68, TM-69, TM-70, TM-71, TM-72, TM-73, TM-74, TM-75, TM-76, TM-77, TM-78, TM-79, TM-80. TM-81, TM-82, TM-83, TM-84, TM-85, TM-86, TM-87, TM-88, TM-89, TM90, TM-91, TM-92, TM-93, and TM-94, including post-translational modifications of that sequence. In some embodiments, the antibody binds a truncated TMEM106B protein comprising amino acids 122-210 of SEQ ID NO:1. In some embodiments, the antibody is in Bin 2. In some embodiments, the antibody is in Bin 3. In some embodiments, the antibody is in Bin 4. In some embodiments, the antibody is not in Bin 1. In some embodiments, the antibody is not in Bin 5.

Anti-TMEM106B antibodies for use in the methods of the present disclosure may bind to various regions of TMEM106B, including various regions of human TMEM106B. Such regions of TMEM106B include the cytoplasmic domain of TMEM106B or the luminal domain TMEM106B.

In some embodiments, an anti-TMEM106B antibody for use in the methods of the present disclosure binds to one or more regions or domains of TMEM106B. In some embodiments, an anti-TMEM106B antibody for use in the methods of the present disclosure binds to one or more regions or domains of human TMEM106B.

In some embodiments, an anti-TMEM106B antibody according to any of the above embodiments is a monoclonal antibody, including a humanized and/or human antibody. In some embodiments, the anti-TMEM106B antibody is an antibody fragment, e.g., a Fv, Fab, Fab′, scFv, diabody, or F(ab′)2 fragment. In some embodiments, the anti-TMEM106B antibody is a substantially full-length antibody, e.g., an IgG1 antibody, IgG2a antibody or other antibody class or isotype as defined herein.

In some embodiments, an anti-TMEM106B antibody useful in the any of the methods according to any of the above embodiments may incorporate any of the features, singly or in combination, as described in Sections 1-7 below:

### (1) Anti-TMEM106B Antibody Binding Affinity

In some embodiments of any of the antibodies provided herein, the antibody has a dissociation constant (Kd) of <1 μM, <100 nM, <10 nM, <1 nM, <0.1 nM, <0.01 nM, or <0.001 nM (e.g., 10⁻⁸ M or less, e.g., from 10⁻⁸ M to 10⁻¹³ M, e.g., from 10⁻⁹ M to 10⁻¹³ M). Dissociation constants may be determined through any analytical technique, including any biochemical or biophysical technique such as ELISA, surface plasmon resonance (SPR), bio-layer interferometry (see, e.g., Octet System by ForteBio), isothermal titration calorimetry (ITC), differential scanning calorimetry (DSC), circular dichroism (CD), stopped-flow analysis, and colorimetric or fluorescent protein melting analyses. In one embodiment, Kd is measured by a radiolabeled antigen binding assay (RIA). In some embodiment, an RIA is performed with the Fab version of an antibody of interest and its antigen, for example as described in Chen et al. J. Mol. Biol. 293:865-881(1999)). In some embodiments, Kd is measured using a BIACORE surface plasmon resonance assay, for example, an assay using a BIACORE -2000 or a BIACORE -3000 (BlAcore, Inc., Piscataway, NJ) is performed at 25° C. with immobilized antigen CM5 chips at ˜10 response units (RU). In some embodiments, the K_{D} is determined using a monovalent antibody (e.g., a Fab) or a full-length antibody. In some embodiments, the K_{D} is determined using a full-length antibody in a monovalent form.

In some embodiments, an anti-TMEM106B antibody of the present disclosure may have nanomolar or even picomolar affinities for TMEM106B. In some embodiments, the dissociation constant (Kd) of the antibody is about 0.1 nM to about 500 nM. For example, the Kd of the antibody is any of about 500 nM, about 400 nM, about 300 nM, about 200 nM, about 100 nM, about 75 nM, about 50 nM, about 25 nM, about 10 nM, about 9 nM, about 8 nM, about 7 nM, about 6 nM, about 5 nM, about 4 nM, about 3 nM, about 2 nM, about 1 nM, or about 1 nM to about 0.1 nM for binding to human TMEM106B.

### (2) Antibody Fragments

In some embodiments of any of the antibodies provided herein, the antibody is an antibody fragment. Antibody fragments include, but are not limited to, Fab, Fab′, Fab′-SH, F(ab′)₂, Fv, and scFv fragments, and other fragments described below. For a review of certain antibody fragments, see Hudson et al. Nat. Med. 9:129-134 (2003). For a review of scFv fragments, see, e.g., WO 93/16185; and U.S. Pat. Nos. 5,571,894 and 5,587,458. For discussion of Fab and F(ab′)₂ fragments comprising salvage receptor binding epitope residues and having increased in vivo half-life, see U.S. Pat. No. 5,869,046.

Diabodies are antibody fragments with two antigen-binding sites that may be bivalent or bispecific. See, for example, EP404097; WO 1993/01161; Hudson et al. Nat. Med. 9:129-134 (2003). Triabodies and tetrabodies are also described in Hudson et al. Nat. Med. 9:129-134 (2003). Single-domain antibodies are antibody fragments comprising all or a portion of the heavy chain variable domain or all or a portion of the light chain variable domain of an antibody. In certain embodiments, a single-domain antibody is a human single-domain antibody (see, e.g., U.S. Pat. No. 6,248,516).

Antibody fragments can be made by various techniques, including but not limited to proteolytic digestion of an intact antibody as well as production by recombinant host cells (e.g., E. coli or phage), as described herein.

### (3) Chimeric and Humanized Antibodies

In some embodiments of any of the antibodies provided herein, the antibody is a chimeric antibody. Certain chimeric antibodies are described, e.g., in U.S. Pat. No. 4,816,567. In one example, a chimeric antibody comprises a non-human variable region (e.g., a variable region derived from a mouse, rat, hamster, rabbit, or non-human primate, such as a monkey) and a human constant region. In a further example, a chimeric antibody is a “class switched” antibody in which the class or subclass has been changed from that of the parent antibody. Chimeric antibodies include antigen-binding fragments thereof.

In some embodiments of any of the antibodies provided herein, the antibody is a humanized antibody. Typically, a non-human antibody is humanized to reduce immunogenicity to humans, while retaining the specificity and affinity of the parental non-human antibody. In certain embodiments, a humanized antibody is substantially non-immunogenic in humans. In certain embodiments, a humanized antibody has substantially the same affinity for a target as an antibody from another species from which the humanized antibody is derived. See, e.g., U.S. Pat. Nos. 5,530,101, 5,693,761; 5,693,762; and 5,585,089. In certain embodiments, amino acids of an antibody variable domain that can be modified without diminishing the native affinity of the antigen binding domain while reducing its immunogenicity are identified. See, e.g., U.S. Pat. Nos. 5,766,886 and 5,869,619. Generally, a humanized antibody comprises one or more variable domains in which HVRs (or portions thereof) are derived from a non-human antibody, and FRs (or portions thereof) are derived from human antibody sequences. A humanized antibody optionally will also comprise at least a portion of a human constant region. In some embodiments, some FR residues in a humanized antibody are substituted with corresponding residues from a non-human antibody (e.g., the antibody from which the HVR residues are derived), for example, to restore or improve antibody specificity or affinity.

Humanized antibodies and methods of making them are reviewed, for example, in Almagro et al. Front. Biosci. 13:161 9-1633 (2008), and are further described, e.g., in U.S. Pat. Nos. 5,821,337, 7,527,791, 6,982,321, and 7,087,409. Human framework regions that may be used for humanization include but are not limited to: framework regions selected using the “best-fit” method (see, e.g., Sims et al. J. Immunol. 151:2296 (1993)); framework regions derived from the consensus sequence of human antibodies of a particular subgroup of light or heavy chain variable regions (see, e.g., Carter et al. Proc. Natl. Acad. Sci. USA 89:4285 (1992); and Presta et al., J. Immunol. 151 :2623 (1993)); human mature (somatically mutated) framework regions or human germline framework regions (see, e.g., Almagro and Fransson Front. Biosci. 13:1619-1633 (2008)); and framework regions derived from screening FR libraries (see, e.g., Baca et al. J. Biol. Chem. 272:10678-10684 (1997) and Rosok et al. J. Biol. Chem. 271:22611-22618 (1996)).

### (4) Human Antibodies

In some embodiments of any of the antibodies provided herein, the antibody is a human antibody. Human antibodies can be produced using various techniques known in the art. Human antibodies are described generally in van Dijk et al. Curr. Opin. Pharmacol. 5:368-74 (2001) and Lonberg Curr. Opin. Immunol. 20:450-459 (2008).

Human antibodies may be prepared by administering an immunogen to a transgenic animal that has been modified to produce intact human antibodies or intact antibodies with human variable regions in response to antigenic challenge. One can engineer mouse strains deficient in mouse antibody production with large fragments of the human Ig loci in anticipation that such mice would produce human antibodies in the absence of mouse antibodies. Large human Ig fragments can preserve the large variable gene diversity as well as the proper regulation of antibody production and expression. By exploiting the mouse machinery for antibody diversification and selection and the lack of immunological tolerance to human proteins, the reproduced human antibody repertoire in these mouse strains can yield high affinity fully human antibodies against any antigen of interest, including human antigens. Using the hybridoma technology, antigen-specific human monoclonal antibodies with the desired specificity can be produced and selected. Certain exemplary methods are described in U.S. Pat. No. 5,545,807, EP 546073, and EP 546073. See also, for example, U.S. Pat. Nos. 6,075,181 and 6,150,584 describing XENOMOUSE™ technology; U.S. Pat. No. 5,770,429 describing HUMAB® technology; U.S. Pat. No. 7,041,870 describing K-M MOUSE® technology, and U.S. Patent Application Publication No. US 2007/0061900, describing VELOCIMOUSE® technology. Human variable regions from intact antibodies generated by such animals may be further modified, e.g., by combining with a different human constant region.

Human antibodies can also be made by hybridoma-based methods. Human myeloma and mouse-human heteromyeloma cell lines for the production of human monoclonal antibodies have been described. (See, e.g., Kozbor J. Immunol. 133:3001 (1984) and Boerner et al. J. Immunol. 147:86 (1991)). Human antibodies generated via human B-cell hybridoma technology are also described in Li et al.Proc. Natl. Acad. Sci. USA, 1 03:3557-3562 (2006). Additional methods include those described, for example, in U.S. Pat. No. 7,189,826 (describing production of monoclonal human IgM antibodies from hybridoma cell lines). Human hybridoma technology (Trioma technology) is also described in Vollmers et al. Histology and Histopathology 20(3) :927-937 (2005) and Vollmers et al. Methods and Findings in Experimental and Clinical Pharmacology 27(3):185-91 (2005). Human antibodies may also be generated by isolating Fv clone variable domain sequences selected from human-derived phage display libraries. Such variable domain sequences may then be combined with a desired human constant domain. Techniques for selecting human antibodies from antibody libraries are described below.

In some embodiments of any of the antibodies provided herein, the antibody is a human antibody isolated by in vitro methods and/or screening combinatorial libraries for antibodies with the desired activity or activities. Suitable examples include but are not limited to phage display (CAT, Morphosys, Dyax, Biosite/Medarex, Xoma, Symphogen, Alexion (formerly Proliferon), Affimed) ribosome display (CAT), yeast display (Adimab), and the like. In certain phage display methods, repertoires of V_{H} and V_{L} genes are separately cloned by polymerase chain reaction (PCR) and recombined randomly in phage libraries, which can then be screened for antigen-binding phage as described in Winter et al. Ann. Rev. Immunol. 12: 433-455 (1994). For example, a variety of methods are known in the art for generating phage display libraries and screening such libraries for antibodies possessing the desired binding characteristics. See also Sidhu et al. J Mol. Biol. 338(2): 299-310, 2004; Lee et al. J Mol. Biol. 340(5): 1073-1093, 2004; Fellouse Proc. Natl. Acad. Sci. USA 101(34):12467-12472 (2004); and Lee et al. J Immunol. Methods 284(-2):1 19-132 (2004). Phage typically display antibody fragments, either as single-chain Fv (scFv) fragments or as Fab fragments. Libraries from immunized sources provide high-affinity antibodies to the immunogen without the requirement of constructing hybridomas. Alternatively, the naive repertoire can be cloned (e.g., from human) to provide a single source of antibodies to a wide range of non-self and also self-antigens without any immunization as described by Griffiths et al. EMBO J. 12: 725-734 (1993). Finally, naive libraries can also be made synthetically by cloning unrearranged V-gene segments from stem cells, and using PCR primers comprising random sequence to encode the highly variable HVR3 regions and to accomplish rearrangement in vitro, as described by Hoogenboom et al. J. Mol. Biol., 227: 381-388, 1992. Patent publications describing human antibody phage libraries include, for example: U.S. Pat. No. 5,750,373, and US Patent Publication Nos. 2007/0292936 and 2009/0002360. Antibodies isolated from human antibody libraries are considered human antibodies or human antibody fragments herein.

### (5) Constant Regions Including Fc Regions

In some embodiments of any of the antibodies provided herein, the antibody comprises an Fc. In some embodiments, the Fc is a human IgG1, IgG2, IgG3, and/or IgG4 isotype. In some embodiments, the antibody is of the IgG class, the IgM class, or the IgA class.

In certain embodiments of any of the antibodies provided herein, the antibody has an IgG2 isotype. In some embodiments, the antibody contains a human IgG2 constant region. In some embodiments, the human IgG2 constant region includes an Fc region. In some embodiments, the antibody induces the one or more TMEM106B activities or independently of binding to an Fc receptor. In some embodiments, the antibody binds an inhibitory Fc receptor. In certain embodiments, the inhibitory Fc receptor is inhibitory Fc-gamma receptor IIB (FcγIIB).

In certain embodiments of any of the antibodies provided herein, the antibody has an IgG1 isotype. In some embodiments, the antibody contains a mouse IgG1 constant region. In some embodiments, the antibody contains a human IgG1 constant region. In some embodiments, the human IgG1 constant region includes an Fc region. In some embodiments, the antibody binds an inhibitory Fc receptor. In certain embodiments, the inhibitory Fc receptor is inhibitory Fc-gamma receptor JIB (FcγIIB).

In certain embodiments of any of the antibodies provided herein, the antibody has an IgG4 isotype. In some embodiments, the antibody contains a human IgG4 constant region. In some embodiments, the human IgG4 constant region includes an Fc region. In some embodiments, the antibody binds an inhibitory Fc receptor. In certain embodiments, the inhibitory Fc receptor is inhibitory Fc-gamma receptor IIB (FcγIIB).

In certain embodiments of any of the antibodies provided herein, the antibody has a hybrid IgG2/4 isotype. In some embodiments, the antibody includes an amino acid sequence comprising amino acids 118 to 260 according to EU numbering of human IgG2 and amino acids 261-447 according to EU numbering of human IgG4 (WO 1997/11971; WO 2007/106585).

In some embodiments, the Fc region increases clustering without activating complement as compared to a corresponding antibody comprising an Fc region that does not comprise the amino acid substitutions. In some embodiments, the antibody induces one or more activities of a target specifically bound by the antibody. In some embodiments, the antibody binds to TMEM106B.

It may also be desirable to modify an anti-TMEM106B antibody of the present disclosure to modify effector function and/or to increase serum half-life of the antibody. For example, the Fc receptor binding site on the constant region may be modified or mutated to remove or reduce binding affinity to certain Fc receptors, such as FcγRI, FcγRII, and/or FcγRIII to reduce Antibody-dependent cell-mediated cytotoxicity. In some embodiments, the effector function is impaired by removing N-glycosylation of the Fc region (e.g., in the CH2 domain of IgG) of the antibody. In some embodiments, the effector function is impaired by modifying regions such as 233-236, 297, and/or 327-331 of human IgG as described in WO 99/58572 and Armour et al. Molecular Immunology 40: 585-593 (2003); Reddy et al. J. Immunology 164:1925-1933 (2000). In other embodiments, it may also be desirable to modify an anti-TMEM106B antibody of the present disclosure to modify effector function to increase finding selectivity toward the ITIM-containing FcgRilb (CD32b) to increase clustering of TMEM106B antibodies on adjacent cells without activating humoral responses including Antibody-dependent cell-mediated cytotoxicity and antibody-dependent cellular phagocytosis.

To increase the serum half-life of the antibody, one may incorporate a salvage receptor binding epitope into the antibody (especially an antibody fragment) as described in U.S. Pat. No. 5,739,277, for example. As used herein, the term “salvage receptor binding epitope” refers to an epitope of the Fc region of an IgG molecule (e.g., IgG₁, IgG₂, IgG₃, or IgG₄) that is responsible for increasing the in vivo serum half-life of the IgG molecule. Other amino acid sequence modifications.

### (6) Multispecific Antibodies

Multispecific antibodies are antibodies that have binding specificities for at least two different epitopes, including those on the same or another polypeptide (e.g., one or more TMEM106B polypeptides of the present disclosure). In some embodiments, the multispecific antibody can be a bispecific antibody. In some embodiments, the multispecific antibody can be a trispecific antibody. In some embodiments, the multispecific antibody can be a tetraspecific antibody. Such antibodies can be derived from full-length antibodies or antibody fragments (e.g., F(ab′)₂ bispecific antibodies). In some embodiments, the multispecific antibody comprises a first antigen binding region which binds to first site on TMEM106B and comprises a second antigen binding region which binds to a second site on TMEM106B. In some embodiment, the multispecific antibodies comprises a first antigen binding region which binds to TMEM106B and a second antigen binding region that binds to a second polypeptide.

Provided herein are multispecific antibodies comprises a first antigen binding region, wherein the first antigen binding region comprises the six HVRs of an antibody described herein, which binds to TMEM106B and a second antigen binding region that binds to a second polypeptide. In some embodiments, the first antigen binding region comprises the V_{H} or V_{L} of an antibody described herein.

The multivalent antibodies may recognize the TMEM106B antigen as well as without limitation additional antigens, such as a coronavirus viral entry factor, including angiotensin-converting enzyme 2 (ACE2), which is a viral entry receptor for HCoV-NL63, SARS-CoV-1, and SARS-CoV-2.

The multivalent antibody contains at least one polypeptide chain (and preferably two polypeptide chains), wherein the polypeptide chain or chains comprise two or more variable domains. For instance, the polypeptide chain or chains may comprise VD1-(X1)ₙ-VD2-(X2)ₙ-Fc, wherein VD1 is a first variable domain, VD2 is a second variable domain, Fc is one polypeptide chain of an Fc region, X1 and X2 represent an amino acid or polypeptide, and n is 0 or 1. Similarly, the polypeptide chain or chains may comprise V_{H}-C_{H}1-flexible linker-V_{H}-C_{H}1-Fc region chain; or V_{H}-C_{H}1-V_{H}-C_{H}1-Fc region chain. The multivalent antibody herein preferably further comprises at least two (and preferably four) light chain variable domain polypeptides. The multivalent antibody herein may, for instance, comprise from about two to about eight light chain variable domain polypeptides. The light chain variable domain polypeptides contemplated here comprise a light chain variable domain and, optionally, further comprise a CL domain.

Techniques for making multispecific antibodies include, but are not limited to, recombinant co-expression of two immunoglobulin heavy chain- light chain pairs having different specificities (see Milstein and Cuello Nature 305: 537 (1983), WO 93/08829, and Traunecker et al. EMBO J. 10:3655 (1991)), and “knob-in-hole” engineering (see, e.g., U.S. Pat. No. 5,731,168). See also WO 2013/026833 (CrossMab). Multi-specific antibodies may also be made by engineering electrostatic steering effects for making antibody Fc-heterodimeric molecules (WO 2009/089004A1); cross-linking two or more antibodies (see, e.g., U.S. Pat. No. 4,676,980); using leucine; using “diabody” technology for making bispecific antibody fragments (see, e.g., Hollinger et al. Proc. Natl. Acad. Sci. USA 90:6444-6448 (1993)); and using single-chain Fv (scFv) dimers (see, e.g., Gruber et al. J. Immunol. 152:5368 (1994)); and preparing trispecific antibodies as described, e.g., in Tutt et al. J. Immunol. 147: 60 (1991).

Engineered antibodies with three or more functional antigen binding sites, including “Octopus antibodies,” are also included herein (see, e.g., US 2006/0025576). The antibody herein also includes a “Dual Acting FAb” or “DAF” comprising an antigen binding site that binds to multiple TMEM106B (see, US 2008/0069820, for example).

### (7) Antibody Variants

In some embodiments of any of the antibodies provided herein, amino acid sequence variants of the antibodies are contemplated. For example, it may be desirable to improve the binding affinity and/or other biological properties of the antibody.

### Substitution, Insertion, and Deletion Variants

In some embodiments of any of the antibodies provided herein, antibody variants having one or more amino acid substitutions are provided. Amino acid sequence variants of an antibody may be prepared by introducing appropriate modifications into the nucleotide sequence encoding the antibody, or by peptide synthesis. Such modifications include, for example, deletions from, and/or insertions into and/or substitutions of residues within the amino acid sequences of the antibody.

TABLE A
Amino Acid Substitutions

| Original | | Preferred |
|---|---|---|
| Residue | Exemplary Substitutions | Substitutions |
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe; Norleucine | Leu |
| Leu (L) | Norleucine; Ile; Val; Met; Ala; Phe | Ile |
| Lys (K) | Arg; Gln; Asn | Arg |
| Met (M) | Leu; Phe; Ile | Leu |
| Phe (F) | Leu; Val; Ile; Ala; Tyr | Tyr |
| Pro (P) | Ala | Ala |
| Ser (S) | Thr | Thr |
| Thr (T) | Ser | Ser |
| Trp (W) | Tyr; Phe | Tyr |
| Tyr (Y) | Trp; Phe; Thr; Ser | Phe |
| Val (V) | Ile; Leu; Met; Phe; Ala; Norleucine | Leu |

Substantial modifications in the biological properties of the antibody are accomplished by selecting substitutions that differ significantly in their effect on maintaining (a) the structure of the polypeptide backbone in the area of the substitution, for example, as a sheet or helical conformation, (b) the charge or hydrophobicity of the molecule at the target site, or (c) the bulk of the side chain. Naturally occurring residues are divided into groups based on common side-chain properties:
(1) hydrophobic: Norleucine, Met, Ala, Val, Leu, Ile;
(2) neutral hydrophilic: Cys, Ser, Thr, Asn, Gln;
(3) acidic: Asp, Glu;
(4) basic: His, Lys, Arg;
(5) residues that influence chain orientation: Gly, Pro; and
(6) aromatic: Trp, Tyr, Phe.

For example, non-conservative substitutions can involve the exchange of a member of one of these classes for a member from another class. Such substituted residues can be introduced, for example, into regions of a human antibody that are homologous with non-human antibodies, or into the non-homologous regions of the molecule.

In making changes to the polypeptide or antibody described herein, according to certain embodiments, the hydropathic index of amino acids can be considered. Each amino acid has been assigned a hydropathic index on the basis of its hydrophobicity and charge characteristics. They are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

The importance of the hydropathic amino acid index in conferring interactive biological function on a protein is understood in the art. Kyte et al. J. Mol. Biol., 157:105-131 (1982). It is known that certain amino acids can be substituted for other amino acids having a similar hydropathic index or score and still retain a similar biological activity. In making changes based upon the hydropathic index, in certain embodiments, the substitution of amino acids whose hydropathic indices are within ±2 is included.

In certain embodiments, those which are within ±1 are included, and in certain embodiments, those within ±0.5 are included.

It is also understood in the art that the substitution of like amino acids can be made effectively on the basis of hydrophilicity, particularly where the biologically functional protein or peptide thereby created is intended for use in immunological embodiments, as in the present case. In certain embodiments, the greatest local average hydrophilicity of a protein, as governed by the hydrophilicity of its adjacent amino acids, correlates with its immunogenicity and antigenicity, i.e., with a biological property of the protein.

The following hydrophilicity values have been assigned to these amino acid residues: arginine (+3.0); lysine (+3.0±1); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5) and tryptophan (−3.4). In making changes based upon similar hydrophilicity values, in certain embodiments, the substitution of amino acids whose hydrophilicity values are within ±2 is included, in certain embodiments, those which are within ±1 are included, and in certain embodiments, those within ±0.5 are included. One can also identify epitopes from primary amino acid sequences on the basis of hydrophilicity. These regions are also referred to as “epitopic core regions”.

In certain embodiments, substitutions, insertions, or deletions may occur within one or more HVRs so long as such alterations do not substantially reduce the ability of the antibody to bind antigen. For example, conservative alterations (e.g., conservative substitutions as provided herein) that do not substantially reduce binding affinity may be made in HVRs. Such alterations may, for example, be outside of antigen contacting residues in the HVRs. In certain embodiments of the variant V_{H} and V_{L} sequences provided above, each HVR either is unaltered, or contains no more than one, two or three amino acid substitutions.

Amino acid sequence insertions include amino- and/or carboxyl-terminal fusions ranging in length from one residue to polypeptides comprising a hundred or more residues, as well as intrasequence insertions of single or multiple amino acid residues. Examples of terminal insertions include an antibody with an N-terminal methionyl residue. Other insertional variants of the antibody molecule include the fusion to the N- or C-terminus of the antibody to an enzyme (e.g., for ADEPT) or a polypeptide which increases the serum half-life of the antibody.

Any cysteine residue not involved in maintaining the proper conformation of the antibody also may be substituted, generally with serine, to improve the oxidative stability of the molecule and prevent aberrant crosslinking. Conversely, cysteine bond(s) may be added to the antibody to improve its stability (particularly where the antibody is an antibody fragment, such as an Fv fragment).

### (ii) Glycosylation Variants

In some embodiments of any of the antibodies provided herein, the antibody is altered to increase or decrease the extent to which the antibody is glycosylated. Addition or deletion of glycosylation sites to an antibody may be conveniently accomplished by altering the amino acid sequence such that one or more glycosylation sites is created or removed.

Glycosylation of antibodies is typically either N-linked or O-linked. N-linked refers to the attachment of the carbohydrate moiety to the side chain of an asparagine residue. The tripeptide sequences asparagine-X-serine and asparagine-X-threonine, where X is any amino acid except proline, are the recognition sequences for enzymatic attachment of the carbohydrate moiety to the asparagine side chain. Thus, the presence of either of these tripeptide sequences in a polypeptide creates a potential glycosylation site. O-linked glycosylation refers to the attachment of one of the sugars N-aceylgalactosamine, galactose, or xylose to a hydroxyamino acid, most commonly serine or threonine, although 5-hydroxyproline or 5-hydroxylysine may also be used.

Addition of glycosylation sites to the antibody is conveniently accomplished by altering the amino acid sequence such that it contains one or more of the above-described tripeptide sequences (for N-linked glycosylation sites). The alteration may also be made by the addition of, or substitution by, one or more serine or threonine residues to the sequence of the original antibody (for O-linked glycosylation sites).

Where the antibody comprises an Fc region, the carbohydrate attached thereto may be altered. Native antibodies produced by mammalian cells typically comprise a branched, biantennary oligosaccharide that is generally attached by an N-linkage to Asn297 according to Kabat numbering of the CH2 domain of the Fc region. The oligosaccharide may include various carbohydrates, for example, mannose, N-acetyl glucosamine (GlcNAc), galactose, and sialic acid, as well as a fucose attached to a GlcNAc in the “stem” of the biantennary oligosaccharide structure. In some embodiments, modifications of the oligosaccharide in an antibody of the invention may be made in order to create antibody variants with certain improved properties.

In one embodiment, antibody variants are provided having a carbohydrate structure that lacks fucose attached (directly or indirectly) to an Fc region. See, e.g., US Patent Publication Nos. 2003/0157108 and 2004/0093621. Examples of publications related to “defucosylated” or “fucose-deficient” antibody variants include: US 2003/0157108; US 2003/0115614; US 2002/0164328; US 2004/0093621; US 2004/0132140; US 2004/0110704; US 2004/0110282; US 2004/0109865; Okazaki et al. J. Mol. Biol. 336:1239-1249 (2004); Yamane-Ohnuki et al. Biotech. Bioeng. 87:614 (2004). Examples of cell lines capable of producing defucosylated antibodies include Led 3 CHO cells deficient in protein fucosylation (Ripka et al. Arch. Biochem. Biophys. 249:533-545 (1986); US 2003/0157108), and knockout cell lines, such as alpha-1,6-fucosyltransferase gene, FUT8, knockout CHO cells (see, e.g., Yamane-Ohnuki et al. Biotech. Bioeng. 87: 614 (2004) and Kanda et al. Biotechnol. Bioeng. 94(4):680-688 (2006)).

(iii) Modified Constant Regions

In some embodiments of any of the antibodies provided herein, the antibody Fc is an antibody Fc isotypes and/or modifications. In some embodiments, the antibody Fc isotype and/or modification is capable of binding to Fc gamma receptor.

In some embodiments of any of the antibodies provided herein, the modified antibody Fc is an IgG1 modified Fc. In some embodiments, the IgG1 modified Fc comprises one or more modifications. For example, in some embodiments, the IgG1 modified Fc comprises one or more amino acid substitutions (e.g., relative to a wild-type Fc region of the same isotype). In some embodiments, the one or more amino acid substitutions are selected from N297A (Bolt S et al. (1993) Eur J Immunol 23:403-411), D265A (Shields et al. (2001) R. I Biol. Chem. 276, 6591-6604), L234A, L235A (Hutchins et al. (1995) Proc Natl Acad Sci USA, 92:11980-11984; Alegre et al., (1994) Transplantation 57:1537-1543. 31; Xu et al., (2000) Cell Immunol, 200:16-26), G237A (Alegre et al. (1994) Transplantation 57:1537-1543. 31; Xu et al. (2000) Cell Immunol, 200:16-26), C226S, C229S, E233P, L234V, L234F, L235E (McEarchern et al., (2007) Blood, 109:1185-1192), P331S (Sazinsky et al., (2008) Proc Natl Acad Sci USA 2008, 105:20167-20172), S267E, L328F, A330L, M252Y, S254T, and/or T256E, where the amino acid position is according to the EU numbering convention.

In some embodiments of any of the IgG1 modified Fc, the Fc comprises N297A mutation according to EU numbering. In some embodiments of any of the IgG1 modified Fc, the Fc comprises D265A and N297A mutations according to EU numbering. In some embodiments of any of the IgG1 modified Fc, the Fc comprises D270A mutations according to EU numbering. In some embodiments, the IgG1 modified Fc comprises L234A and L235A mutations according to EU numbering. In some embodiments of any of the IgG1 modified Fc, the Fc comprises L234A and G237A mutations according to EU numbering. In some embodiments of any of the IgG1 modified Fc, the Fc comprises L234A, L235A and G237A mutations according to EU numbering. In some embodiments of any of the IgG1 modified Fc, the Fc comprises one or more (including all) of P238D, L328E, E233, G237D, H268D, P271G and A330R mutations according to EU numbering. In some embodiments of any of the IgG1 modified Fc, the Fc comprises one or more of S267E/L328F mutations according to EU numbering. In some embodiments of any of the IgG1 modified Fc, the Fc comprises P238D, L328E, E233D, G237D, H268D, P271G and A330R mutations according to EU numbering. In some embodiments of any of the IgG1 modified Fc, the Fc comprises P238D, L328E, G237D, H268D, P271G and A330R mutations according to EU numbering. In some embodiments of any of the IgG1 modified Fc, the Fc comprises P238D, S267E, L328E, E233D, G237D, H268D, P271G and A330R mutations according to EU numbering. In some embodiments of any of the IgG1 modified Fc, the Fc comprises P238D, S267E, L328E, G237D, H268D, P271G and A330R mutations according to EU numbering. In some embodiments of any of the IgG1 modified Fc, the Fc comprises C226S, C229S, E233P, L234V, and L235A mutations according to EU numbering. In some embodiments of any of the IgG1 modified Fc, the Fc comprises L234F, L235E, and P331S mutations according to EU numbering. In some embodiments of any of the IgG1 modified Fc, the Fc comprises S267E and L328F mutations according to EU numbering. In some embodiments of any of the IgG1 modified Fc, the Fc comprises S267E mutations according to EU numbering. In some embodiments of any of the IgG1 modified Fc, the Fc comprises a substitute of the constant heavy 1 (CH1) and hinge region of IgG1 with C_{H}1 and hinge region of IgG2 (amino acids 118-230 of IgG2 according to EU numbering) with a Kappa light chain.

In some embodiments of any of the IgG1 modified Fc, the Fc includes two or more amino acid substitutions that increase antibody clustering without activating complement as compared to a corresponding antibody having an Fc region that does not include the two or more amino acid substitutions. Accordingly, in some embodiments of any of the IgG1 modified Fc, the IgG1 modified Fc is an antibody comprising an Fc region, where the antibody comprises an amino acid substitution at position E430G and one or more amino acid substitutions in the Fc region at a residue position selected from: L234F, L235A, L235E, S267E, K322A, L328F, A330S, P331S, and any combination thereof according to EU numbering. In some embodiments, the IgG1 modified Fc comprises an amino acid substitution at positions E430G, L243A, L235A, and P331S according to EU numbering. In some embodiments, the IgG1 modified Fc comprises an amino acid substitution at positions E430G and P331S according to EU numbering. In some embodiments, the IgG1 modified Fc comprises an amino acid substitution at positions E430G and K322A according to EU numbering. In some embodiments, the IgG1 modified Fc comprises an amino acid substitution at positions E430G, A330S, and P331S according to EU numbering. In some embodiments, the IgG1 modified Fc comprises an amino acid substitution at positions E430G, K322A, A330S, and P331S according to EU numbering. In some embodiments, the IgG1 modified Fc comprises an amino acid substitution at positions E430G, K322A, and A330S according to EU numbering. In some embodiments, the IgG1 modified Fc comprises an amino acid substitution at positions E430G, K322A, and P331S according to EU numbering.

In some embodiments of any of the IgG1 modified Fc, the IgG1 modified Fc may further comprise herein may be combined with an A330L mutation (Lazar et al. Proc Natl Acad Sci USA, 103:4005-4010 (2006)), or one or more of L234F, L235E, and/or P331S mutations (Sazinsky et al. Proc Natl Acad Sci USA, 105:20167-20172 (2008)), according to the EU numbering convention, to eliminate complement activation. In some embodiments of any of the IgG1 modified Fc, the IgG1 modified Fc may further comprise one or more of A330L, A330S, L234F, L235E, and/or P331S according to EU numbering. In some embodiments of any of the IgG1 modified Fc, the IgG1 modified Fc may further comprise one or more mutations to enhance the antibody half-life in human serum (e.g., one or more (including all) of M252Y, S254T, and T256E mutations according to the EU numbering convention). In some embodiments of any of the IgG1 modified Fc, the IgG1 modified Fc may further comprise one or more of E430G, E430S, E430F, E430T, E345K, E345Q, E345R, E345Y, S440Y, and/or S440W according to EU numbering.

Other aspects of the present disclosure relate to antibodies having modified constant regions (i.e., Fc regions). An antibody dependent on binding to FcgR receptor to activate targeted receptors may lose its agonist activity if engineered to eliminate FcgR binding (see, e.g., Wilson et al. Cancer Cell 19:101-113 (2011); Armour at al. Immunology 40:585-593 (2003); and White et al. Cancer Cell 27:138-148 (2015)). As such, it is thought that an anti-TMEM106B antibody of the present disclosure with the correct epitope specificity can activate the target antigen, with minimal adverse effects, when the antibody has an Fc domain from a human IgG2 isotype (C_{H}1 and hinge region) or another type of Fc domain that is capable of preferentially binding the inhibitory FcgRIIB r receptors, or a variation thereof.

In some embodiments of any of the antibodies provided herein, the modified antibody Fc is an IgG2 modified Fc. In some embodiments, the IgG2 modified Fc comprises one or more modifications. For example, in some embodiments, the IgG2 modified Fc comprises one or more amino acid substitutions (e.g., relative to a wild-type Fc region of the same isotype). In some embodiments of any of the IgG2 modified Fc, the one or more amino acid substitutions are selected from V234A (Alegre et al. Transplantation 57:1537-1543 (1994); Xu et al. Cell Immunol, 200:16-26 (2000)); G237A (Cole et al. Transplantation, 68:563-571 (1999)); H268Q, V309L, A330S, P331S (US 2007/0148167; Armour et al. Eur J Immunol 29: 2613-2624 (1999); Armour et al. The Haematology Journal 1(Supp1.1):27 (2000); Armour et al. The Haematology Journal 1(Supp1.1):27 (2000)), C219S, and/or C220S (White et al. Cancer Cell 27, 138-148 (2015)); S267E, L328F (Chu et al. Mol Immunol, 45:3926-3933 (2008)); and M252Y, S254T, and/or T256E according to the EU numbering convention. In some embodiments of any of the IgG2 modified Fc, the Fc comprises an amino acid substitution at positions V234A and G237A according to EU numbering. In some embodiments of any of the IgG2 modified Fc, the Fc comprises an amino acid substitution at positions C219S or C220S according to EU numbering. In some embodiments of any of the IgG2 modified Fc, the Fc comprises an amino acid substitution at positions A330S and P331S according to EU numbering. In some embodiments of any of the IgG2 modified Fc, the Fc comprises an amino acid substitution at positions S267E and L328F according to EU numbering.

In some embodiments of any of the IgG2 modified Fc, the Fc comprises a C127S amino acid substitution according to the EU numbering convention (White et al., (2015) Cancer Cell 27, 138-148; Lightle et al. Protein Sci. 19:753-762 (2010); and WO 2008/079246). In some embodiments of any of the IgG2 modified Fc, the antibody has an IgG2 isotype with a Kappa light chain constant domain that comprises a C214S amino acid substitution according to the EU numbering convention (White et al. Cancer Cell 27:138-148 (2015); Lightle et al. Protein Sci. 19:753-762 (2010); and WO 2008/079246).

In some embodiments of any of the IgG2 modified Fc, the Fc comprises a C220S amino acid substitution according to the EU numbering convention. In some embodiments of any of the IgG2 modified Fc, the antibody has an IgG2 isotype with a Kappa light chain constant domain that comprises a C214S amino acid substitution according to the EU numbering convention.

In some embodiments of any of the IgG2 modified Fc, the Fc comprises a C219S amino acid substitution according to the EU numbering convention. In some embodiments of any of the IgG2 modified Fc, the antibody has an IgG2 isotype with a Kappa light chain constant domain that comprises a C214S amino acid substitution according to the EU numbering convention.

In some embodiments of any of the IgG2 modified Fc, the Fc includes an IgG2 isotype heavy chain constant domain 1(CH1) and hinge region (White et al. Cancer Cell 27:138-148 (2015)). In certain embodiments of any of the IgG2 modified Fc, the IgG2 isotype C_{H}1 and hinge region comprise the amino acid sequence of 118-230 according to EU numbering. In some embodiments of any of the IgG2 modified Fc, the antibody Fc region comprises a S267E amino acid substitution, a L328F amino acid substitution, or both, and/or a N297A or N297Q amino acid substitution according to the EU numbering convention.

In some embodiments of any of the IgG2 modified Fc, the Fc further comprises one or more amino acid substitution at positions E430G, E430S, E430F, E430T, E345K, E345Q, E345R, E345Y, S440Y, and S440W according to EU numbering. In some embodiments of any of the IgG2 modified Fc, the Fc may further comprise one or more mutations to enhance the antibody half-life in human serum (e.g., one or more (including all) of M252Y, S254T, and T256E mutations according to the EU numbering convention). In some embodiments of any of the IgG2 modified Fc, the Fc may further comprise A330S and P331S.

In some embodiments of any of the IgG2 modified Fc, the Fc is an IgG2/4 hybrid Fc. In some embodiments, the IgG2/4 hybrid Fc comprises IgG2 aa 118 to 260 and IgG4 aa 261 to 447. In some embodiments of any IgG2 modified Fc, the Fc comprises one or more amino acid substitutions at positions H268Q, V309L, A330S, and P331S according to EU numbering.

In some embodiments of any of the IgG1 and/or IgG2 modified Fc, the Fc comprises one or more additional amino acid substitutions selected from A330L, L234F; L235E, or P331S according to EU numbering; and any combination thereof

In certain embodiments of any of the IgG1 and/or IgG2 modified Fc, the Fc comprises one or more amino acid substitutions at a residue position selected from C127S, L234A, L234F, L235A, L235E, S267E, K322A, L328F, A330S, P331S, E345R, E430G, S440Y, and any combination thereof according to EU numbering. In some embodiments of any of the IgG1 and/or IgG2 modified Fc, the Fc comprises an amino acid substitution at positions E430G, L243A, L235A, and P331S according to EU numbering. In some embodiments of any of the IgG1 and/or IgG2 modified Fc, the Fc comprises an amino acid substitution at positions E430G and P331S according to EU numbering. In some embodiments of any of the IgG1 and/or IgG2 modified Fc, the Fc comprises an amino acid substitution at positions E430G and K322A according to EU numbering. In some embodiments of any of the IgG1 and/or IgG2 modified Fc, the Fc comprises an amino acid substitution at positions E430G, A330S, and P331S according to EU numbering. In some embodiments of any of the IgG1 and/or IgG2 modified Fc, the Fc comprises an amino acid substitution at positions E430G, K322A, A330S, and P331S according to EU numbering. In some embodiments of any of the IgG1 and/or IgG2 modified Fc, the Fc comprises an amino acid substitution at positions E430G, K322A, and A330S according to EU numbering. In some embodiments of any of the IgG1 and/or IgG2 modified Fc, the Fc comprises an amino acid substitution at positions E430G, K322A, and P331S according to EU numbering. In some embodiments of any of the IgG1 and/or IgG2 modified Fc, the Fc comprises an amino acid substitution at positions S267E and L328F according to EU numbering. In some embodiments of any of the IgG1 and/or IgG2 modified Fc, the Fc comprises an amino acid substitution at position C127S according to EU numbering. In some embodiments of any of the IgG1 and/or IgG2 modified Fc, the Fc comprises an amino acid substitution at positions E345R, E430G and S440Y according to EU numbering.

In some embodiments of any of the antibodies provided herein, the modified antibody Fc is an IgG4 modified Fc. In some embodiments, the IgG4 modified Fc comprises one or more modifications. For example, in some embodiments, the IgG4 modified Fc comprises one or more amino acid substitutions (e.g., relative to a wild-type Fc region of the same isotype). In some embodiments of any of the IgG4 modified Fc, the one or more amino acid substitutions are selected from L235A, G237A, S229P, L236E (Reddy et al. J Immunol 164:1925-1933(2000)), S267E, E318A, L328F, M252Y, S254T, and/or T256E according to the EU numbering convention. In some embodiments of any of the IgG4 modified Fc, the Fc may further comprise L235A, G237A, and E318A according to the EU numbering convention. In some embodiments of any of the IgG4 modified Fc, the Fc may further comprise S228P and L235E according to the EU numbering convention. In some embodiments of any of the IgG4 modified Fc, the IgG4 modified Fc may further comprise S267E and L328F according to the EU numbering convention.

In some embodiments of any of the IgG4 modified Fc, the IgG4 modified Fc comprises may be combined with an S228P mutation according to the EU numbering convention (Angal et al. Mol Immunol. 30:105-108 (1993)) and/or with one or more mutations described in (Peters et al. J Biol Chem. 287(29):24525-33 (2012)) to enhance antibody stabilization.

In some embodiments of any of the IgG4 modified Fc, the IgG4 modified Fc may further comprise one or more mutations to enhance the antibody half-life in human serum (e.g., one or more (including all) of M252Y, S254T, and T256E mutations according to the EU numbering convention).

In some embodiments of any of the IgG4 modified Fc, the Fc comprises L235E according to EU numbering. In certain embodiments of any of the IgG4 modified Fc, the Fc comprises one or more amino acid substitutions at a residue position selected from C127S, F234A, L235A, L235E, S267E, K322A, L328F, E345R, E430G, S440Y, and any combination thereof, according to EU numbering. In some embodiments of any of the IgG4 modified Fc, the Fc comprises an amino acid substitution at positions E430G, L243A, L235A, and P331S according to EU numbering. In some embodiments of any of the IgG4 modified Fc, the Fc comprises an amino acid substitution at positions E430G and P331S according to EU numbering. In some embodiments of any of the IgG4 modified Fc, the Fc comprises an amino acid substitution at positions E430G and K322A according to EU numbering. In some embodiments of any of the IgG4 modified Fc, the Fc comprises an amino acid substitution at position E430 according to EU numbering. In some embodiments of any of the IgG4 modified Fc, the Fc region comprises an amino acid substitution at positions E430G and K322A according to EU numbering. In some embodiments of any of the IgG4 modified Fc, the Fc comprises an amino acid substitution at positions S267E and L328F according to EU numbering. In some embodiments of any of the IgG4 modified Fc, the Fc comprises an amino acid substitution at position C127S according to EU numbering. In some embodiments of any of the IgG4 modified Fc, the Fc comprises an amino acid substitution at positions E345R, E430G and S440Y according to EU numbering.

### (8) Other Antibody Modifications

In some embodiments of any of the antibodies, the antibody is a derivative. The term “derivative” refers to a molecule that includes a chemical modification other than an insertion, deletion, or substitution of amino acids (or nucleic acids). In certain embodiments, derivatives comprise covalent modifications, including, but not limited to, chemical bonding with polymers, lipids, or other organic or inorganic moieties. In certain embodiments, a chemically modified antigen binding protein can have a greater circulating half-life than an antigen binding protein that is not chemically modified. In certain embodiments, a chemically modified antigen binding protein can have improved targeting capacity for desired cells, tissues, and/or organs. In some embodiments, a derivative antigen binding protein is covalently modified to include one or more water soluble polymer attachments, including, but not limited to, polyethylene glycol, polyoxyethylene glycol, or polypropylene glycol. See, e.g., U.S. Pat. Nos. 4,640,835, 4,496,689, 4,301,144, 4,670,417, 4,791192 and 4,,179,337. In certain embodiments, a derivative antigen binding protein comprises one or more polymer, including, but not limited to, monomethoxy-polyethylene glycol, dextran, cellulose, copolymers of ethylene glycol/propylene glycol, carboxymethylcellulose, polyvinyl pyrrolidone, poly-1, 3-dioxolane, poly-1,3,6-trioxane, ethylene/maleic anhydride copolymer, polyaminoacids (either homopolymers or random copolymers), poly-(N-vinyl pyrrolidone)-polyethylene glycol, propylene glycol homopolymers, a polypropylene oxide/ethylene oxide co-polymer, polyoxyethylated polyols (e.g., glycerol) and polyvinyl alcohol, as well as mixtures of such polymers.

In certain embodiments, a derivative is covalently modified with polyethylene glycol (PEG) subunits. In certain embodiments, one or more water-soluble polymer is bonded at one or more specific position, for example at the amino terminus, of a derivative. In certain embodiments, one or more water-soluble polymer is randomly attached to one or more side chains of a derivative. In certain embodiments, PEG is used to improve the therapeutic capacity for an antigen binding protein. In certain embodiments, PEG is used to improve the therapeutic capacity for a humanized antibody. Certain such methods are discussed, for example, in U.S. Pat. No. 6,133,426, which is hereby incorporated by reference for any purpose.

Peptide analogs are commonly used in the pharmaceutical industry as non-peptide drugs with properties analogous to those of the template peptide. These types of non-peptide compound are termed “peptide mimetics” or “peptidomimetics.” Fauchere, J. Adv. Drug Res., 15:29 (1986); and Evans et al. J. Med. Chem., 30:1229 (1987), which are incorporated herein by reference for any purpose. Such compounds are often developed with the aid of computerized molecular modeling. Peptide mimetics that are structurally similar to therapeutically useful peptides can be used to produce a similar therapeutic or prophylactic effect. Generally, peptidomimetics are structurally similar to a paradigm polypeptide (i.e., a polypeptide that has a biochemical property or pharmacological activity), such as human antibody, but have one or more peptide linkages optionally replaced by a linkage selected from: —CH₂NH—, —CH₂S—, —CH₂—CH₂—, —CH═CH-(cis and trans), —COCH₂—, —CH(OH)CH₂—, and —CH₂SO—, by methods well known in the art. Systematic substitution of one or more amino acids of a consensus sequence with a D-amino acid of the same type (e.g., D-lysine in place of L-lysine) can be used in certain embodiments to generate more stable peptides. In addition, constrained peptides comprising a consensus sequence or a substantially identical consensus sequence variation can be generated by methods known in the art (Rizo and Gierasch Ann. Rev. Biochem., 61:387 (1992), incorporated herein by reference for any purpose); for example, by adding internal cysteine residues capable of forming intramolecular disulfide bridges which cyclize the peptide. 101811 Drug conjugation involves coupling of a biological active cytotoxic (anticancer) payload or drug to an antibody that specifically targets a certain tumor marker (e.g. a polypeptide that, ideally, is only to be found in or on tumor cells). Antibodies track these proteins down in the body and attach themselves to the surface of cancer cells. The biochemical reaction between the antibody and the target protein (antigen) triggers a signal in the tumor cell, which then absorbs or internalizes the antibody together with the cytotoxin. After the ADC is internalized, the cytotoxic drug is released and kills the cancer. Due to this targeting, ideally the drug has lower side effects and gives a wider therapeutic window than other chemotherapeutic agents. Technics to conjugate antibodies are disclosed are known in the art (see, e.g., Jane de Lartigue OncLive Jul. 5, 2012; ADC Review on antibody-drug conjugates; and Duch et al. Bioconjugate Chemistry 21 (1):5-13 (2010).

### Nucleic Acids, Vectors, and Host Cells

Anti-TMEM106B antibodies of the present disclosure may be produced using recombinant methods and compositions, e.g., as described in U.S. Pat. No. 4,816,567. In some embodiments, isolated nucleic acids having a nucleotide sequence encoding any of the anti-TMEM106B antibodies of the present disclosure are provided. Such nucleic acids may encode an amino acid sequence comprising the V_{L} and/or an amino acid sequence comprising the V H of the anti-TMEM106B antibody (e.g., the light and/or heavy chains of the antibody). In some embodiments, one or more vectors (e.g., expression vectors) comprising such nucleic acids are provided. In some embodiments, a host cell comprising such nucleic acid is also provided. In some embodiments, the host cell comprises (e.g., has been transduced with): (1) a vector comprising a nucleic acid that encodes an amino acid sequence comprising the V_{L} of the antibody and an amino acid sequence comprising the V_{H} of the antibody, or (2) a first vector comprising a nucleic acid that encodes an amino acid sequence comprising the V_{L} of the antibody and a second vector comprising a nucleic acid that encodes an amino acid sequence comprising the V_{H} of the antibody. In some embodiments, the host cell is eukaryotic, e.g., a Chinese Hamster Ovary (CHO) cell or lymphoid cell (e.g., Y0, NS0, Sp20 cell). Host cells of the present disclosure also include, without limitation, isolated cells, in vitro cultured cells, and ex vivo cultured cells.

Methods of making an anti-TMEM106B antibody of the present disclosure are provided. In some embodiments, the method includes culturing a host cell of the present disclosure comprising a nucleic acid encoding the anti-TMEM106B antibody, under conditions suitable for expression of the antibody. In some embodiments, the antibody is subsequently recovered from the host cell (or host cell culture medium).

For recombinant production of an anti-TMEM106B antibody of the present disclosure, a nucleic acid encoding the anti-TMEM106B antibody is isolated and inserted into one or more vectors for further cloning and/or expression in a host cell. Such nucleic acid may be readily isolated and sequenced using conventional procedures (e.g., by using oligonucleotide probes that are capable of binding specifically to genes encoding the heavy and light chains of the antibody).

Suitable vectors comprising a nucleic acid sequence encoding any of the anti-TMEM106B antibodies of the present disclosure, or cell-surface expressed fragments or polypeptides thereof polypeptides (including antibodies) described herein include, without limitation, cloning vectors and expression vectors. Suitable cloning vectors can be constructed according to standard techniques, or may be selected from a large number of cloning vectors available in the art. While the cloning vector selected may vary according to the host cell intended to be used, useful cloning vectors generally have the ability to self-replicate, may possess a single target for a particular restriction endonuclease, and/or may carry genes for a marker that can be used in selecting clones comprising the vector. Suitable examples include plasmids and bacterial viruses, e.g., pUC18, pUC19, Bluescript (e.g., pBS SK+) and its derivatives, mp18, mp19, pBR322, pMB9, ColEl, pCR1, RP4, phage DNAs, and shuttle vectors such as pSA3 and pAT28. These and many other cloning vectors are available from commercial vendors such as BioRad, Strategene, and Invitrogen.

Suitable host cells for cloning or expression of antibody-encoding vectors include prokaryotic or eukaryotic cells. For example, anti-TMEM106B antibodies of the present disclosure may be produced in bacteria, in particular when glycosylation and Fc effector function are not needed. For expression of antibody fragments and polypeptides in bacteria (e.g., U.S. Pat. Nos. 5,648,237, 5,789,199, and 5,840,523. After expression, the antibody may be isolated from the bacterial cell paste in a soluble fraction and can be further purified.

In addition to prokaryotes, eukaryotic microorganisms, such as filamentous fungi or yeast, are also suitable cloning or expression hosts for antibody-encoding vectors, including fungi and yeast strains whose glycosylation pathways have been “humanized,” resulting in the production of an antibody with a partially or fully human glycosylation pattern (e.g., Gerngross Nat. Biotech. 22:1409-1414 (2004); and Li et al. Nat. Biotech. 24:210-215 (2006)).

Suitable host cells for the expression of glycosylated antibody can also be derived from multicellular organisms (invertebrates and vertebrates). Examples of invertebrate cells include plant and insect cells. Numerous baculoviral strains have been identified which may be used in conjunction with insect cells, particularly for transfection of Spodoptera frugiperda cells. Plant cell cultures can also be utilized as hosts (e.g., U.S. Pat. Nos. 5,959,177, 6,040,498, 6,420,548, 7,125,978, and 6,417,429, describing PLANTIBODIES TM technology for producing antibodies in transgenic plants).

Vertebrate cells may also be used as hosts. For example, mammalian cell lines that are adapted to grow in suspension may be useful. Other examples of useful mammalian host cell lines are monkey kidney CV1 line transformed by SV40 (COS-7); human embryonic kidney line (293 or 293 cells as described, e.g., in Graham et al. J. Gen Virol. 36:59 (1977)); baby hamster kidney cells (BHK); mouse sertoli cells (TM4 cells as described, e.g., in Mather, Biol. Reprod. 23:243-251 (1980)); monkey kidney cells (CV1); African green monkey kidney cells (VERO-76); human cervical carcinoma cells (HELA); canine kidney cells (MDCK; buffalo rat liver cells (BRL 3A); human lung cells (W138); human liver cells (Hep G2); mouse mammary tumor (MMT 060562); TRI cells, as described, e.g., in Mather et al. Annals N.Y. Acad. Sci. 383:44-68 (1982); MRC 5 cells; and FS4 cells. Other useful mammalian host cell lines include Chinese hamster ovary (CHO) cells, including DHFR- CHO cells (Urlaub et al.Proc. Natl. Acad. Sci. USA 77:4216 (1980)); and myeloma cell lines such as Y0, NS0 and Sp2/0. For a review of certain mammalian host cell lines suitable for antibody production, see, e.g., Yazaki and Wu, Methods in Molecular Biology, Vol. 248 (B.K.C. Lo, ed., Humana Press, Totowa, NJ), pp. 255-268 (2003).

### Pharmaceutical Compositions/Formulations

Provided herein are pharmaceutical compositions and/or pharmaceutical formulations comprising the anti-TMEM106B antibodies of the present disclosure and a pharmaceutically acceptable carrier for use, e.g., in treating, preventing, or reducing the risk of coronavirus infection.

In some aspects, the antibody or antigen-binding fragment thereof having the desired degree of purity is present in a formulation comprising, e.g., a physiologically acceptable carrier, excipient or stabilizer (Remington's Pharmaceutical Sciences (1990) Mack Publishing Co., Easton, PA). In some embodiments, pharmaceutically acceptable carriers preferably are nontoxic to recipients at the dosages and concentrations employed.

Formulations suitable for parenteral administration include aqueous and non-aqueous, isotonic sterile injection solutions, which can comprise antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives.

In some aspects, a pharmaceutical composition comprises an anti-TMEM106B antibody or antigen-binding fragment thereof as described herein, and a pharmaceutically acceptable carrier (see, e.g., Gennaro, Remington: The Science and Practice of Pharmacy with Facts and Comparisons: Drugfacts Plus, 20th ed. (2003); Ansel et al., Pharmaceutical Dosage Forms and Drug Delivery Systems, 7th ed., Lippencott Williams and Wilkins (2004); Kibbe et al., Handbook of Pharmaceutical Excipients, 3rd ed., Pharmaceutical Press (2000)). Pharmaceutical compositions described herein are, in some aspects, for use as a medicament. The compositions to be used for in vivo administration can be sterile. This is readily accomplished by filtration through, e.g., sterile filtration membranes.

A pharmaceutical composition described herein can be used to exert a biological effect(s), e.g., treating, preventing, or reducing the risk of coronavirus infection, in vivo.

### Therapeutic Uses

In some embodiments, the present disclosure provides methods for treating, preventing, or reducing risk of coronavirus infection by administering to an individual in need thereof a therapeutically effective amount of an antibody that binds to TMEM106B protein. In some embodiments, the coronavirus infection is SARS-CoV-2 coronavirus infection and the methods provided herein are effective at treating, preventing, or reducing the risk of SARS-CoV-2 coronavirus infection.

In some embodiments, the present disclosure provides methods for preventing or reducing coronavirus transmission, wherein the method comprises administering to an individual in need thereof a therapeutically effective amount of an antibody that binds to TMEM106B protein, thereby preventing or reducing coronavirus transmission. In some embodiments, the coronavirus is SARS-CoV-2 coronavirus and the methods provided herein are effective at preventing or reducing SARS-CoV-2 coronavirus transmission.

Anti-TMEM106B antibodies of the present disclosure are effective at treating coronavirus infection over a range of clinical manifestations of coronavirus infection, including asymptomatic or presymptomatic infection, mild illness, moderate illness, severe illness, and critical illness.

In some embodiments, a subject or individual is a mammal. Mammals include, without limitation, domesticated animals (e.g., cows, sheep, cats, dogs, and horses), primates (e.g., humans and non-human primates such as monkeys), rabbits, and rodents (e.g., mice and rats). In some embodiments, the subject or individual is a human.

An antibody provided herein (and any additional therapeutic agent) can be administered by any suitable means, including parenteral, intrapulmonary, intranasal, intralesional administration, intracerobrospinal, intracranial, intraspinal, intrasynovial, intrathecal, oral, topical, or inhalation routes. Parenteral infusions include intramuscular, intravenous administration as a bolus or by continuous infusion over a period of time, intraarterial, intra-articular, intraperitoneal, or subcutaneous administration. In some embodiments, the administration is intravenous administration. In some embodiments, the administration is subcutaneous. Dosing can be by any suitable route, e.g. by injections, such as intravenous or subcutaneous injections, depending in part on whether the administration is brief or chronic. Various dosing schedules including but not limited to single or multiple administrations over various time-points, bolus administration, and pulse infusion are contemplated herein.

Antibodies provided herein would be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The antibody need not be, but is optionally formulated with one or more agents currently used to prevent or treat the disorder in question. The effective amount of such other agents depends on the amount of antibody present in the formulation, the type of disorder or treatment, and other factors discussed above. These are generally used in the same dosages and with administration routes as described herein, or about from 1 to 99% of the dosages described herein, or in any dosage and by any route that is empirically/clinically determined to be appropriate.

The present disclosure further contemplates the use of an anti-TMEM106B antibody for treating, preventing, or reducing risk of coronavirus infection in combination with other therapies, including, for example, dexamethasone, remdesivir, baricitinib, casirivimab, imdevimab, bamlanivimab, or any combination thereof

In some embodiments, an antibody of the present disclosure reduces a cytopathic effect in a cell infected with SARS-CoV-2, optionally wherein the cell is a VeroE6 cell or a NCI-H1975 cell.

For the prevention or treatment of disease, the appropriate dosage of an antibody of the invention (when used alone or in combination with one or more other additional therapeutic agents) will depend on the type of disease to be treated, the type of antibody, the severity and course of the disease, whether the antibody is administered for preventive or therapeutic purposes, previous therapy, the patient's clinical history and response to the antibody, and the discretion of the attending physician. The antibody is suitably administered to the patient at one time or over a series of treatments.

### Articles of Manufacture

Provided herein are articles of manufacture (e.g., kit) comprising an anti-TMEM106B antibody described herein. Article of manufacture may include one or more containers comprising an antibody described herein. Containers may be any suitable packaging including, but is not limited to, vials, bottles, jars, flexible packaging (e.g., sealed Mylar or plastic bags), and the like. The containers may be unit doses, bulk packages (e.g., multi-dose packages) or sub-unit doses.

In some embodiments, the kits may further include a second agent. In some embodiments, the second agent is a pharmaceutically-acceptable buffer or diluting agent including, but not limited to, such as bacteriostatic water for injection (BWFI), phosphate- buffered saline, Ringer's solution and dextrose solution. In some embodiments, the second agent is a pharmaceutically active agent.

In some embodiments of any of the articles of manufacture, the article of manufactures further include instructions for use in accordance with the methods of this disclosure. The instructions generally include information as to dosage, dosing schedule, and route of administration for the intended treatment. In some embodiments, these instructions comprise a description of administration of the isolated antibody of the present disclosure (e.g., an anti-TMEM106B antibody described herein) to prevent, reduce risk, or treat an individual having a disease, disorder, or injury selected from frontotemporal lobar degeneration, frontotemporal dementia, frontotemporal dementia with progranulin mutations, frontotemporal dementia with C9orf72 mutations, frontotemporal lobar degeneration with TDP-43 inclusions, TDP-43 proteinopathy, hippocampal sclerosis (HpScl), hippocampal sclerosis of aging (HS-Aging), cognitive impairments associated with various disorders (including without limitation cognitive impairment in amyotrophic lateral sclerosis), and hypomyelinating disorder (including without limitation hypomyelinating leukodystrophy), according to any methods of this disclosure. In some embodiments, the instructions include instructions for use of the anti-TMEM106B antibody and the second agent (e.g., second pharmaceutically active agent).

The present disclosure will be more fully understood by reference to the following Examples. They should not, however, be construed as limiting the scope of the present disclosure. All citations throughout the disclosure are hereby expressly incorporated by reference.

### EXAMPLES

### Example 1: Anti-TMEM106B Antibody Identification

Monoclonal antibodies targeting human TMEM106B were generated as previously described (see International Patent Application Publication No. W02019/133512, the content of which is herein incorporated by reference in its entirety).

In brief, NZB/W mice (JAX100008, Jackson Laboratory, Bar Harbor, ME), SJL mice (JAX000686, Jackson Laboratory), or TMEM106B.knockout mice (Taconic, Rensselaer, NY) were co-immunized weekly with 50 μg each of plasmid DNA encoding full-length human, cynomolgus (cyno), or mouse TMEM106B with or without mFlt3 ligand (DNA) and mGM-CSF (DNA) (Invitrogen, San Diego, CA) diluted in lactated Ringer's solution. A total of 5-7 injections of the TMEM106B expression plasmids for DNA immunizations were performed per mouse. Spleens were harvested from the mice three days following the final DNA immunization. Sera from the mice were analyzed for reactivity to TMEM106B by FACS analyses using HEK293 cells overexpressing human, cyno, and/or mouse TMEM106B. Splenocytes from mice whose sera demonstrated strong binding to HEK293 cells overexpressing human, cyno, and/or mouse TMEM106B were fused with P3X63Ag8.653 mouse myeloma cells (CRL-1580, American Type Culture Collection, Rockville, MD) via electrofusion (ECM 2001, BTX, Holliston, MA) and incubated at 37° C./5% CO2 overnight in Clonacell-HY Medium C (StemCell Technologies, Vancouver, BC, Canada). Three rounds of fusions were performed: Fusion A, using splenocytes obtained from immunized TMEM106B.knockout mice; Fusion B, using splenocytes obtained from immunized SJL mice; and Fusion C, using splenocytes obtained from immunized NZB/W mice.

The following day, the fused cells were centrifuged and resuspended in 10 mls of ClonaCell-HY Medium C with anti-mouse IgG Fc-FITC (Jackson Immunoresearch, West Grove, PA) and then gently mixed with 90 mls of methylcellulose-based ClonaCell-HY Medium D (Stemcell Technologies) containing HAT components. The cells were plated into Nunc OmniTrays (Thermo Fisher Scientific, Rochester, NY) and allowed to grow at 37° C./5% CO2 for seven days. Fluorescent colonies were selected and transferred into 96-well plates containing Clonacell-HY Medium E (StemCell Technologies) using a Clonepix 2 (Molecular Devices, Sunnyvale, CA) and screened for TMEM106B reactivity 5 days later.

Initial screening of the anti-TMEM106B hybridomas was performed as follows. Tissue culture supernatants from the hybridomas obtained were initially screened for their ability to differentially bind human TMEM106B transiently-transfected HEK293 cells by comparing the extent of binding to parental (non-transfected) HEK293 cells compared to transfected cells. TMEM106B over-expressing cells were produced via transient transfection of HEK293 cells using the lipofectamine system. To ensure reproducibility across screening experiments, a large bank of transfected cells (1×10⁹) was prepared in a single round of transient transfection, and aliquoted and frozen for all further screening experiments.

For screening of the hybridoma cell culture supernatants, humanTMEM106B-transfected HEK293 cells were aliquoted in 96-well round bottom plates (2×10⁵ cells per well) and incubated with 50 μL of hybridoma cell culture supernatant on ice for 30 minutes. After this primary incubation, the supernatant was removed via centrifugation, the cells were washed twice with 175 μL of ice-cold FACS buffer (PBS+1% FBS+2 mM EDTA), and then further incubated on ice for 20 minutes with anti-mouse IgG Fc-APC (Jackson Labs, Cat# 115-136-071) (diluted 1:500). Following this secondary incubation, the cells were again washed twice with ice-cold FACS buffer and resuspended in a final volume of 30 μμL of FACS buffer+0.25 μl/well propidium iodide (BD Biosciences Cat#556463). Cell sorting was performed on a FACS Canto system (BD Biosciences) or iQue (Intellicyt), with sort gates drawn to exclude dead (i.e., propidium iodide-positive) cells. Median fluorescence intensity (MFI) of anti-mouse-APC MFI on TMEM106B+HEK293 cells was calculated for each clone, and those displaying a signal of at least 2-fold over background compared to a secondary-antibody only well were taken forward for further analysis and characterization.

### Example 2: Antibody Heavy Chain and Light Chain Variable Domain Sequences

Amino acid sequences were determined for anti-TMEM106B antibodies identified as described above. Using standard techniques, the amino acid sequences encoding the light chain variable regions and the heavy chain variable regions of the generated antibodies were determined. The Kabat heavy chain CDR (HVR) amino acid sequences and the Kabat light chain CDR (HVR) amino sequences of the antibodies are set forth below in Table 1, Table 2, Table 3, and Table 4. The amino acid sequences for the heavy chain and light chain variable regions of the anti-TMEM106B antibodies are set forth below in Table 5 and Table 6. In both Table 5 and Table 6, the CDR (HVR) regions, as defined by Kabat, are underlined.

TABLE 1

| Antibody | CDR-H1 | CDR-H2 | CDR-H3 |
|---|---|---|---|
| TM-1 | NYLIE | VINPGSGGTKYNEKLKG | RGYTIYDFY AMDY |
| | (SEQ ID NO: 2) | (SEQ ID NO: 35) | (SEQ ID NO: 78) |
| TM-2 | EYPMH | MIYTNTGEPTYAAEFKG | AGY |
| | (SEQ ID NO: 3) | (SEQ ID NO: 36) | (SEQ ID NO: 79) |
| TM-3 | TLGRGVG | KIWWNDDKFYYPALKS | IAGGTGAAY |
| | (SEQ ID NO: 4) | (SEQ ID NO: 37) | (SEQ ID NO: 80) |
| TM-4 | DYYMN | VINPYNGGTSYHQKFKG | AATVVAGFDY |
| | (SEQ ID NO: 5) | (SEQ ID NO: 38) | (SEQ ID NO: 81) |
| TM-5 | TYGIT | EIYPRSDNTYYNEKFKD | SKGSGTGDY |
| | (SEQ ID NO: 6) | (SEQ ID NO: 39) | (SEQ ID NO: 82) |
| TM-6 | EYTIH | WFYPGSTYIDYNEKFKD | HEEDYSNWFPF |
| | (SEQ ID NO: 7) | (SEQ ID NO: 40) | (SEQ ID NO: 83) |
| TM-7 | DYYIH | LVYPYNGDTDYNQKFKG | TYYANSPDY |
| | (SEQ ID NO: 8) | (SEQ ID NO: 41) | (SEQ ID NO: 84) |
| TM-8 | DYPMH | VIYTDTGEPKYAEVFKG | RLAY |
| | (SEQ ID NO: 9) | (SEQ ID NO: 42) | (SEQ ID NO: 85) |
| TM-9 | GYGMS | TISSGSFYIYYPDSVKG | QNFYYGCEDY |
| | (SEQ ID NO: 10) | (SEQ ID NO: 43) | (SEQ ID NO: 86) |
| TM-10 | SYWMN | QIYPGDGDTNYNGKFKG | WGHYDEAMDD |
| | (SEQ ID NO: 11) | (SEQ ID NO: 44) | (SEQ ID NO: 87) |
| TM-11 | DYGVH | VIWNNGNTDYNAAFIS | SLRPLHFDY |
| | (SEQ ID NO: 12) | (SEQ ID NO: 45) | (SEQ ID NO: 88) |
| TM-12 | DYYMY | RIDPEDGDAEYAPKFQG | RVIYDGYYRTMDC |
| | (SEQ ID NO: 13) | (SEQ ID NO: 46) | (SEQ ID NO: 89) |
| TM-13 | TFPIE | NFHPYNDDTKYNEKFKG | YFYGGMDY |
| | (SEQ ID NO: 14) | (SEQ ID NO: 47) | (SEQ ID NO: 90) |
| TM-14 | NYLIE | VINPGGGNTDYSEKFKD | SPYSSYVGYAVDY |
| | (SEQ ID NO: 2) | (SEQ ID NO: 48) | (SEQ ID NO: 91) |
| TM-15 | NYWIT | DIYPGSGNSNYNESFKR | KAYGGFPY |
| | (SEQ ID NO: 15) | (SEQ ID NO: 49) | (SEQ ID NO: 92) |
| TM-16 | DYYMH | RIDPEDGETKYAPEFQG | SQPFTY |
| | (SEQ ID NO: 16) | (SEQ ID NO: 50) | (SEQ ID NO: 93) |
| TM-17 | DYLIE | VINPGSGGTNYNEKFKG | SSYGVYVAYPMDY |
| | (SEQ ID NO: 17) | (SEQ ID NO: 51) | (SEQ ID NO: 94) |
| TM-18 | DYYMN | NINPNNGDAFYNQKFKG | EGQLRLRRVYAMDY |
| | (SEQ ID NO: 5) | (SEQ ID NO: 52) | (SEQ ID NO: 95) |
| TM-19 | DYYMY | RIDPEDGDTENAPKFRG | RIGNLYHVMDY |
| | (SEQ ID NO: 13) | (SEQ ID NO: 53) | (SEQ ID NO: 96) |
| TM-20 | DYYMH | RIDPEDGETKYAPEFQG | SQPFTY |
| | (SEQ ID NO: 16) | (SEQ ID NO: 50) | (SEQ ID NO: 93) |
| TM-21 | NYAMS | FISDGGGYIYYADNVKD | DGGTGFTY |
| | (SEQ ID NO: 18) | (SEQ ID NO: 54) | (SEQ ID NO: 97) |
| TM-22 | SYYIH | WIYPGNGITNYNEKFKG | PYYGIRNCYFDV |
| | (SEQ ID NO: 19) | (SEQ ID NO: 55) | (SEQ ID NO: 98) |
| TM-23 | NYLIE | VINPGSGITNYNEKFKG | SDFITTVVADY |
| | (SEQ ID NO: 2) | (SEQ ID NO: 56) | (SEQ ID NO: 99) |
| TM-24 | DYGVH | VIWNNGNTDYNAAFIS | SLRPLHFDY |
| | (SEQ ID NO: 12) | (SEQ ID NO: 45) | (SEQ ID NO: 88) |
| TM-25 | GYGMS | TISSGGRYTVYPDSVKG | DNFYSYAMDY |
| | (SEQ ID NO: 10) | (SEQ ID NO: 57) | (SEQ ID NO: 100) |
| TM-26 | NYLIE | VINPGSGITNYNEKFKG | SDFITTVVADY |
| | (SEQ ID NO: 2) | (SEQ ID NO: 56) | (SEQ ID NO: 99) |
| TM-27 | NYLIE | VINPGSGSTKYNEKFKG | IIYDHDWYFDV |
| | (SEQ ID NO: 2) | (SEQ ID NO: 58) | (SEQ ID NO: 101) |
| TM-28 | DYYIH | LVYPYNGGTNYNQNFKG | SYFSNPIGY |
| | (SEQ ID NO: 8) | (SEQ ID NO: 59) | (SEQ ID NO: 102) |
| TM-29 | DYYIN | RIYPGSGYTYYNEKFKG | HYTNPFAY |
| | (SEQ ID NO: 20) | (SEQ ID NO: 60) | (SEQ ID NO: 103) |
| TM-30 | NYDVN | WIYPRDGTTIYNEKFKG | TLPQAMDY |
| | (SEQ ID NO: 21) | (SEQ ID NO: 61) | (SEQ ID NO: 104) |
| TM-31 | SYWMN | YINPTSGYTRYNQKFKD | SPPTVVLIGYFDY |
| | (SEQ ID NO: 11) | (SEQ ID NO: 62) | (SEQ ID NO: 105) |
| TM-32 | DYYMY | RIDPEDGDTEYVPKFQG | RTWDLYYAVDN |
| | (SEQ ID NO: 13) | (SEQ ID NO: 63) | (SEQ ID NO: 106) |
| TM-33 | DYNMN | VINPNYGTTSYNQKFKG | SY |
| | (SEQ ID NO: 22) | (SEQ ID NO: 64) | (SEQ ID NO: 107) |
| TM-34 | DSGMD | YISSGSSTTHYADTVKG | RDGNYWYFDV |
| | (SEQ ID NO: 23) | (SEQ ID NO: 65) | (SEQ ID NO: 108) |
| TM-35 | RHWMQ | EILPGSNNIYYNEKVKG | SLYDYDGVFAY |
| | (SEQ ID NO: 24) | (SEQ ID NO: 66) | (SEQ ID NO: 109) |
| TM-37 | SYWMH | YVNPSSGYTKNNQKFKD | EGGSISDWYFDV |
| | (SEQ ID NO: 25) | (SEQ ID NO: 67) | (SEQ ID NO: 110) |
| TM-39 | DYWMH | FINPSSGYTKYNQNFKD | EAGSISDWYFDV |
| | (SEQ ID NO: 26) | (SEQ ID NO: 68) | (SEQ ID NO: 111) |
| TM-41 | DCYMH | RIDPEDGTTNFAPKFQD | EWDSGAY |
| | (SEQ ID NO: 27) | (SEQ ID NO: 69) | (SEQ ID NO: 112) |
| TM-42 | NYWMH | YINPSSGYTKYNQKFKD | EGGSISDWYFDV |
| | (SEQ ID NO: 28) | (SEQ ID NO: 70) | (SEQ ID NO: 110) |
| TM-44 | DYGMH | YISSGSSTIYYADTVKG | NYGSPYAMDY |
| | (SEQ ID NO: 29) | (SEQ ID NO: 71) | (SEQ ID NO: 113) |
| TM-45 | NYWMH | YINPSSGYTKYNQKFKD | EGGSISDWYFDV |
| | (SEQ ID NO: 28) | (SEQ ID NO: 70) | (SEQ ID NO: 110) |
| TM-46 | SFWMN | QIYPGDGDTDYNGKFKD | GDGFSYFDY |
| | (SEQ ID NO: 30) | (SEQ ID NO: 72) | (SEQ ID NO: 114) |
| TM-47 | NYGVH | VIWAGGNTNYNSALMS | EAKLLRSYAMDY |
| | (SEQ ID NO: 31) | (SEQ ID NO: 73) | (SEQ ID NO: 115) |
| TM-48 | SYDIN | WIYPRDGNTQYIEKLKG | WIFYAMDY |
| | (SEQ ID NO: 32) | (SEQ ID NO: 74) | (SEQ ID NO: 116) |
| TM-49 | RFWMH | NIDPSDSQTHYNQKFKD | LITVDYAMDY |
| | (SEQ ID NO: 33) | (SEQ ID NO: 75) | (SEQ ID NO: 117) |
| TM-50 | NYGVH | VIWAGGNTNYNSALMS | EAKLLRSYAMDY |
| | (SEQ ID NO: 31) | (SEQ ID NO: 73) | (SEQ ID NO: 115) |
| TM-51 | TYWID | NMFPGSSRTNYNEKFKS | KEGLWTYGYDGGAWFAY |
| | (SEQ ID NO: 34) | (SEQ ID NO: 76) | (SEQ ID NO: 118) |
| TM-52 | NYWMH | NIDPSDSETHYNQKFKD | RGYYGRSPFAY |
| | (SEQ ID NO: 28) | (SEQ ID NO: 77) | (SEQ ID NO: 119) |

TABLE 2

| Antibody | CDR-L1 | CDR-L2 | CDR-L3 |
|---|---|---|---|
| TM-1 | RSSQSIVYNNGNTYLE | KVSNRFS | FQVSHVPFT |
| | (SEQ ID NO: 120) | (SEQ ID NO: 160) | (SEQ ID NO: 184) |
| TM-2 | RASSSVSYIH | ATSNLAS | QQWSSNPST |
| | (SEQ ID NO: 121) | (SEQ ID NO: 161) | (SEQ ID NO: 185) |
| TM-3 | RSSKSLLHSNGITYLY | QMSSLAS | AQNLELPWT |
| | (SEQ ID NO: 122) | (SEQ ID NO: 162) | (SEQ ID NO: 186) |
| TM-4 | RSSTGAVTTSNYAN | GTNNRAP | VLWYSNHLV |
| | (SEQ ID NO: 123) | (SEQ ID NO: 163) | (SEQ ID NO: 187) |
| TM-5 | RASENIYIYLA | NGKMLAE | QHHYGSPPA |
| | (SEQ ID NO: 124) | (SEQ ID NO: 164) | (SEQ ID NO: 188) |
| TM-6 | KASDHINNWLA | GATSLET | QQYWSSPYT |
| | (SEQ ID NO: 125) | (SEQ ID NO: 165) | (SEQ ID NO: 189) |
| TM-7 | KSSQSLLNSGNQRNYLA | GASTRES | QNDHSYPLT |
| | (SEQ ID NO: 126) | (SEQ ID NO: 166) | (SEQ ID NO: 190) |
| TM-8 | KASQDINSYLS | RANRLVD | LQYDEFPLT |
| | (SEQ ID NO: 127) | (SEQ ID NO: 167) | (SEQ ID NO: 191) |
| TM-9 | RSSKSLLHSNGITYLY | QMSNLAS | AQNLELPWT |
| | (SEQ ID NO: 122) | (SEQ ID NO: 168) | (SEQ ID NO: 186) |
| TM-10 | RSSQSLVHSNGKTYLH | KISNRFS | SQITHVPWT |
| | (SEQ ID NO: 128) | (SEQ ID NO: 169) | (SEQ ID NO: 192) |
| TM-11 | KSSQSLLNSNNLQNYLA | FASIRES | QQHYNTPFT |
| | (SEQ ID NO: 129) | (SEQ ID NO: 170) | (SEQ ID NO: 193) |
| TM-12 | RSSQTIVHSNGNTYLE | KISNRFS | FQGSHVPYT |
| | (SEQ ID NO: 130) | (SEQ ID NO: 169) | (SEQ ID NO: 194) |
| TM-13 | RASENIYSSLG | AATNLAD | QHLWSIPWT |
| | (SEQ ID NO: 131) | (SEQ ID NO: 171) | (SEQ ID NO: 195) |
| TM-14 | RASQDIGSNLN | ATSSLDS | LQYASSPRT |
| | (SEQ ID NO: 132) | (SEQ ID NO: 172) | (SEQ ID NO: 196) |
| TM-15 | RVSENIYNNLA | AATNLAD | QHFWDTPFT |
| | (SEQ ID NO: 133) | (SEQ ID NO: 171) | (SEQ ID NO: 197) |
| TM-16 | SASSSLNYMY | DTSNLAS | QQWTSFPPT |
| | (SEQ ID NO: 134) | (SEQ ID NO: 173) | (SEQ ID NO: 198) |
| TM-17 | RASQDIGSNLN | ATSSLDS | LQYASSPRT |
| | (SEQ ID NO: 132) | (SEQ ID NO: 172) | (SEQ ID NO: 196) |
| TM-18 | RASKSVSISVYTYVH | LASNLES | QHSRELPYT |
| | (SEQ ID NO: 135) | (SEQ ID NO: 174) | (SEQ ID NO: 199) |
| TM-19 | RSSQSIVHSNGNTYLE | KVFNRFS | FQGSHVPFT |
| | (SEQ ID NO: 136) | (SEQ ID NO: 175) | (SEQ ID NO: 200) |
| TM-20 | SASSSLNYMY | DTSNLAS | QQWTSFPPT |
| | (SEQ ID NO: 134) | (SEQ ID NO: 173) | (SEQ ID NO: 198) |
| TM-21 | RSSKSLLHSNGITYLF | QMSNLAS | VQNLELPYT |
| | (SEQ ID NO: 137) | (SEQ ID NO: 168) | (SEQ ID NO: 201) |
| TM-22 | RSGQSIVHSNGNTYLE | KVSNRFS | FQGSHVPWT |
| | (SEQ ID NO: 138) | (SEQ ID NO: 160) | (SEQ ID NO: 202) |
| TM-23 | RSSQSLTNYYGNTYLS | GISNRFS | LQGTHQPRT |
| | (SEQ ID NO: 139) | (SEQ ID NO: 176) | (SEQ ID NO: 203) |
| TM-24 | KSSQSLLNSNNQQNYLA | FASIRES | QQHYSTPFT |
| | (SEQ ID NO: 140) | (SEQ ID NO: 170) | (SEQ ID NO: 204) |
| TM-25 | RSSKSLLHSNGITYLF | QMSNLAS | AQNLELWT |
| | (SEQ ID NO: 137) | (SEQ ID NO: 168) | (SEQ ID NO: 205) |
| TM-26 | RSSQSLTNYYGNTYLS | GISNRFS | LQGTHQPRT |
| | (SEQ ID NO: 139) | (SEQ ID NO: 176) | (SEQ ID NO: 203) |
| TM-27 | SASSSISYMY | RTSTLAS | QQYHSYPRT |
| | (SEQ ID NO: 141) | (SEQ ID NO: 177) | (SEQ ID NO: 206) |
| TM-28 | KSSQSLLNSGNQKNYLA | GASTRES | QNDHSYPLT |
| | (SEQ ID NO: 142) | (SEQ ID NO: 166) | (SEQ ID NO: 190) |
| TM-29 | RSSKSLLHYNGITYLY | QMSNLAS | AQNLELPYT |
| | (SEQ ID NO: 143) | (SEQ ID NO: 168) | (SEQ ID NO: 207) |
| TM-30 | RSSQTIVHRNGNTYLE | KVSNRFS | FQGSHLPWT |
| | (SEQ ID NO: 144) | (SEQ ID NO: 160) | (SEQ ID NO: 208) |
| TM-31 | KASQDINSYLS | RGNGLVD | LQYDEFPFT |
| | (SEQ ID NO: 127) | (SEQ ID NO: 178) | (SEQ ID NO: 209) |
| TM-32 | RSSQNIVHSNGNTYLE | KVSNRFS | FQGSHVPFT |
| | (SEQ ID NO: 145) | (SEQ ID NO: 160) | (SEQ ID NO: 200) |
| TM-33 | KASQNVGTAVA | SASNRYT | QQYSSYPYT |
| | (SEQ ID NO: 146) | (SEQ ID NO: 179) | (SEQ ID NO: 210) |
| TM-34 | RATSSVTYMH | ATSNLAS | QQWSSNPYT |
| | (SEQ ID NO: 147) | (SEQ ID NO: 161) | (SEQ ID NO: 211) |
| TM-35 | RSSQSIVHRNGNTYLE | KVSNRFS | FQGSHVPYT |
| | (SEQ ID NO: 148) | (SEQ ID NO: 160) | (SEQ ID NO: 194) |
| TM-37 | RSSKSLLHSNGNTYSY | RMSNLAS | MQHLEYPYT |
| | (SEQ ID NO: 149) | (SEQ ID NO: 180) | (SEQ ID NO: 212) |
| TM-39 | RSSKTLLNSNGNTYLY | RMSNLAS | MQHLDYPYT |
| | (SEQ ID NO: 150) | (SEQ ID NO: 180) | (SEQ ID NO: 213) |
| TM-41 | KSSQSLLYSSNQKNYLA | WASTRES | QQYYSYPYT |
| | (SEQ ID NO: 151) | (SEQ ID NO: 181) | (SEQ ID NO: 214) |
| TM-42 | RSSKSLLHSNGNTYLY | RMSNLAS | MQHLEYPYT |
| | (SEQ ID NO: 152) | (SEQ ID NO: 180) | (SEQ ID NO: 212) |
| TM-44 | KSSQSLLYSSNQKNYLA | WASTRES | QQYYSYPT |
| | (SEQ ID NO: 151) | (SEQ ID NO: 181) | (SEQ ID NO: 215) |
| TM-45 | RSSKSLLHSNGNTYLY | RMSNLAS | MQHLEYPYT |
| | (SEQ ID NO: 152) | (SEQ ID NO: 180) | (SEQ ID NO: 212) |
| TM-46 | CASSRVNYMH | DTSKLAS | QQWSSNPPT |
| | (SEQ ID NO: 153) | (SEQ ID NO: 182) | (SEQ ID NO: 216) |
| TM-47 | KSSQSLLNSGNQKNYLT | WASTRES | QNDYSYPLT |
| | (SEQ ID NO: 154) | (SEQ ID NO: 181) | (SEQ ID NO: 217) |
| TM-48 | RSSQSIVHGNGNTYLE | KVSNRFS | FQGSHLPYT |
| | (SEQ ID NO: 155) | (SEQ ID NO: 160) | (SEQ ID NO: 218) |
| TM-49 | SASSSVSYMY | RTSNLAS | QQYHSYPPT |
| | (SEQ ID NO: 156) | (SEQ ID NO: 182) | (SEQ ID NO: 219) |
| TM-50 | RASQDINNYLY | YTSMLHS | QQGSTLMYT |
| | (SEQ ID NO: 157) | (SEQ ID NO: 183) | (SEQ ID NO: 220) |
| TM-51 | KSSQSLRNSRTRKNYLA | WASTRES | KQSYNLLT |
| | (SEQ ID NO: 158) | (SEQ ID NO: 181) | (SEQ ID NO: 221) |
| TM-52 | SASSSVSYMH | DTSKLAS | FQGSGYPLT |
| | (SEQ ID NO: 159) | (SEQ ID NO: 182) | (SEQ ID NO: 222) |

TABLE 3

| Antibody | CDR-H1 | CDR-H2 | CDR-H3 |
|---|---|---|---|
| TM-54 | TYDLN | WIYPRGGSTEYNEKFKG | YSIYAMDY |
| | (SEQ ID NO: 223) | (SEQ ID NO: 244) | (SEQ ID NO: 277) |
| TM-56 | SYWMH | YINPSSGYSKYNQRFKD | EGGSISDWYFDV |
| | (SEQ ID NO: 25) | (SEQ ID NO: 245) | (SEQ ID NO: 110) |
| TM-59 | SYWMH | YVNPSSGYTKNNQKFKD | EGGSISDWYFDV |
| | (SEQ ID NO: 25) | (SEQ ID NO: 67) | (SEQ ID NO: 110) |
| TM-60 | SYDIN | WIYPRDGNTQYIEKLKG | WIFYAMDY |
| | (SEQ ID NO: 32) | (SEQ ID NO: 74) | (SEQ ID NO: 116) |
| TM-61 | NYWMN | YINPSSDYTKYNQNFKD | WGGSRSYWYFDV |
| | (SEQ ID NO: 224) | (SEQ ID NO: 246) | (SEQ ID NO: 278) |
| TM-62 | RYWMH | NIDPSDSETQYNPKFKD | LTTLDYAMDY |
| | (SEQ ID NO: 225) | (SEQ ID NO: 247) | (SEQ ID NO: 279) |
| TM-63 | SFWMH | NIDPSDSETHYSQKFKD | SSNWDNFDY |
| | (SEQ ID NO: 226) | (SEQ ID NO: 248) | (SEQ ID NO: 280) |
| TM-64 | SYWMH | NIDPSDSDTHYNQNFRG | SRGGTYDYGEAMDY |
| | (SEQ ID NO: 25) | (SEQ ID NO: 249) | (SEQ ID NO: 281) |
| TM-65 | SSWMH | EINPKNGGTNYNEKFKT | PTWFTY |
| | (SEQ ID NO: 227) | (SEQ ID NO: 250) | (SEQ ID NO: 282) |
| TM-66 | NYAMS | TIGSVGSYTYYPDSVKG | QEGSSHFDY |
| | (SEQ ID NO: 18) | (SEQ ID NO: 251) | (SEQ ID NO: 283) |
| TM-68 | DDYMH | WIDPENGDTEYASKFQG | PQTATTC |
| | (SEQ ID NO: 228) | (SEQ ID NO: 252) | (SEQ ID NO: 284) |
| TM-69 | SYGVH | IMGWDDKKYYNSALKS | EAGPYGMDY |
| | (SEQ ID NO: 229) | (SEQ ID NO: 253) | (SEQ ID NO: 285) |
| TM-70 | NYAMS | TIGYGGSYSYYPDSVKG | QDSNYEGVWFAY |
| | (SEQ ID NO: 18) | (SEQ ID NO: 254) | (SEQ ID NO: 286) |
| TM-71 | NYAMS | TIGYGGSYSYYPDSVKG | QDSNYEGVWFAY |
| | (SEQ ID NO: 18) | (SEQ ID NO: 254) | (SEQ ID NO: 286) |
| TM-72 | NYWMH | NIDPSDSDTHYNQKFKD | SRGGYYDYGEAMDY |
| | (SEQ ID NO: 28) | (SEQ ID NO: 255) | (SEQ ID NO: 287) |
| TM-73 | SHWMH | EIDPSDSYTYYNQKFKG | NYHGSGPPFAH |
| | (SEQ ID NO: 230) | (SEQ ID NO: 256) | (SEQ ID NO: 288) |
| TM-74 | SYVIH | FINPNNDGTKYNEKFRG | PYYDYEGYTMDY |
| | (SEQ ID NO: 231) | (SEQ ID NO: 257) | (SEQ ID NO: 289) |
| TM-75 | SYVMH | YINPYNDGTKYNEKFKG | GGWFPYAMDY |
| | (SEQ ID NO: 232) | (SEQ ID NO: 258) | (SEQ ID NO: 290) |
| TM-76 | TSGMGVS | HIFWDDDKRYNLFLKS | MGRQRDYFDY |
| | (SEQ ID NO: 233) | (SEQ ID NO: 259) | (SEQ ID NO: 291) |
| TM-77 | DYEMH | VIDPETGGTAYNQMFKG | YGIEGY |
| | (SEQ ID NO: 234) | (SEQ ID NO: 260) | (SEQ ID NO: 292) |
| TM-78 | NHWMH | NIDPSDSDTHYNHKFKD | SRGGYYDYGEAMDY |
| | (SEQ ID NO: 235) | (SEQ ID NO: 261) | (SEQ ID NO: 287) |
| TM-79 | NYVMH | HITPHNDGTKYNEKFKG | SGQFGRGDYFHY |
| | (SEQ ID NO: 236) | (SEQ ID NO: 262) | (SEQ ID NO: 293) |
| TM-80 | TFWMH | NIDPSDSETHYNQEFKD | SSNWDNFDY |
| | (SEQ ID NO: 237) | (SEQ ID NO: 263) | (SEQ ID NO: 280) |
| TM-81 | SYWMH | NIDPSDSEIHYNQKFKD | SPAGQILDY |
| | (SEQ ID NO: 25) | (SEQ ID NO: 264) | (SEQ ID NO: 294) |
| TM-82 | NYLIE | VINPGSGITDYSEKFKG | SPFITTVVADY |
| | (SEQ ID NO: 2) | (SEQ ID NO: 265) | (SEQ ID NO: 295) |
| TM-83 | TYWIH | NINPSNGNTKNNERFKT | GDYYGTSYPF |
| | (SEQ ID NO: 238) | (SEQ ID NO: 266) | (SEQ ID NO: 296) |
| TM-84 | TFPIE | NFHPYNDDTKYNEKFKG | YFYGGMDY |
| | (SEQ ID NO: 14) | (SEQ ID NO: 47) | (SEQ ID NO: 90) |
| TM-85 | GYWMH | RIDPYSGDTRYNEKFKN | YGLGHAMDY |
| | (SEQ ID NO: 239) | (SEQ ID NO: 267) | (SEQ ID NO: 297) |
| TM-86 | DYYIH | VISPYNDNTNYNQKFKG | TYYSNYFDY |
| | (SEQ ID NO: 8) | (SEQ ID NO: 268) | (SEQ ID NO: 298) |
| TM-87 | DYYMN | VINPYNGATSYNQKFQD | GNYDWYFDV |
| | (SEQ ID NO: 5) | (SEQ ID NO: 269) | (SEQ ID NO: 299) |
| TM-88 | DYYIN | VINPYSGATYYTQKFKG | GGYDYGHWYFDV |
| | (SEQ ID NO: 20) | (SEQ ID NO: 270) | (SEQ ID NO: 300) |
| TM-89 | NYLIE | VINPGSGGTDYNEKFKG | DPYSKYVHYPMDY |
| | (SEQ ID NO: 2) | (SEQ ID NO: 271) | (SEQ ID NO: 301) |
| TM-90 | SYDIN | WIYPRDGNAKYNEKFKG | VSDYHGGYGMDY |
| | (SEQ ID NO: 32) | (SEQ ID NO: 272) | (SEQ ID NO: 302) |
| TM-91 | SGYYWD | YISYDGNNNYNPSLKN | AVYSVGFAY |
| | (SEQ ID NO: 240) | (SEQ ID NO: 273) | (SEQ ID NO: 303) |
| TM-92 | DYTMD | HINPNDGGTFYNQKFKG | WVYYYGISYEGGAMDY |
| | (SEQ ID NO: 241) | (SEQ ID NO: 274) | (SEQ ID NO: 304) |
| TM-93 | SYGIS | VIWTGGGTNYNSALES | NPPKIFYDYLMYFDY |
| | (SEQ ID NO: 242) | (SEQ ID NO: 275) | (SEQ ID NO: 305) |
| TM-94 | DYNMD | DINPYNGGTLYNQKFKG | VGYHGTPDY |
| | (SEQ ID NO: 243) | (SEQ ID NO: 276) | (SEQ ID NO: 306) |

TABLE 4

| Antibody | CDR-L1 | CDR-L2 | CDR-L3 |
|---|---|---|---|
| TM-54 | RSSQSIVHGNGNTYLE | KVSNRFS | FQGSHVPWT |
| | (SEQ ID NO: 155) | (SEQ ID NO: 160) | (SEQ ID NO: 202) |
| TM-56 | RSSKSLLQNNGNTYLY | RMSNLAS | MQHLEYPYT |
| | (SEQ ID NO: 307) | (SEQ ID NO: 180) | (SEQ ID NO: 212) |
| TM-59 | RSSKSLLHSNGNTYSY | RMSNLAS | MQHLEYPYT |
| | (SEQ ID NO: 149) | (SEQ ID NO: 180) | (SEQ ID NO: 212) |
| TM-60 | RSSQSIVHGNGNTYLE | KVSNRFS | FQGSHLPYT |
| | (SEQ ID NO: 155) | (SEQ ID NO: 160) | (SEQ ID NO: 218) |
| TM-61 | KSSQSLLNSGNQKNYLA | GASTRES | QNDSNYPFT |
| | (SEQ ID NO: 142) | (SEQ ID NO: 166) | (SEQ ID NO: 349) |
| TM-62 | SASSSVSYMY | RTSNLAS | QQYHSYPPT |
| | (SEQ ID NO: 156) | (SEQ ID NO: 182) | (SEQ ID NO: 219) |
| TM-63 | RSSQSLLHVNGNTYLY | RMSNLAS | MQHLQYPFS |
| | (SEQ ID NO: 308) | (SEQ ID NO: 180) | (SEQ ID NO: 350) |
| TM-64 | RSSKSLLHTNGNTYLF | RMSNLAS | MQHLEYPFT |
| | (SEQ ID NO: 309) | (SEQ ID NO: 180) | (SEQ ID NO: 351) |
| TM-65 | RASQNINIWLS | KASNLHT | LQGQSYPYT |
| | (SEQ ID NO: 310) | (SEQ ID NO: 335) | (SEQ ID NO: 352) |
| TM-66 | RSSQSLVNSYGKTFLS | GISNRFS | LQGTHPPLT |
| | (SEQ ID NO: 311) | (SEQ ID NO: 176) | (SEQ ID NO: 353) |
| TM-68 | SSSKSLLHSSGITYLY | RMSNLAS | AQMLERPWT |
| | (SEQ ID NO: 312) | (SEQ ID NO: 180) | (SEQ ID NO: 354) |
| TM-69 | RSSQSLVHSNGNTYLH | KVSNRFS | SQNTHVPLT |
| | (SEQ ID NO: 313) | (SEQ ID NO: 160) | (SEQ ID NO: 355) |
| TM-70 | RASESVDNYGISFMN | AASNQGS | QQSKDVPWT |
| | (SEQ ID NO: 314) | (SEQ ID NO: 336) | (SEQ ID NO: 356) |
| TM-71 | RSSKSLLHTNGNTYLF | RKSNLAS | MQHLEYPFT |
| | (SEQ ID NO: 309) | (SEQ ID NO: 337) | (SEQ ID NO: 351) |
| TM-72 | RSSKSLLHTNGNTYLF | RKSNLAS | MQHLEYPFT |
| | (SEQ ID NO: 309) | (SEQ ID NO: 337) | (SEQ ID NO: 351) |
| TM-73 | SASSGISYIY | DTSNLAS | HQWRSYPPT |
| | (SEQ ID NO: 315) | (SEQ ID NO: 173) | (SEQ ID NO: 357) |
| TM-74 | KASQNVRSAVA | LASNRHT | LQHWKYPFT |
| | (SEQ ID NO: 316) | (SEQ ID NO: 338) | (SEQ ID NO: 358) |
| TM-75 | RASQSVDYNGISYMH | AASNLES | QQSIEDPWT |
| | (SEQ ID NO: 317) | (SEQ ID NO: 339) | (SEQ ID NO: 359) |
| TM-76 | SASLSISYVH | GTSNLAS | HQWSIYRT |
| | (SEQ ID NO: 318) | (SEQ ID NO: 340) | (SEQ ID NO: 360) |
| TM-77 | KSSQSLFHSNGKTYLN | MVSKLES | LQVTHFPLT |
| | (SEQ ID NO: 319) | (SEQ ID NO: 341) | (SEQ ID NO: 361) |
| TM-78 | RSSKSLLHVNGNTYLF | RMSNLAS | MQHLEYPFT |
| | (SEQ ID NO: 320) | (SEQ ID NO: 180) | (SEQ ID NO: 351) |
| TM-79 | RASKSVSTSGSIYIH | LTSNLES | QHSGELPWT |
| | (SEQ ID NO: 321) | (SEQ ID NO: 342) | (SEQ ID NO: 362) |
| TM-80 | RSSQSLLHVNGHTYLY | RMSNLAS | MQHLQYPFT |
| | (SEQ ID NO: 322) | (SEQ ID NO: 180) | (SEQ ID NO: 363) |
| TM-81 | KSSQSLLNSKNQKNYLA | FAFFRES | QQHYSTPFT |
| | (SEQ ID NO: 323) | (SEQ ID NO: 343) | (SEQ ID NO: 204) |
| TM-82 | RSSQSLANTYGNTYLS | GISNRFS | LQGTHPPRT |
| | (SEQ ID NO: 324) | (SEQ ID NO: 176) | (SEQ ID NO: 364) |
| TM-83 | RSSQSLVHSNGITYLH | KVSNRFS | SQSTHVPYT |
| | (SEQ ID NO: 325) | (SEQ ID NO: 160) | (SEQ ID NO: 365) |
| TM-84 | RASENIYSSLG | AATNLAD | QHLWSVPWT |
| | (SEQ ID NO: 131) | (SEQ ID NO: 171) | (SEQ ID NO: 366) |
| TM-85 | KASQNVGTNVA | SASYRYS | QQYNSYPLT |
| | (SEQ ID NO: 326) | (SEQ ID NO: 344) | (SEQ ID NO: 367) |
| TM-86 | KSSQSLLNSGNQKNYLA | GASTRES | QNDHSYPLT |
| | (SEQ ID NO: 142) | (SEQ ID NO: 166) | (SEQ ID NO: 190) |
| TM-87 | RASRSISKYLA | SGSTLQS | QQHNEYPLT |
| | (SEQ ID NO: 327) | (SEQ ID NO: 345) | (SEQ ID NO: 368) |
| TM-88 | ITSSDIDDHMN | EGNALRP | LQSDNLPLT |
| | (SEQ ID NO: 328) | (SEQ ID NO: 346) | (SEQ ID NO: 369) |
| TM-89 | RASQDIGSSLN | ATSSLDS | LQYASSPRT |
| | (SEQ ID NO: 329) | (SEQ ID NO: 172) | (SEQ ID NO: 196) |
| TM-90 | RSSQSFVHGNGNTYLE | KVSHRFS | FQNSHVPHT |
| | (SEQ ID NO: 330) | (SEQ ID NO: 347) | (SEQ ID NO: 370) |
| TM-91 | KASQNVYTNVA | SASYRYS | QQYNSYPLT |
| | (SEQ ID NO: 331) | (SEQ ID NO: 344) | (SEQ ID NO: 367) |
| TM-92 | RASKSISKYLA | SGSTLQS | QQHNEYPWT |
| | (SEQ ID NO: 332) | (SEQ ID NO: 345) | (SEQ ID NO: 371) |
| TM-93 | KASQDVGSAVA | WSSTRLP | QQYTSYPLT |
| | (SEQ ID NO: 333) | (SEQ ID NO: 348) | (SEQ ID NO: 372) |
| TM-94 | RSSQSLVYSNGNTYLH | KVSNRFS | SQSTHVYT |
| | (SEQ ID NO: 334) | (SEQ ID NO: 160) | (SEQ ID NO: 373) |

TABLE 5

| Antibody | Variable Heavy Chain | Variable Light Chain |
|---|---|---|
| TM-1 | QVQLQQSGAELVRPGTSVKVSCKASGYA | DVLMTQTPLSLPVSLGDHASISCRSS |
| | FTNYLIEWVKQRPGQGLEWIGVINPGSGG | QSIVYNNGNTYLEWYLQKPGQSPKL |
| | TKYNEKLKGKATLTADKSSSTAYMQLSS | LIYKVSNRFSGVPDRFSGSGSGTDFTL |
| | LTSVDSAVYFCARRGYTIYDFYAMDYWG | KISRVEAEDLGVYYCFQVSHVPFTFG |
| | QGTSVTVSS (SEQ ID NO: 374) | SGTKLEIK (SEQ ID NO: 418) |
| TM-2 | HIQLVQSGPELKKPGETVKISCKASGYTFT | QIVLTQSPAILSASPGEKVTMTCRASS |
| | EYPMHWVKQAPGKGFRWMGMIYTNTGE | SVSYIHWFLQKPGSSPKPWIYATSNL |
| | PTYAAEFKGRFAFSLETSASTGYLQINNLK | ASGVPFRFIGSGSGTSYSLTISGVEAE |
| | NEDSATYFCVTAGYWGQGTLVTVSA | DSATYYCQQWSSNPSTFGAGTKLEL |
| | (SEQ ID NO: 375) | K (SEQ ID NO: 419) |
| TM-3 | QVTLKESGPGIVQPSQTLSLTCSFSGFSLN | DIVMTQAAFSNPVTLGTSASISCRSSK |
| | TLGRGVGWIRQPSGKGLEWLAKIWWND | SLLHSNGITYLYWYLQKPGQSPQLLI |
| | DKFYYPALKSRLTISKDTSKNQIFLKIANV | SQMSSLASGVPDRESSGGSGTDFTLRI |
| | DTADSATYYCARIAGGTGAAYWGQGTTL | SRVEAEDVGVFYCAQNLELPWTFGG |
| | TVSS (SEQ ID NO: 376) | GTKLELK (SEQ ID NO: 420) |
| TM-4 | EVQVQQSGPVLVKPGASVKMSCKASGYT | QAVVTQESALTTSPGETVTLTCRSST |
| | FTDYYMNWVKQSHGKNLEWIGVINPYN | GAVTTSNYANWVQEKPDHLFTGLIG |
| | GGTSYHQKFKGKATLTVDKSSSTAYMEL | GTNNRAPGVPARFSGSLIGDKAALTI |
| | NSLTSEDSAVYYCARAATVVAGFDYWG | TGAQTEDEAIYFCVLWYSNHLVFGG |
| | QGTTLTVSS (SEQ ID NO: 377) | GTKLTVL (SEQ ID NO: 421) |
| TM-5 | QVQLQQSGTELARPGASVKVSCKASGYIF | DIQMTQSPASLSASVGETVTITCRASE |
| | TTYGITWVKQRGGQGLEWIGEIYPRSDNT | NIYIYLAWYQQKQGKTPQLLVYNGK |
| | YYNEKFKDKATLTADKSSSTAYMELRSL | MLAENVPSRFSGSGSGTQFSLKINSL |
| | TSEDSAVYFCARSKGSGTGDYWGQGTTV | QPEDFGSYYCQHHYGSPPAFGAGTK |
| | TVSS (SEQ ID NO: 378) | LELK (SEQ ID NO: 422) |
| TM-6 | QVQLQQSGAELVKPGASVKLSCKASGYT | DIQMSQSSSYLSVSLGGRVTITCKAS |
| | FTEYTIHWVKQRSGQGLEWIGWFYPGST | DHINNWLAWYQQKPGNAPRLLISGA |
| | YIDYNEKFKDKATLTADKSSSTVYLELSR | TSLETGVPSRFSGSGSGKDYTLSITSL |
| | LTSEDSAVYFCARHEEDYSNWFPFWGQG | QTEDVATYYCQQYWSSPYTFGGGTK |
| | TLVTVSA (SEQ ID NO: 379) | LEIK (SEQ ID NO: 423) |
| TM-7 | EVQLQQSGPVLVKPGPPVKISCKASGFTF | DIVMTQSPSSLSVSAGEKVTMSCKSS |
| | TDYYIHWVKLSHGKSLEWIGLVYPYNGD | QSLLNSGNQRNYLAWYQQKPGQPPK |
| | TDYNQKFKGKATLTVDTSSSTAYMELNS | LLIYGASTRESGVPDRFTGSGSGTDFT |
| | LTSEDSAVYYCARTYYANSPDYWGQGTT | LTISNVQAEDLAVYYCQNDHSYPLTF |
| | VTVSS (SEQ ID NO: 380) | GAGTKLELK (SEQ ID NO: 424) |
| TM-8 | QIQLVQSGPELKKPGETVKISCKASGYTFT | DIKMTQSPSSMYASLGERVTITCKAS |
| | DYPMHWVKQAPGKGFKWMGVIYTDTGE | QDINSYLSWFQQKPGKSPKTLIFRAN |
| | PKYAEVFKGRFAFSLETSASTAYLQINNL | RLVDGVPSRFSGSGSGQDYSLTISSLE |
| | KNEDTATYFCVRRLAYWGQGTLVTVSA | YEDMGIYYCLQYDEFPLTFGAGTKL |
| | (SEQ ID NO: 381) | EMK (SEQ ID NO: 425) |
| TM-9 | EVQLVESGGDLVKPGGSLKLSCVASGFTF | DIVMTQAAFSNPVTLGTSASISCRSSK |
| | SGYGMSWVRQTPDKRLEWVATISSGSFYI | SLLHSNGITYLYWYLQKPGQSPQLLI |
| | YYPDSVKGRLTVSRDNAKNTLYLQMSSL | YQMSNLASGVPNRFSSSGSGTDFTLR |
| | KSEDTAIYYCARQNFYYGCEDYWGQGTT | ISRVEAEDVGVYYCAQNLELPWTFG |
| | LTVSS (SEQ ID NO: 382) | GGTKLEIK (SEQ ID NO: 426) |
| TM-10 | QVQLQQSGAELVKPGASVKISCKGSGYA | DVVMTQTPLSLPVSLGDQASISCRSS |
| | FSSYWMNWVKQRPGKGLEWIGQIYPGDG | QSLVHSNGKTYLHWYVQKPGQSPKL |
| | DTNYNGKFKGKATLTADKSSTTAYIHLSS | LIYKISNRFSGVPDRFSGSGSGTDFTL |
| | LTSEDSAVYFCARWGHYDEAMDDWGQG | KISRVEAEDLGVYFCSQITHVPWTFG |
| | TSVTVSS (SEQ ID NO: 383) | GGTKLESK (SEQ ID NO: 427) |
| TM-11 | QVQLKQSGPGQVQPSQSLSITCTVSGFSLS | DIVMTQSPSSLTMSVGQKVTMHCKS |
| | DYGVHWVRQSPGKGLEWLGVIWNNGNT | SQSLLNSNNLQNYLAWYQQKPGQSP |
| | DYNAAFISRLSINKDNSKSQVFFKMTSLQ | TLLVYFASIRESGVPDRFIGSGSGTDF |
| | ADDTAIYYCVRSLRPLHFDYWGQGTTVT | TLTISSVQAEDLADYFCQQHYNTPFT |
| | VSS (SEQ ID NO: 384) | FGSGTKLEIR (SEQ ID NO: 428) |
| TM-12 | EVQLQQSGAELMRPGASVKLSCTASGFNI | DVLMTQIPLSLPVSLGDQASISCRSSQ |
| | QDYYMYWVKQRPEQGLEWIGRIDPEDGD | TIVHSNGNTYLEWYLKKPGQSPKLLI |
| | AEYAPKFQGKATMTADTSSNTAYLQLSS | DKISNRFSGVPDRFSGSGSGTDFTLKI |
| | LTSEDTAVYYCSTRVIYDGYYRTMDCWG | SRVEAEDLGVYYCFQGSHVPYTFGG |
| | QGTSVTVSS (SEQ ID NO: 385) | GTKLEIK (SEQ ID NO: 429) |
| TM-13 | QVQLQQSGAELVKPGASVKMSCKASGYT | DIQMTQSPASLSVSVGETVTITCRASE |
| | FTTFPIEWMKQSHGKGLEWIGNFHPYND | NIYSSLGWYQQKQGESPQLLVFAAT |
| | DTKYNEKFKGKATLTVDKSSSTVYLDLSR | NLADGVPSRFSGSGSGTQYSLKINSL |
| | LTSDDSAVYYCARYFYGGMDYWGQGTS | QSEDFGTYYCQHLWSIPWTFGGGTR |
| | VTVSS (SEQ ID NO: 386) | LEIK (SEQ ID NO: 430) |
| TM-14 | QVQLQQSGPELVRPGTSVKVSCKASGYA | DIQMTQSPSSLSASLGERVSLTCRAS |
| | FTNYLIEWVKQRPGQGLEWIGVINPGGGN | QDIGSNLNWLQQEPDGTIKRLIYATS |
| | TDYSEKFKDKATLTADKSSNTAYIQLSSL | SLDSGVPKRFSGSRSGSDYSLTISSLE |
| | TSEDSAVYFCARSPYSSYVGYAVDYWGQ | SEDFVVYYCLQYASSPRTFGGGTRLE |
| | GTSVTVSS (SEQ ID NO: 387) | IK (SEQ ID NO: 431) |
| TM-15 | QVQLQQPGAEFVRPGASVKLSCKASGYT | DIQMTQSPASLSVSVGETVTITCRVSE |
| | FTNYWITWVKQRPGHGLEWIGDIYPGSG | NIYNNLAWYQQKQEKSPQLLVFAAT |
| | NSNYNESFKRKATLTVDTSSSTAYMHLSS | NLADGVPSRFSGSGSGTQFSLKINSL |
| | QTSEDSAVYFCARKAYGGFPYWGQGTLV | QSEDFGTYYCQHFWDTPFTFGSGTK |
| | TVSA (SEQ ID NO: 388) | LEIK (SEQ ID NO: 432) |
| TM-16 | EVQLQQSGAELVRPGASVKLSCTTSGFNI | QIVLTQSPAIMSASPGEKLTMTCSASS |
| | KDYYMHWVKQRTEQGLEWIGRIDPEDGE | SLNYMYWYQQKPGSSPRLLIYDTSN |
| | TKYAPEFQGKATITSDTSSNTAFLQLSSLT | LASGVPVRFSGSGSGTSYSLTISRME |
| | SEDTAVYYCASSQPFTYWGQGTLVTVSA | AEDGATYYCQQWTSFPPTFGAGTKL |
| | (SEQ ID NO: 389) | ELK (SEQ ID NO: 433) |
| TM-17 | QVQLQQSGAELIRPGTSVKVSCKASGYAF | DIQMTQSPSSLSASLGERVSLTCRAS |
| | TDYLIEWVKQRPGQGLEWIGVINPGSGGT | QDIGSNLNWLQQEPDGTIKRLIYATS |
| | NYNEKFKGKAKLTADKSSSTAYMQLSSL | SLDSGVPKRFSGSRSGSDYSLTISSLE |
| | TSEDSAVYFCVRSSYGVYVAYPMDYWG | SEDFVDYYCLQYASSPRTFGGGTKLE |
| | QGTSVTVSS (SEQ ID NO: 390) | IK (SEQ ID NO: 434) |
| TM-18 | EVRLQQSGPELVKPGASVKISCKTSGYTF | DIVLAQSPASLAVSLGQRATISCRAS |
| | TDYYMNWVKQSHGKSLEWIVNINPNNG | KSVSISVYTYVHWYQQKPGQPPKLLI |
| | DAFYNQKFKGKATLTVDKSSNTAYLDLR | YLASNLESGVPARFSGSGSGTDFTLNI |
| | SLTSEDSAVYYCAREGQLRLRRVYAMDY | HPVEEEDAATYYCQHSRELPYTFGG |
| | WGQGTSVTVSS (SEQ ID NO: 391) | GTKLEIK (SEQ ID NO: 435) |
| TM-19 | DVQLQQSGAELVRPGASVKLSCTASGFNI | DVLMTQTPLSLPVSLGDQASISCRSS |
| | KDYYMYWVKQRPEQGLEWIGRIDPEDGD | QSIVHSNGNTYLEWYLQKPGQAPKL |
| | TENAPKFRGMATMTADTSSNTAYLQLNS | LIDKVFNRFSGVPDRFSGSGSGTDFTL |
| | LTSEDTAVYYCTTRIGNLYHVMDYWGHG | KISRVEAEDLGVYYCFQGSHVPFTFG |
| | TSVTVSS (SEQ ID NO: 392) | SGTKLEIK (SEQ ID NO: 436) |
| TM-20 | EVQLQQSGAELVRPGASVKLSCTTSGFNI | QIVLTQSPAIMSASPGEKLTMTCSASS |
| | KDYYMHWVKQRTEQGLEWIGRIDPEDGE | SLNYMYWYQQKPGSSPRLLIYDTSN |
| | TKYAPEFQGKATITSDTSSNTAFLQLSSLT | LASGVPVRFRGSGSGTSYSLTISRME |
| | SEDTAVYYCASSQPFTYWGQGTLVTVSA | AEDGATYYCQQWTSFPPTFGAGTKL |
| | (SEQ ID NO: 389) | ELK (SEQ ID NO: 437) |
| TM-21 | EVQLVESGGGLVKPGGSLKLSCAASGFTF | DIVMTQAAFSNPVTLGTSASISCRSSK |
| | SNYAMSWVRETPEKRLEWVAFISDGGGY | SLLHSNGITYLFWYLQKPGQSPQLLI |
| | IYYADNVKDRFTISRDNAKNNLYLQMRH | YQMSNLASGVPDRFSSSGSGTDFTLR |
| | LKSEDTAMYYCARDGGTGFTYWGQGTL | ISRVEAEDVGVYYCVQNLELPYTFG |
| | VTVSV (SEQ ID NO: 393) | GGTKLEIK (SEQ ID NO: 438) |
| TM-22 | QVQLQQSGPELVKPGASVKISCKASGYRF | DVLMTQTPLSLPVSLGDHASISCRSG |
| | TSYYIHWVKQRPGQGLEWIGWIYPGNGIT | QSIVHSNGNTYLEWYLQKPGQSPKL |
| | NYNEKFKGKATLTADTSSSTAYMQLSSLT | LIYKVSNRFSGVPDRFSGSGSGTDFTL |
| | SEDSAVYYCASPYYGIRNCYFDVWGTGT | KISRVEAEDLGIYYCFQGSHVPWTFG |
| | TVTVSS (SEQ ID NO: 394) | GGTKLEIK (SEQ ID NO: 439) |
| TM-23 | QVQLQQSGAELIRPGTSVKVSCKASGYAF | DIVVTQTPLSLPVSFGDQVSISCRSSQ |
| | TNYLIEWVKKRPGQGLEWIGVINPGSGIT | SLTNYYGNTYLSWYLHKPGQSPQLLI |
| | NYNEKFKGKATLTADKSSSTAYMQLSSL | YGISNRFSGVPDRFSGSGSGTDFTLKI |
| | TSEDSAVYFCARSDFITTVVADYWGQGTT | STIKPEDLGMYYCLQGTHQPRTFGG |
| | VTVSS (SEQ ID NO: 395) | GTKLEIK (SEQ ID NO: 440) |
| TM-24 | QVQLKQSGPGQVQPSQSLSITCTVSGFSLS | DIVMTQSPSSLTMSVGQKVTMNCKS |
| | DYGVHWVRQSPGKGLEWLGVIWNNGNT | SQSLLNSNNQQNYLAWYQQKPGQSP |
| | DYNAAFISRLSINKDNSKSQVFFKMTSLQ | KLLVYFASIRESGVPDRFIGSGSGTDF |
| | ADDTAIYYCVRSLRPLHFDYWGQGTTVT | TLTINSVQAEDLADYFCQQHYSTPFT |
| | VSS (SEQ ID NO: 384) | FGSGTKLEIR (SEQ ID NO: 441) |
| TM-25 | EVQLVESGGDLVKPGGSLRLSCAASGFTF | DIVMTQAAFSNPVTLGTSASISCRSSK |
| | SGYGMSWIRQTPDKRLEWVATISSGGRY | SLLHSNGITYLFWYLQKPGQSPQLLI |
| | TVYPDSVKGRFTMSRDNVKNTLYLQMSS | YQMSNLASGVPDRESSSGSGTDFTLR |
| | LKSEDTALYYCARDNFYSYAMDYWGLG | ISRVEAEDVGVYYCAQNLELWTFGG |
| | TSVTVSA (SEQ ID NO: 396) | GTKLEIK (SEQ ID NO: 442) |
| TM-26 | QVQLQQSGAELIRPGTSVKVSCKASGYAF | DIVVTQTPLSLPVSFGDQVSISCRSSQ |
| | TNYLIEWVKKRPGQGLEWIGVINPGSGIT | SLTNYYGNTYLSWYLHKPGQSPQLLI |
| | NYNEKFKGKATLTADKSSSTAYMQLSSL | YGISNRFSGVPDRFSGSGSGTDFTLKI |
| | TSEDSAVYFCARSDFITTVVADYWGQGTT | STIKPEDLGMYYCLQGTHQPRTFGG |
| | VTVSS (SEQ ID NO: 395) | GTKLEIK (SEQ ID NO: 440) |
| TM-27 | QVQLQQSGAELVRPGTSVKVSCKASGYA | QIVLTQSPAIMSASPGEKVTISCSASS |
| | LTNYLIEWVKQRPGQGLEWIGVINPGSGS | SISYMYWYQQKPGSSPKPWIYRTSTLA |
| | TKYNEKFKGKATLTADKSSSTAYMQLSS | SGVPARFSGSGSGTSYSLTISSMEAED |
| | LTSEDSAVYFCARIIYDHDWYFDVWGTG | AATYYCQQYHSYPRTFGGGTKLEIK |
| | TTVTVSS (SEQ ID NO: 397) | (SEQ ID NO: 443) |
| TM-28 | EVQLQQSGPVLVKPGPSVKISCMASVFTF | DIVMTQSPSSLSVSVGEKVTVSCKSS |
| | NDYYIHWVKQSHGKSLEWIGLVYPYNGG | QSLLNSGNQKNYLAWYQQKPGQPP |
| | TNYNQNFKGKATLTVDTSSRTAYMELNS | KLLIYGASTRESGVPDRFTGSGSGTD |
| | LTSEDSAVYYCARSYFSNPIGYWGQGTLV | FTLTISSVQAEDLAVYYCQNDHSYPL |
| | TVSE (SEQ ID NO: 398) | TFGAGTKLELK (SEQ ID NO: 444) |
| TM-29 | QVQLRQSGAELVRPGASVKLSCKASGYT | DIVMTQAAFSNPVTLGTSASISCRSSK |
| | FTDYYINWVKQRPGQGLEWIARIYPGSGY | SLLHYNGITYLYWYLQKPGQSPQLLI |
| | TYYNEKFKGKATLTAEGSSNTAYMQLSS | YQMSNLASGVPDRFSSSGSGTDFTLR |
| | LTSEDSAVYFCANHYTNPFAYWGQGTLV | ISRVEAEDVGVYYCAQNLELPYTFG |
| | TVSA (SEQ ID NO: 399) | GGTKLEIK (SEQ ID NO: 445) |
| TM-30 | QVQLQQSGPELVKPGASVKLSCKASDNT | DVLMTQSPLSLPVSLGDQVSISCRSS |
| | FTNYDVNWVRQRPGQGLEWIGWIYPRDG | QTIVHRNGNTYLEWYLQKPGQSPKL |
| | TTIYNEKFKGRATLTVDTSSSTAYMELHS | LIYKVSNRFSGVPDRFSGSGSGTDFTL |
| | LTSEDSAVFFCARTLPQAMDYWGQGTSV | KISRVEAEDLGVYYCFQGSHLPWTF |
| | TVSS (SEQ ID NO: 400) | GGGTKLEIK (SEQ ID NO: 446) |
| TM-31 | QVQLQQSGAELAKPGASVKVSCKASGYT | DIKMTQSPSSMYASLGERVTFTCKAS |
| | FISYWMNWVKQRPGQGLEWIGYINPTSG | QDINSYLSWFQQKPGKSPKTLIYRGN |
| | YTRYNQKFKDKATLTADKSSSTAYMQLS | GLVDGVPSRFSGSGSGQDYSLTISSLE |
| | SLTYEDSAVYYCARSPPTVVLIGYFDYWG | YEDMGIYYCLQYDEFPFTFASGTKLE |
| | QGTTLTVSS (SEQ ID NO: 401) | IK (SEQ ID NO: 447) |
| TM-32 | EVQLQQSGAELVRPGASVRLSCIASGFNI | DVLMTQTPLSLPVSLGDQASISCRSS |
| | KDYYMYWVKQRPEQGLEWIGRIDPEDGD | QNIVHSNGNTYLEWYLQKPGQSPKL |
| | TEYVPKFQGKATMTADTSSNTAYLQLSSL | LIDKVSNRFSGVPDRFSGSGSGTDFTL |
| | TSEDIGVYYCTTRTWDLYYAVDNWGQG | KISRVEAEDLGIYYCFQGSHVPFTFGS |
| | TSVTVSS (SEQ ID NO: 402) | GTKLEIK (SEQ ID NO: 448) |
| TM-33 | EFQLQQSGPELVKPGASVKISCKASGYSF | DIVMTQSQKFMSTTVGDRVSITCKAS |
| | TDYNMNWVKQSNGKSLEWIGVINPNYGT | QNVGTAVAWYQQKPGQSPKLLIYSA |
| | TSYNQKFKGKATLTVDQSSSTAYMQLNS | SNRYTGVPDRFTGSGSGTDFTLTISN |
| | LTSEDSAVYYCASSYWGQGTLVTVSA | MQSEDLADYFCQQYSSYPYTFGGGT |
| | (SEQ ID NO: 403) | KLEIK (SEQ ID NO: 449) |
| TM-34 | EVQLVESGGGLVKPGGSLKLSCAASGFSF | QIVLSQSPVILSASPGEKVTMTCRATS |
| | SDSGMDWVRQAPEKGLEWFAYISSGSST | SVTYMHWYQLKPGSSPKPWIYATSN |
| | THYADTVKGRFIISRDNAKNTLFLQMTSL | LASGVPARFSGSGSGTSYSLTISRVEA |
| | RSEDTAMYYCVRRDGNYWYFDVWGTGT | EDAATYYCQQWSSNPYTFGGGTKLE |
| | TVTVSS (SEQ ID NO: 404) | IK (SEQ ID NO: 450) |
| TM-35 | QVQLQQSGPELVRPGTSVKISCKAPGYTF | DVLMTQIPLSLPVSLGDQASISCRSSQ |
| | TRHWMQWVRQRPGQGLEWIGEILPGSNN | SIVHRNGNTYLEWYLQKPGQSPKLLI |
| | IYYNEKVKGKATLTVDTSSSTSYMQLSSL | YKVSNRFSGVPDRFSGSGSGTDFTLK |
| | TSEDSAVYFCARSLYDYDGVFAYWGQGT | ISRVEAEDLGVYYCFQGSHVPYTFGG |
| | LVTVSA (SEQ ID NO: 405) | GTKLEIK (SEQ ID NO: 451) |
| TM-37 | QVQLQQSGAELAKPGASVKLSCKASGYT | DIVMTQAAPSVPVTPGESVSISCRSSK |
| | FTSYWMHWVKQRPGQGLEWIGYVNPSS | SLLHSNGNTYSYWFLQRPGQSPQLLI |
| | GYTKNNQKFKDKVTLTADKSSSTAYMQL | YRMSNLASGVPDRFSGSGSGTAFTLR |
| | SSLTYEDSAVYYCAREGGSISDWYFDVW | ISRVEAEDVGVYYCMQHLEYPYTFG |
| | GTGTTVTVSS (SEQ ID NO: 406) | GGTKLEIK (SEQ ID NO: 452) |
| TM-39 | HVQRQQSGTELAKPGASVKLSCKTSGYT | DIVMTQAAPSIPVTPGESVSISCRSSK |
| | FTDYWMHWIKQRPGQGLEWIGFINPSSG | TLLNSNGNTYLYWFLQRPGQPPQLLI |
| | YTKYNQNFKDKATLTADKSSSTAYMQLS | YRMSNLASGVPDRFSGSGSGTAFTLR |
| | SLTYEDSAVFYCAREAGSISDWYFDVWG | ISRVEAEDVGVYYCMQHLDYPYTFG |
| | TGTTVTVSS (SEQ ID NO: 407) | GGTQLEIK (SEQ ID NO: 453) |
| TM-41 | EVQLQQSGAELVKPGASIKLSCTASGFNIK | DIVMSQSPSSLAVSVGQKVTMSCKSS |
| | DCYMHWVKQRTEQGLEWIGRIDPEDGTT | QSLLYSSNQKNYLAWYQQKPGQSPK |
| | NFAPKFQDRATITADTSSNTAYLQLTSLTS | LLIYWASTRESGVPDRFTGSGSGTDF |
| | EDTAVYYCAREWDSGAYWGQGTLVTVS | TLTISSVKTEDLAVYYCQQYYSYPYT |
| | A (SEQ ID NO: 408) | FGGGTKLEIK (SEQ ID NO: 454) |
| TM-42 | QVQLQQSGAELAKPGASVKLSCKASGYT | DIVMTQAAPSLPVTPGESVSISCRSSK |
| | FTNYWMHWVKKRPGQGLEWIGYINPSSG | SLLHSNGNTYLYWFLQRPGQSPQLLI |
| | YTKYNQKFKDKATLTADKSSNTAYMQLS | YRMSNLASGVPDRFSGSGSGTAFTLR |
| | SLTYEDSAVYYCTREGGSISDWYFDVWG | ISRVEAEDVGVYYCMQHLEYPYTFG |
| | TGTTVTVSS (SEQ ID NO: 409) | GGTKLEIK (SEQ ID NO: 455) |
| TM-44 | EVQLVESGGGLVKPGGSLKLSCAASGFTF | DIVMSQSPSSLAVSVGEKVTMSCKSS |
| | SDYGMHWVRQAPEKGLEWVAYISSGSST | QSLLYSSNQKNYLAWYQQKPGQSPK |
| | IYYADTVKGRFTISRDNAKNTLFLQMTSL | LLIYWASTRESGVPDRFTGSGSGTDF |
| | RSEDTAMYYCARNYGSPYAMDYWGQGT | TLTISSVKAEDLAVYYCQQYYSYPTF |
| | SVTVSS (SEQ ID NO: 410) | GGGTKLEIK (SEQ ID NO: 456) |
| TM-45 | QVQLQQSGAELAKPGASVKLSCKASGYT | DIVMTQAAPSLPVTPGESVSISCRSSK |
| | FTNYWMHWVKKRPGQGLEWIGYINPSSG | SLLHSNGNTYLYWFLQRPGQSPQLLI |
| | YTKYNQKFKDKATLTADKSSNTAYMQLS | YRMSNLASGVPDRFSGSGSGTAFTLR |
| | SLTYEDSAVYYCTREGGSISDWYFDVWG | ISRVEAEDVGVYYCMQHLEYPYTFG |
| | TGTTVTVSS (SEQ ID NO: 409) | GGTKLEIK (SEQ ID NO: 455) |
| TM-46 | QVQLQQSGADLVKPGASVKISCKASGYA | QIVLTQSPVIMSASPGERVTMTCCAS |
| | FSSFWMNWVKLRPGKGLEWIGQIYPGDG | SRVNYMHWYQQKSGSYPKRWIYDT |
| | DTDYNGKFKDKATLTADTSSNTAYMQLS | SKLASGVPGRFSGSGSGTSYSLTISSM |
| | RLTSEDSAVYFCARGDGFSYFDYWGQGT | EAEDAATYYCQQWSSNPPTFGGGTK |
| | ILTVSS (SEQ ID NO: 411) | LEIK (SEQ ID NO: 457) |
| TM-47 | QVQLKESGPVLVAPSQSLSITCTVSGFSLT | DIVMTQSPSSLTVTAGEKVTMSCKSS |
| | NYGVHWVRQPPGKGLEWLGVIWAGGNT | QSLLNSGNQKNYLTWYQQKPGQPPK |
| | NYNSALMSRLSISKDNSKSQVFLKMNSLQ | LLIYWASTRESGVPDRFTGSGSGTDF |
| | TDDTAMYYCAKEAKLLRSYAMDYWGQ | TLTISSVQAEDLAVYYCQNDYSYPLT |
| | GTSVTVSS (SEQ ID NO: 412) | FGAGTKLEIK (SEQ ID NO: 458) |
| TM-48 | QVQLQQSGPELVKPGASVKLSCKASGYT | DVLMTQTPLSLPVSLGNQASISCRSS |
| | FTSYDINWVKQRPGQGLEWIGWIYPRDG | QSIVHGNGNTYLEWYLQKPGQSPKL |
| | NTQYIEKLKGKATLTVDTSSSTAYMELHS | LIYKVSNRFSGVPDRFSGSGSGTDFTL |
| | LTSEDSAVYFCARWIFYAMDYWGQGTSV | KISRVEAEDLGVYYCFQGSHLPYTFG |
| | TVSS (SEQ ID NO: 413) | GGTKLEIK (SEQ ID NO: 459) |
| TM-49 | QVQLQQPGAELVRPGSSVKLSCKASGYTF | QIVLTQSPAIMSASPGEKVTISCSASSS |
| | TRFWMHWVKQRPIQGLEWIGNIDPSDSQ | VSYMYWYQQKPGSSPKPWIHRTSNL |
| | THYNQKFKDKATLTVDKSSSTAYMQLSS | ASGVPVRFSGSGSGTSYSLTISSMEAE |
| | LTSEDSAVYYCARLITVDYAMDYWGQGT | DAATYYCQQYHSYPPTFGAGTKLEL |
| | SVTVSS (SEQ ID NO: 414) | K (SEQ ID NO: 460) |
| TM-50 | QVQLKESGPVLVAPSQSLSITCTVSGFSLT | DVQMTQTTSSLSASLGDRVTISCRAS |
| | NYGVHWVRQPPGKGLEWLGVIWAGGNT | QDINNYLYWYQQKPDGTVKLLIYYT |
| | NYNSALMSRLSISKDNSKSQVFLKMNSLQ | SMLHSGVPSRFSGSGSGTDYSLTISNL |
| | TDDTAMYYCAKEAKLLRSYAMDYWGQ | EQEDIATYFCQQGSTLMYTFGGGTK |
| | GTLVTVSA (SEQ ID NO: 415) | LEIK (SEQ ID NO: 461) |
| TM-51 | QVQLQQPGAELVKPGASVKLSCKASGYT | DIVMSQSPSSLAVSVGEKVTMSCKSS |
| | FTTYWIDWVKQRPGQGLEWIGNMFPGSS | QSLRNSRTRKNYLAWYQQKPGQSPK |
| | RTNYNEKFKSRATLTVDTSSSTAYMQLSS | LLIYWASTRESGVPDRFTGSGSGTDF |
| | LTSDDSAVYYCARKEGLWTYGYDGGAW | TLTISSVQAEDLAIYYCKQSYNLLTF |
| | FAYWGQGTLVTVSA (SEQ ID NO: 416) | GAGTKLELK (SEQ ID NO: 462) |
| TM-52 | QVQLQQPGAELVKPGASVKLSCKASGYT | ENVLTQSPAIMSASPGEKVTMTCSAS |
| | FTNYWMHWVKQRPGQGLDWIGNIDPSDS | SSVSYMHWYQQKSNTSPKLWIYDTS |
| | ETHYNQKFKDKATLTVDKVSSTAYMQLS | KLASGVPGRFSGSGSGNSYSLTISSAE |
| | SLTSEDSAVYYCARRGYYGRSPFAYWGQ | AEDVATYYCFQGSGYPLTFGAGTKL |
| | GTLVTVSA (SEQ ID NO: 417) | ELK (SEQ ID NO: 463) |

TABLE 6

| Antibody | Variable Heavy Chain | Variable Light Chain |
|---|---|---|
| TM-54 | QVQMQQSGPVLVKPGASVTLSCKASGYT | DVLMTQTPLSLPVSLGDQASISCRSS |
| | FTTYDLNWVKQRPGQGLEWIGWIYPRGG | QSIVHGNGNTYLEWYLQKPGQSPK |
| | STEYNEKFKGKATLTVDTSSSTAYMELHS | LLIYKVSNRFSGVPDRFSGSGSGTDF |
| | LTSEDSAVYFCAKYSIYAMDYWGQGTSV | TLKISRVEAEDLGVYYCFQGSHVPWT |
| | TVSS (SEQ ID NO: 464) | FGGGTKLEMK (SEQ ID NO: 500) |
| TM-56 | QVHLQQSGAELAKPGASVKLSCKASGYT | DIEMTQAAPSVPVTPGESVSISCRSS |
| | FTSYWMHWVKQRPGQGLEWIGYINPSSG | KSLLQNNGNTYLYWFLQRPGQPPQ |
| | YSKYNQRFKDKATLTADKSSTTAYMHLS | LLIYRMSNLASGVPDRFSGSGSGTA |
| | SLTYADSAVYYCTREGGSISDWYFDVWG | FTLRISRVEAEDVGVYYCMQHLEYP |
| | TGTTVTVSS (SEQ ID NO: 465) | YTFGGGTKLEIK (SEQ ID NO: 501) |
| TM-59 | QVQLQQSGAELAKPGASVKLSCKASGYT | DIVMTQAAPSVPVTPGESVSISCRSS |
| | FISYWMHWVKQRPGQGLEWIGYVNPSSG | KSLLHSNGNTYSYWFLQRPGQSPQL |
| | YTKNNQKFKDKATLTADKSSSTAYMQLS | LIYRMSNLASGVPDRFSGSGSGTAF |
| | SLTYEDSAVYYCAREGGSISDWYFDVWG | TLRISRVEAEDVGVYYCMQHLEYP |
| | TGTTVTVSS (SEQ ID NO: 466) | YTFGGGTKLEIK (SEQ ID NO: 452) |
| TM-60 | QVQLQQSGPELVKPGASVKLSCKASGYT | DVLMTQTPLSLPVSLGDQASISCRSS |
| | FTSYDINWVKQRPGQGLEWIGWIYPRDG | QSIVHGNGNTYLEWYLQKPGQSPK |
| | NTQYIEKLKGKATLTVDTSSSTAYMELHS | LLIYKVSNRFSGVPDRFSGSGSGTDF |
| | LTSEDSAVYFCARWIFYAMDYWGQGTSV | TLKISRVEAEDLGVYYCFQGSHLPY |
| | TVSS (SEQ ID NO: 413) | TFGGGTKLEIK (SEQ ID NO: 502) |
| TM-61 | QVQLQQSGAELAKPGASVKLSCKASGYT | DIVMTQSPSSLSASAGEKVTMSCKS |
| | FTNYWMNWVKQRPGQGLEWIGYINPSSD | SQSLLNSGNQKNYLAWYQQKPGQS |
| | YTKYNQNFKDKATLTADKSSSTAYMQLN | PKLLIYGASTRESGVPDRFTGSGSGT |
| | SLTYEDSAVYYCTRWGGSRSYWYFDVW | DFTLTISSVQAEDLAVYYCQNDSNY |
| | GAGTTVTVSS (SEQ ID NO: 467) | PFTFGGGTKLEMK (SEQ ID NO: 503) |
| TM-62 | QVQLQQPGTELVRPGSSVKLSCKAAGYT | QIVLTQSPAIMSASPGEKVTISCSASS |
| | FTRYWMHWVKQRPIQGLEWIGNIDPSDS | SVSYMYWYQQKAGSSPKPWIHRTS |
| | ETQYNPKFKDKATLTVDRSSSTAYMHLS | NLASGVPARFSGSGSGTSYSLTIRSM |
| | SLTSEDSAVYYCARLTTLDYAMDYWGQ | EAEDAATYYCQQYHSYPPTFGVGT |
| | GTSVTVSS (SEQ ID NO: 468) | KLELK (SEQ ID NO: 504) |
| TM-63 | QVQLQQPGAELVKFGASVKLSCKASGYT | DIVMTQASPSVSVTPGESVSISCRSS |
| | FTSFWMHWVKQRPGQGLEWIGNIDPSDS | QSLLHVNGNTYLYWFLQRPGRSPR |
| | ETHYSQKFKDKATLTVDRSSNTAYIQLSS | LLIYRMSNLASGVPDRFSGSGSGTA |
| | LTSEDSAVYYCARSSNWDNFDYWGQGT | FTLRISRVEAEDVGVYYCMQHLQY |
| | TVTVSS (SEQ ID NO: 469) | PFSFGSGTKLEMK (SEQ ID NO: 505) |
| TM-64 | QVQLQQPGAELVKPGASVKLSCKASGYT | DIVMTQAAPSVPVTPGESVSISCRSS |
| | FTSYWMHWVKQRPGHGLEWIGNIDPSDS | KSLLHTNGNTYLFWFLQRPGRSPQL |
| | DTHYNQNFRGKATLTVDKFSTTAYMHLS | LIYRMSNLASGVPDRFSGSGSGTAF |
| | SLTSEDSAVYYCARSRGGTYDYGEAMDY | TLRISRVEAEDVGVYYCMQHLEYPFT |
| | WGQGTSVTVSS (SEQ ID NO: 470) | FGSGTKLEIK (SEQ ID NO: 506) |
| TM-65 | QVQLQQPGAEFVKPGASVRLSCKTSGYT | DIQMNQSPSSLSASLGDTITITCRAS |
| | FTSSWMHWVKQRPGQGLEWIGEINPKNG | QNINIWLSWYQQKPGNVPKLLIYKA |
| | GTNYNEKFKTKATLTVDKSSNTAYMQLS | SNLHTGVPSRFSGSGSGTGFTLTISS |
| | SLTSEDSAVYYCTIPTWFTYWGQGTLVT | LQPEDIATYYCLQGQSYPYTFGGGT |
| | VSS (SEQ ID NO: 471) | KLGIK (SEQ ID NO: 507) |
| TM-66 | EVMLVESGGALVEPGGSLKLSCAASGITF | DVVVTQTPPSLPVSFGDQVSISCRSS |
| | SNYAMSWIRQTPEKRLEWVATIGSVGSY | QSLVNSYGKTFLSWYLHKPGQSPQL |
| | TYYPDSVKGRFTISRDNARNTLYLQMSSL | LIYGISNRFSGVPDRFSGSGSGTVFT |
| | RSEDTAMYYCARQEGSSHFDYWGQGTT | LKISTIKPEDLGMYYCLQGTHPPLTF |
| | LTVSS (SEQ ID NO: 472) | GAGTKLEMK (SEQ ID NO: 508) |
| TM-68 | EVQLQQSGAELVRPGASVKLSCTASGFNI | DIVMTQAAFSNPVTLGTSVSISCSSS |
| | KDDYMHWVRQRPEQGLEWIGWIDPENG | KSLLHSSGITYLYWYLQRPGQSPQL |
| | DTEYASKFQGKATMTADTSSNTAYLHLS | LIYRMSNLASGVPDRFSGSGSGTDF |
| | SLTSEDTAVYYCTKPQTATTCWGQGTLV | TLRISRVEAEDVGVYYCAQMLERP |
| | TVSS (SEQ ID NO: 473) | WTFGGGTKLEIK (SEQ ID NO: 509) |
| TM-69 | QVQLKESGPDLVQPSQTLSLTCTVSGFSF | DVVMTQTPLSLPVSLGDQASISCRSS |
| | TSYGVHWVRQPPGKGLEWVGIMGWDDK | QSLVHSNGNTYLHWYLQKPGQSPK |
| | KYYNSALKSRLSISRDTSKNQVFLKLSSL | LLIYKVSNRFSGVPDRFSGSGSGTDF |
| | QTEDTAMYYCTREAGPYGMDYWGQGTS | TLKISRVEAEDLGVYFCSQNTHVPL |
| | VTVSS (SEQ ID NO: 474) | TFGAGTKLELK (SEQ ID NO: 510) |
| TM-70 | EVQLVESGGALVKPGGSLKLSCAASGFTF | DIVLTQSPASLAVSLGQRATISCRAS |
| | SNYAMSWVRQTPEKRLEWVATIGYGGS | ESVDNYGISFMNWFQQKPGQPPKLL |
| | YSYYPDSVKGRFTISRDNAKYTLYLQMS | IYAASNQGSGVPARFSGSGSGTDFS |
| | GLRSEDTALYYCARQDSNYEGVWFAYW | LNIHPMEEDDTAMYFCQQSKDVPW |
| | GQGTLVT (SEQ ID NO: 475) | TFGGGTKLAIK (SEQ ID NO: 511) |
| TM-71 | EVQLVESGGALVKPGGSLKLSCAASGFTF | DIVMTQAAPSVPVTPGESVSISCRSS |
| | SNYAMSWVRQTPEKRLEWVATIGYGGS | KSLLHTNGNTYLFWFIQRPGQSPHL |
| | YSYYPDSVKGRFTISRDNAKNTLYLQMSS | LIYRKSNLASGVPDRFSGSGSGTAFT |
| | LRSEDTALYYCARQDSNYEGVWFAYWG | LRISRVEAEDVGVYYCMQHLEYPFT |
| | QGTLVTVSS (SEQ ID NO: 476) | FGSGTKLEIK (SEQ ID NO: 512) |
| TM-72 | QVQLQQPGAELVKPGTSVKLSCKASGSTF | DIVMTQAAPSVPVTPGESVSISCRSS |
| | TNYWMHWVMQRPGQGLEWIGNIDPSDS | KSLLHTNGNTYLFWFIQRPGQSPHL |
| | DTHYNQKFKDRAKLTVDKSSSTAYMQLS | LIYRKSNLASGVPDRFSGSGSGTAFT |
| | GLTSEDSAVYYCARSRGGYYDYGEAMDY | LRISRVEAEDVGVYYCMQHLEYPFT |
| | WGQGTSVTVSS (SEQ ID NO: 477) | FGSGTKLEIK (SEQ ID NO: 512) |
| TM-73 | QVQLQQPGAELVKPGASVKLSCKASGYT | QIVLTQSPAIMSASPGEKVTLTCSAS |
| | FTSHWMHWVKHRPGQGLEWIGEIDPSDS | SGISYIYWYQQRPGSSPRLLIYDTSN |
| | YTYYNQKFKGKATLTVDKSSSTVYMQLS | LASGVPVRFSGSGSETSYSLTISRLE |
| | SLTSEDSAVYYCARNYHGSGPPFAHWGQ | AEDAATYYCHQWRSYPPTFGGGTK |
| | GTLVTVSS (SEQ ID NO: 478) | LEIR (SEQ ID NO: 513) |
| TM-74 | EVQLQQSGPELVKPGASVKMSCKASGYT | DVVMTQSQKFMSTSVEDRVSITCK |
| | FTSYVIHWVKQKPGQGLEWIGFINPNNDG | ASQNVRSAVAWYQQKPGQSPKALI |
| | TKYNEKFRGKATLTSDKSSSTAYMELSSL | YLASNRHTGVPDRFAGSGSGTDFTL |
| | TSEDSAVYYCARPYYDYEGYTMDYGGQ | TISSVQSEDLADYFCLQHWKYPFTF |
| | GTSVTVSS (SEQ ID NO: 479) | GSGTKLEIK (SEQ ID NO: 514) |
| TM-75 | EVQLQQSGPELVKPGASVKMSCKASGYT | DIVLTQSPASLAVSLGQRATIFCRAS |
| | FTSYVMHWVKQKPGQGLEWIGYINPYND | QSVDYNGISYMHWFQQKPGQPPKL |
| | GTKYNEKFKGKATLTSDKSSSTAYMELSS | LIYAASNLESGIPARFSGSGSGTDFT |
| | LTSEDSAVYYCARGGWFPYAMDYWGQG | LNIHPVEEEDAATYYCQQSIEDPWT |
| | TTVTVSS (SEQ ID NO: 480) | FGGGTKLEIK (SEQ ID NO: 515) |
| TM-76 | QVTLKESGPGILQPSQTLSLTCSFSGFSLS | QIILTQSPAIMSASLGEEITLTCSASL |
| | TSGMGVSWIRKPSGKGLEWLAHIFWDDDK | SISYVHWYQQKSGASPKLLIYGTSN |
| | RYNLFLKSRLTVSKDTSSNQVFLMITSVD | LASGVPSRFSGSGSGTFYSLTISSVE |
| | TTDTATYYCARMGRQRDYFDYWGQGTS | AEDAADYYCHQWSIYRTFGGGTKL |
| | LTVSS (SEQ ID NO: 481) | EMK (SEQ ID NO: 516) |
| TM-77 | QIQLQQSGAELVRPGASVKLSCKASGYTF | DVVMTQIPLTLSVTLGQPASISCKSS |
| | TDYEMHWVKQTPVHGLEWIGVIDPETGG | QSLFHSNGKTYLNWLLQRPGQSPKL |
| | TAYNQMFKGKATLTADKSSSTAYMDLRS | LIYMVSKLESGVPDRFSGSGSGTDF |
| | LTSEDSAVYYCVKYGIEGYWGQGTTLTV | TLKISRVEAEDLGVYYCLQVTHFPL |
| | SS (SEQ ID NO: 482) | TFGAGTKLELK (SEQ ID NO: 517) |
| TM-78 | QVQLQQPGAELVKPGASVKLSCKASGYT | DIVMTQATPSIPVTPGESVSISCRSSK |
| | FTNHWMHWVRQRPGQGLEWIGNIDPSDS | SLLHVNGNTYLFWFLQRPGQSPQLL |
| | DTHYNHKFKDKATLIVDKSSSTAYLQLSS | IYRMSNLASGVPDRFSGSGSGTAFT |
| | LTSEDSAVFYCARSRGGYYDYGEAMDY | LRISRVEAEDVGVYYCMQHLEYPFT |
| | WGQGTSVTVSS (SEQ ID NO: 483) | FGSGTKLEIK (SEQ ID NO: 518) |
| TM-79 | EVQLQQSGPELVKPGASVKMSCKASGYT | DIVLTQSPASLAVSLGQRATISCRAS |
| | FTNYVMHWLKQKPGQGLEWIAHITPHND | KSVSTSGSIYIHWYQQKPGQPPKLLI |
| | GTKYNEKFKGKATLTSDRSSSTAYMELN | YLTSNLESGVPARFSGSGSGTDFTL |
| | SLTSEDSAIYYCARSGQFGRGDYFHYWG | NIHPVEEEDAATYYCQHSGELPWTF |
| | QGTTVTVSS (SEQ ID NO: 484) | GGGARLEIK (SEQ ID NO: 519) |
| TM-80 | QVQLQQPGAELVKPGASVKLSCKASGYT | DIVMTQAAPSVPVTPGESVSISCRSS |
| | FTTFWMHWVKQRPGQGLEWIGNIDPSDS | QSLLHVNGHTYLYWFLQRPGQSPQ |
| | ETHYNQEFKDKATLTVDRSSNTAYMQLS | LLIYRMSNLASGVPDRFSGSGSGTV |
| | SLTSEDSAVYYCARSSNWDNFDYWGQG | FTLRISRVETEDVGIYYCMQHLQYP |
| | TAVTVSS (SEQ ID NO: 485) | FTFGSGTKLEIK (SEQ ID NO: 520) |
| TM-81 | QVQLQQSGAELVKPGASVKLSCKASGYT | DIVMTQSPSSLAMSVGQKVTMSCK |
| | FTSYWMHWVKQRPGQGLEWIGNIDPSDS | SSQSLLNSKNQKNYLAWYQQKPGQ |
| | EIHYNQKFKDKATLTVDKSSSTAYIQLSS | SPKFLVYFAFFRESGVPDRFIGSGSG |
| | LTSEDSAVYYCARSPAGQILDYWGQGTT | TDFTLTISSVQAEDLADYFCQQHYS |
| | VTVSS (SEQ ID NO: 486) | TPFTFGSGTKLEIK (SEQ ID NO: 521) |
| TM-82 | QVQLQQSGAELVRPGTSVRVSCKASGYA | DVVVTQTPLSLPVSFGDQVSISCRSS |
| | FTNYLIEWIKQRPGQGLEWIGVINPGSGIT | QSLANTYGNTYLSWYLHTPGQSPQ |
| | DYSEKFKGKARLTADKSSSTAYMQLSSL | LLIFGISNRFSGVPDRFSGSGSGTDFT |
| | TSDDSAVYFCTRSPFITTVVADYWGQGTT | LKISTIKPEDLGVYYCLQGTHPPRTF |
| | VTVSS (SEQ ID NO: 487) | GGGTKLEMK (SEQ ID NO: 522) |
| TM-83 | QVQLQQPGTELVKPGALVKLSCKASGYT | DVVMTQTPLSLPVSLGDQASISCRSS |
| | FTTYWIHWVKQRPGQGLEWIGNINPSNG | QSLVHSNGITYLHWYLQKPGQSPKL |
| | NTKNNERFKTKATLTVDKSSSTAYMQLS | LIYKVSNRFSGVPDRFSGSGSGTDFT |
| | SLTSEDSAVYYCVRGDYYGTSYPFWGQG | LKISRVEAEDLGVYFCSQSTHVPYT |
| | TLVTVSS (SEQ ID NO: 488) | FGGGTKLEIK (SEQ ID NO: 523) |
| TM-84 | QVQLQQSGAELVKPGASVKMSCKASGYT | DIQMTQSPASLSVSVGETVTITCRAS |
| | FTTFPIEWMKQNHGKSLEWIGNFHPYND | ENIYSSLGWYHQKQGKSPQLLVFA |
| | DTKYNEKFKGKATLTVEKSSSTVYLELSR | ATNLADGVPSRFSGSGSGTQYSLKI |
| | LTSDDSAVYYCARYFYGGMDYWGQGTS | NSLQSEDFGTYYCQHLWSVPWTFG |
| | VTVSS (SEQ ID NO: 489) | GGTKLEIK (SEQ ID NO: 524) |
| TM-85 | QVQLQQPGAELVRAGTSVKLSCKASGYT | DIAMSQSQKFMSTSVGDRVSVTCK |
| | FIGYWMHWVKQRPGRGLEWIGRIDPYSG | ASQNVGTNVAWYQQKPGQSPKALI |
| | DTRYNEKFKNKATLTVDKPSSTAYMQLS | YSASYRYSGVPDRFTGSGSGTDFTL |
| | SLTSEDSAVYYCARYGLGHAMDYWGQG | TISNVQSEDLAEYFCQQYNSYPLTF |
| | TSVTVSS (SEQ ID NO: 490) | GAGTKLEMK (SEQ ID NO: 525) |
| TM-86 | EVQLQQSGPVLVKPGASVKMSCKASGYT | DIVMTQSPSSLSVSAGEKVTMSCKS |
| | FTDYYIHWVKQSHGRSLEWIGVISPYNDN | SQSLLNSGNQKNYLAWHQQRPGQP |
| | TNYNQKFKGKATLTVDKSSSTAYMELNS | PKLLIYGASTRESGVPDRFTGSGSGT |
| | LTSEDSAVYYCVRTYYSNYFDYWGQGTT | EFTLTISSVQAEDLAVYYCQNDHSY |
| | LTVSS (SEQ ID NO: 491) | PLTFGAGTKLELK (SEQ ID NO: 526) |
| TM-87 | EVQLQQSGPVLVKPGASVKLSCKASGYT | DVQITQSPSYLAASPGETITINCRAS |
| | FTDYYMNWVKQSHGKSLEWIGVINPYNG | RSISKYLAWYQEKPGKTNKLLIYSG |
| | ATSYNQKFQDKATLTVDKSSSTAYMGLN | STLQSGIPSRFSGSGSGTDFTLTISSL |
| | SLTSEDSAVYYCANGNYDWYFDVWGTG | EPEDFAVYYCQQHNEYPLTFGAGT |
| | TTVTVSS (SEQ ID NO: 492) | KLELK (SEQ ID NO: 527) |
| TM-88 | EAQLQQSGPVLVKPGASVKMSCKTSGYT | ETTVTQSPASLSMAIGEKVTIRCITSS |
| | FTDYYINWVKQSHGKSLEWIGVINPYSGA | DIDDHMNWYRQKPGEPPEFLISEGN |
| | TYYTQKFKGKATLTVDKSSSTAYMELNS | ALRPGVPSRFSSSGYGTDFIFTIENIL |
| | QTSEDSAVYYCARGGYDYGHWYFDVW | SEDVADYYCLQSDNLPLTFGAGTKL |
| | GTGTTVTVSS (SEQ ID NO: 493) | ELK (SEQ ID NO: 528) |
| TM-89 | QVQLQQSGAELVRPGTSVKVSCKASGYA | DIQMTQSPSSLSASLGERVSLTCRAS |
| | FTNYLIEWVKQRPGQGLEWIGVINPGSGG | QDIGSSLNWLQQEPDGTIKRLIYATS |
| | TDYNEKFKGKATLTADKSSSTAYMQLSS | SLDSGVPKRFSGSRSGSDYSLTISSL |
| | LTSEDSAVYFCARDPYSKYVHYPMDYW | ESEDFVDYYCLQYASSPRTFGGGTK |
| | GQGTSVTVSS (SEQ ID NO: 494) | LEIK (SEQ ID NO: 529) |
| TM-90 | QVHLQQSGPELVKPGASVKLSCKASGYP | DVLMTQTPLSLPVSLGDQASISCRSS |
| | FTSYDINWVKQRPGQGLEWIGWIYPRDG | QSFVHGNGNTYLEWYLQKPGQSPK |
| | NAKYNEKFKGKATLTVDTSSSTAYMELH | LLIYKVSHRFSGVPDRFSGSGSGTDF |
| | SLTSENSAVYFCARVSDYHGGYGMDYW | TLKITRVEAEDLGVYYCFQNSHVPH |
| | GQGTSVTVSS (SEQ ID NO: 495) | TFGGGTKLEIK (SEQ ID NO: 530) |
| TM-91 | DVQLQESGPGLVKPSQSLSLTCSVTGYSIT | DIVMTQSQKFMSTSVGDRVSVTCK |
| | SGYYWDWIRQFPGNKLEWMGYISYDGN | ASQNVYTNVAWYQQKPGQSPKPLI |
| | NNYNPSLKNRISITRDTSKNQFFLKLNSVT | YSASYRYSGVPDRFTGSGSGTDFTL |
| | TEDTATYYCARAVYSVGFAYWGQGTLV | TISNVQSEDLAEYFCQQYNSYPLTF |
| | TVSS (SEQ ID NO: 496) | GGGTKLEIK (SEQ ID NO: 531) |
| TM-92 | EVQLQQSGPELVKPGASVKIPCKASGYTF | DVQITQSPSYLAASPGETITINCRAS |
| | TDYTMDWVKQSPGKSLEWIGHINPNDGG | KSISKYLAWYQEKPGKTNKLLIYSG |
| | TFYNQKFKGKATLTVDKSSSTAYMELRS | STLQSGIPSRFSGSGSGTDFTLTISSL |
| | LTSEDSAVYYCARWVYYYGISYEGGAM | EPEDFAMYYCQQHNEYPWTFGGGT |
| | DYWGQGTSVTVSS (SEQ ID NO: 497) | KLEIK (SEQ ID NO: 532) |
| TM-93 | QVQLKESGPGLVAPSQSLSITCTVSGFSLS | DIVMTQSHKLMSTSVGDRVSITCKA |
| | SYGISWVRQPPGKGLEWLGVIWTGGGTN | SQDVGSAVAWYQQKPGQSPQLLIY |
| | YNSALESRLSITKDNSKSQVFLKMNSLQT | WSSTRLPGVPDRFTGSGSGTDFTLTI |
| | DDTARYYCARNPPKIFYDYLMYFDYWG | SNVQSEVLADYFCQQYTSYPLTFGG |
| | QGTTVTVSS (SEQ ID NO: 498) | GTKLEIK (SEQ ID NO: 533) |
| TM-94 | EVQLQQSGPELVKPGASVKIPCKASGYTF | DVVMTQTPLSLPVSLGDQASISCRSS |
| | TDYNMDWVKQSHGKSLEWIGDINPYNG | QSLVYSNGNTYLHWYLQRPGQSPK |
| | GTLYNQKFKGKATLTVDKSSNTAYMEIR | LLIYKVSNRFSGVPDRFSGSGSGTDF |
| | SLTYEDTAVYYCARVGYHGTPDYWGQG | TLKISRVEAEDLGVYFCSQSTHVYT |
| | TTVTVSS (SEQ ID NO: 499) | FGGGTKLEIK (SEQ ID NO: 534) |

### Example 3: Anti-viral Activity of Anti-TMEM106B Antibodies

TMEM106B has been identified as a host factor for SARS-CoV-2 coronavirus infection. The anti-viral activity of anti-TMEM106B antibodies toward SARS-CoV-2 coronavirus infectivity and/or replication is determined by various in vitro and in vivo methodologies known and available to one of skill in the art for assessing anti-viral activity of an antibody.

For example, VeroE6 cells (African green monkey kidney epithelial cells) are cultured with anti-TMEM106B antibodies for a period of time, such as one hour. Afterward, the cells are infected with a dilution series of SARS-CoV-2 virus (such as, for example, SARS-CoV-2 coronavirus Washington 2019 virus strain) covering a range of multiplicity of infection (MOD, and the incubated for a period of time (1-3 days, for example). Afterward, cells are fixated and stained with neutral red. Cytotoxicity is evaluated using standard methods. Cell viability is measured using methods known to one of skill in the art, such as, for example, fixation and staining of surviving cells with crystal violet.

Viral cytopathic effect (CPE) is also determined. Intracellular SARS-CoV-2 virus levels in non-fixated cells are determined using RT-qPCR quantification, which further provides a measurement of the effect of anti-TMEM106B antibodies on SARS-CoV-2 viral replication.

Alternatively, other cell types are used in these studies, such as cell lines derived from human liver (e.g., Huh7 cells, Hep3B cells) or cell lines derived from human lung (e.g., A549 cells, NCI-H1975 cells, NCI-H2110 cells).

Results from these in vitro experiments provide an assessment of the anti-viral effect of anti-TMEM106B antibodies on SARS-CoV-2 virus infection and/or SARS-CoV-2 virus replication, including the effect of anti-TMEM106B antibodies on SAR-CoV-2-induced cytopathic effect (CPE).

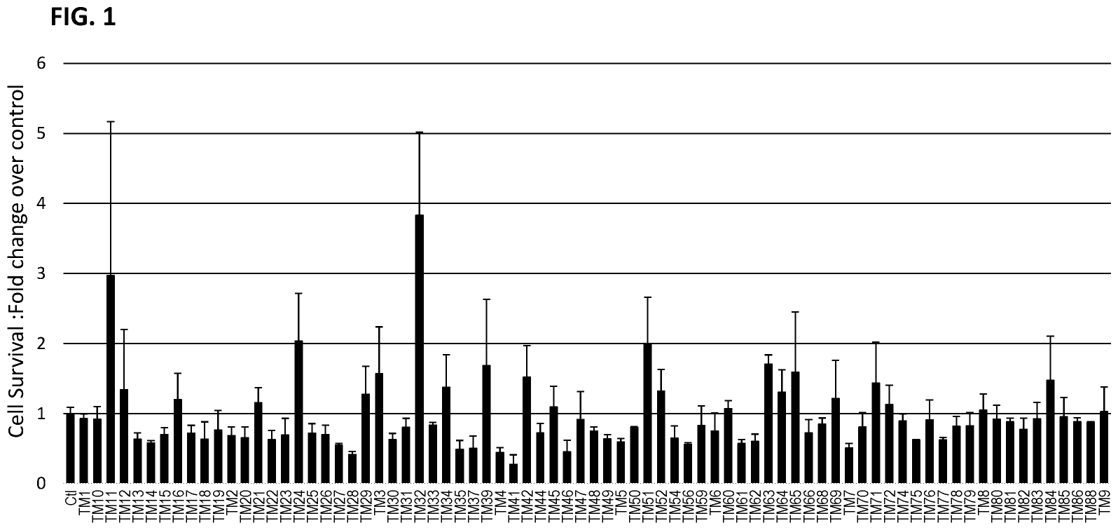

VeroE6 cells were incubated with anti-TMEM106B antibodies of the present disclosure (˜100 μg/ml ) for 1 hour. The cells were then incubated with a dilution series of SARS-CoV-2 coronavirus (Washington 2019 virus strain) as described above. Viral cytopathic effect (CPE), as measured by cell viability/survival, was assessed as described above. The results of these studies are shown in FIG. 1.

Data in FIG. 1 is presented as fold-change in cell survival compared to that observed in control. As shown in FIG. 1, anti-TMEM106B antibodies of the present disclosure were effective at maintaining cell viability following SARS-CoV-2 virus infection, indicating that the anti-TMEM106B antibodies of the present disclosure were effective at reducing viral cytopathic effect. In particular, anti-TMEM106B antibodies TM3, TM11, TM24, TM32, TM34, TM39, TM42, TM51, TM63, TM65, TM71, and TM84 showed approximately 1.5-fold or greater cell survival compared to that observed in virus-infected control cells. Additionally, anti-TMEM106B antibodies TM11, TM24, TM32, and TM51 showed approximately 2-fold or greater cell survival compared to that observed in virus-infected control cells. These results indicated that anti-TMEM106B antibodies of the present disclosure are effective at reducing viral cytopathic effects in cells infected with SARS-CoV-2 coronavirus.

### Example 4: Epitope Binning of anti-TMEM106b Antibodies

Epitope binning of certain anti-TMEM106B antibodies of the present disclosure was performed by Carterra (Salt Lake City, Nevada, USA) using a pre-mix epitope binning approach. Monoclonal anti-TMEM106B antibodies were immobilized to a CMD 50M chip (Xantec # SPMXCMD5OM lot# SCCMD50M0416). The running buffer was HBS-EP+ with 1 mg/mL BSA. The GST-TMEM106B (truncated, comprising amino acids 122-210 of SEQ ID NO:1) antigen was prepared at a final concentration of 55 nM (corresponding to 2 μg/mL) and mixed with the competing analyte anti-TMEM106B antibodies at a final concentration of 333 nM (corresponding to 50 μg/mL) or compared to a buffer control. Samples were injected for 5 minutes over the array and regenerated after every cycle with 1 minute of two parts Pierce IgG-Elution buffer (ThermoFisher Cat#21004) and 1 part of 10 mM Glycine, pH 2.0 (Carterra).

Anti-TMEM106B antibodies sorted into various competing bins and resulting binning profiles. Certain anti-TMEM106B antibodies displayed no ability to block the binding to any of the other anti-TMEM106B antibodies due to their inability to bind the truncated GST-TMEM106B fusion protein used in these binning experiments, indicated that these anti-TMEM106B antibodies bound to the C-terminal domain of TMEM106B; these anti-TMEM106B antibodies were assigned to Bin 5 (see Table 7 below). Other anti-TMEM106B antibodies displayed binning profiles identified as Bin 1 and Bin 2. Bin 2 showed two closely related sub-bins (Bin 3 and Bin 4). Antibodies within Bin 3 or Bin 4 partially block Bin 2 antibodies.

Epitope binning of certain other anti-TMEM106B antibodies of the present disclosure was performed by Lake Pharma (Salt Lake City, Nevada, USA) using a pre-mix epitope binning approach. Monoclonal anti-TMEM106B antibodies were immobilized to a CMD 50M chip (Xantec # SPMXCMD50M lot# SCCMD50M0416). The running buffer was HBS-EP+ with lmg/ml BSA. The GST-TMEM106B (truncated) antigen was prepared at a final concentration of 55 nM (corresponding to 2 μg/ml) and mixed with the competing analyte anti-TMEM106B antibodies at a final concentration of 333 nM (corresponding to 50 μg/ml) or compared to a buffer control. Samples were injected for 5 minutes over the array and regenerated after every cycle with 1 minute of two parts Pierce IgG-Elution buffer (ThermoFisher Cat#21004) and 1 part of 10 mM Glycine, pH 2.0 (Carterra). See Table 7 below.

These results showed that anti-TMEM106B antibodies of the present disclosure bin to different communities (e.g., anti-TMEM106B antibodies that bin to a particular community bind to the same or overlapping epitope), based on the assays used as described above.

### Example 5: Effect of Anti-TMEM106B Antibodies on Cytopathic Effect (CPE) Following SARS-CoV-2 Infection in Vitro

Anti-TMEM106B antibodies of the present disclosure were tested for their ability to affect cell viability and CPE in vitro as follows. NCI-H1975 cells (human lung epithelial cells) were plated in RPMI media containing 8% heat-inactivated FBS in 96-well plates at a cell density of 4×10⁴ cells/ml (100 ill per well). Anti-TMEM106B antibodies were serially diluted (see below for final antibody concentrations) and added to each of the wells; the cells were incubated in the presence of anti-TMEM106B antibodies overnight.

The following day, SARS-CoV-2 virus (SARS-CoV-2 Belgium p6 25-1) was added to the cells; final serially diluted antibody concentrations following virus addition to the wells was 20,000 ng/ml, 2,000 ng/ml, 200 ng/ml, and 20 ng/ml. The cells were then incubated in the presence of anti-TMEM106B antibodies and SARS-CoV-2 virus for three days. Cells were visually observed for cytopathic effect (CPE) induced by the virus and the effect of anti-TMEM106B antibodies thereon. Cell viability was assessed by MTS assay, a colormetric method for determining cell viability. In these studies, the anti-viral compound remdesivir (1 μM) was used as a positive control known to inhibit SARS-CoV-2 induced CPE in NCI-H1975 cells. Hamster anti-SARS-CoV-2 antisera was also used as a positive control in these experiments. These experiments were preformed independently twice.

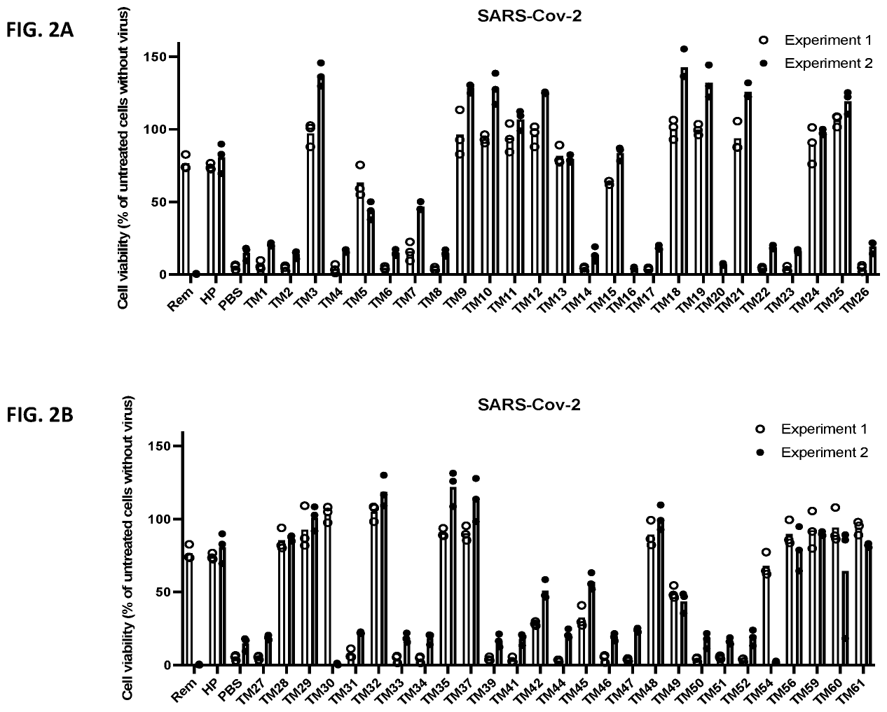

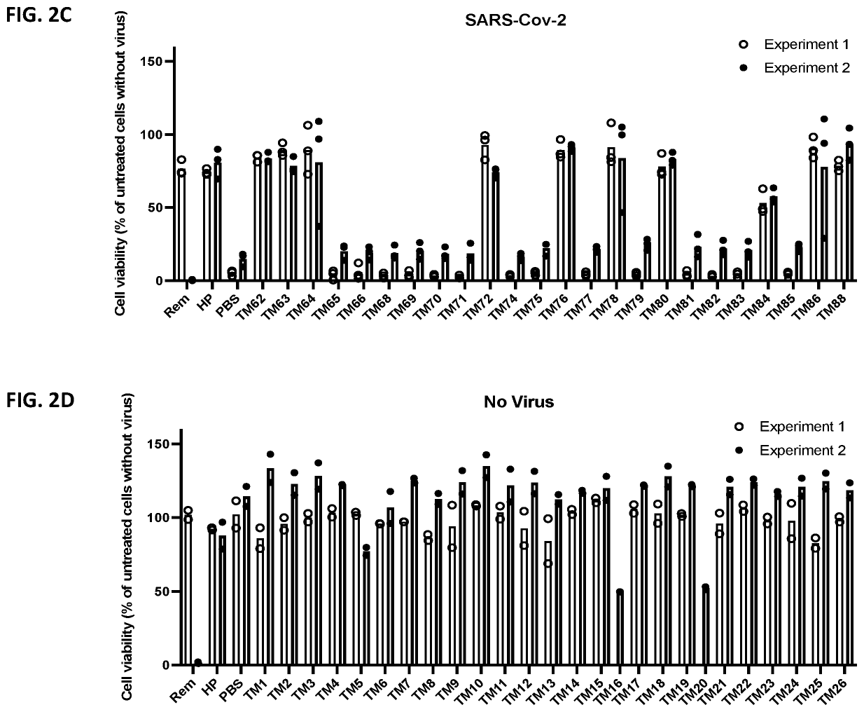

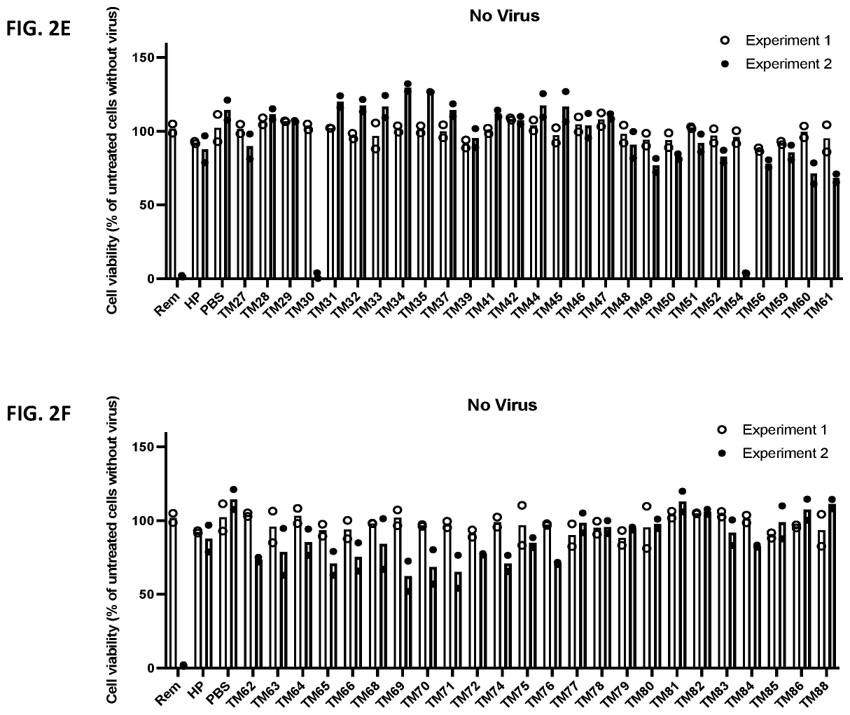

As shown in FIGS. 2A, 2B, 2C, 2D, 2E, and 2F, positive control Remdesivir (Rem) addition to the virus-infected cells resulted in approximately 75-80% cell viability compared to percent cell viability in the absence of virus addition. Additionally, hamster anti-SARS-CoV-2 antisera (HP) addition to the virus-infected cells resulted in approximately 75-80% cell viability compared to percent cell viability in the absence of virus addition, similar to that observed upon Remdesivir treatment. Note that PBS addition in the presence of virus resulted in a significant virus cytopathic effect, as evidenced by less than 20% cell viability.

FIGS. 2A-2C show that anti-TMEM106B antibodies of the present disclosure were at least as effective as Remdesivir at reducing virus cytopathic effect (as measured by cell viability) in SARS-CoV-2 infected NCI-H1975 human lung epithelial cells. In particular, addition of anti-TMEM106B antibodies TM3, TM9, TM10, TM11, TM18, TM19, TM21, TM24, TM25, TM28, TM29, TM30, TM32, TM35, TM37, TM48, TM56, TM59, TM60, TM61, TM63, TM64,TM72, TM76, TM78, TM80, TM86, and TM88 resulted in approximately 80% or greater cell viability comparted to percent cell viability in the absence of virus addition.

### Example 6: Correlation Between Anti-TMEM106B Antibody Epitope Bin and Protection from SARS-CoV-2 Coronavirus Cytopathic Effect

As described above in Example 4, epitope binning analyses showed distinct binning profiles of the anti-TMEM106B antibodies of the present disclosure for binding to TMEM106B polypeptide. Correlation of the effectiveness of anti-TMEM106B antibodies of each of the antibody binning profiles for protection of cell death following coronavirus infection (as determined by CPE) was determined, the results of which are shown below in Table 7. Non-binder, as shown below, indicates an anti-TMEM106B antibody that was not capable of binding to the GST-TMEM106B polypeptide fusion used in these binning experiments (assigned bin 5), which lack the C-terminal portion of TMEM106B.

TABLE 7

| | | % | | | % |
|---|---|---|---|---|---|
| | | Protec- | | | Protec- |
| | Binding | tion | | Binding | tion |
| Anti- | Community/ | from | | Community/ | from |
| body | Bin | CPE | Antibody | Bin | CPE |
| TM25 | 2 | 106.3 | TM45 | 2 | 32.6 |
| TM32 | 2 | 104.8 | TM42 | 2 | 28.4 |
| TM30 | 4 | 103.5 | TM7 | 3 | 15.8 |
| TM18 | 2 | 100.4 | TM31 | Non-binder | 7.7 |
| TM19 | 2 | 99.8 | TM1 | 1 | 6.4 |
| TM3 | 2 | 97.2 | TM66 | Non-binder | 5.9 |
| TM9 | 2 | 96.4 | TM51 | Non-binder | 5.4 |
| TM12 | 2 | 95.9 | TM27 | Non-binder | 5.2 |
| TM60 | 2 | 94.2 | TM85 | Non-binder | 5.2 |
| TM61 | 2 | 94.1 | TM75 | Non-binder | 5.1 |
| TM11 | 2 | 93.9 | TM46 | Non-binder | 4.7 |
| TM21 | 2 | 93.7 | TM77 | Non-binder | 4.7 |
| TM10 | 2 | 93.3 | TM81 | Non-binder | 4.6 |
| TM72 | 4 | 92.7 | TM79 | 1 | 4.5 |
| TM29 | 4 | 92.6 | TM6 | Non-binder | 4.4 |
| TM59 | 2 | 92.3 | TM22 | 1 | 4.4 |
| TM78 | 4 | 91.1 | TM33 | Non-binder | 4.4 |
| TM86 | 4 | 90.5 | TM2 | Non-binder | 4.3 |
| TM35 | 2 | 90.4 | TM26 | 1 | 4.3 |
| TM37 | 2 | 90.2 | TM69 | Non-binder | 4.3 |
| TM56 | Non-binder | 89.7 | TM83 | 3 | 4.3 |
| TM24 | 2 | 8.4 | TM39 | Non-binder | 4.2 |
| TM48 | 2 | 89.3 | TM34 | Non-binder | 4.1 |
| TM63 | 4 | 89.3 | TM14 | Non-binder | 4.0 |
| TM64 | 4 | 89.3 | TM65 | Non-binder | 4.0 |
| TM76 | 2 | 89.2 | TM50 | Non-binder | 3.9 |
| TM28 | 4 | 85.4 | TM8 | Non-binder | 3.8 |
| TM62 | Non-binder | 84.2 | TM47 | Non-binder | 3.8 |
| TM13 | 2 | 81.8 | TM4 | Non-binder | 3.7 |
| TM88 | 4 | 78.5 | TM17 | 1 | 3.7 |
| TM80 | 4 | 78 | TM23 | Not determined | 3.7 |
| TM54 | 2 | 68.1 | TM74 | Non determined | 3.7 |
| TM15 | 3 | 63.5 | TM52 | Non-binder | 3.6 |
| TM5 | 4 | 63.3 | TM41 | Non-binder | 3.5 |
| TM84 | 3 | 53.1 | TM68 | Not determined | 3.5 |
| TM49 | Non-binder | 49.6 | TM70 | Non-binder | 3.4 |
| | | | TM44 | Non-binder | 3.2 |
| | | | TM71 | Non-binder | 3.2 |
| | | | TM82 | 1 | 3.2 |

As shown in Table 7, anti-TMEM106B antibodies that belong to bin 2 (and related sub-bins 3 and 4) were effective at preventing virus cytopathic effect (as measured by cell death), whereas anti-TMEM106B antibodies belonging to bin 1 or bin 5 were not. In particular, anti-TMEM106B antibodies belonging to bins 2, 3 and 4 provided greater than 50% protection against cytopathic effect associated with coronavirus infection. In particular, the majority of anti-TMEM106B antibodies belonging to bins 2, 3, and 4 showed greater than 80% protection against cytopathic effect upon coronavirus infection (TM-25, TM-32, TM30, TM18, TM19, TM3, TM9, TM12, TM60, TM61, TM11, TM21, TM10, TM72, TM29, TM59, TM78, TM86, TM35, TM37, TM56, TM24, TM48, TM63, TM64, TM76, TM28, TM72, and TM13). Taken together, these results suggested that anti-TMEM106B antibodies that display a binning profile that overlaps with bins 2, 3, and 4 (as shown above) are likely binding to an epitope of TMEM106B polypeptide that prevents or reduces coronavirus infection.

### Example 7: Dose Titration of Anti-TMEM106B Antibodies on SARS-CoV-2 Infection

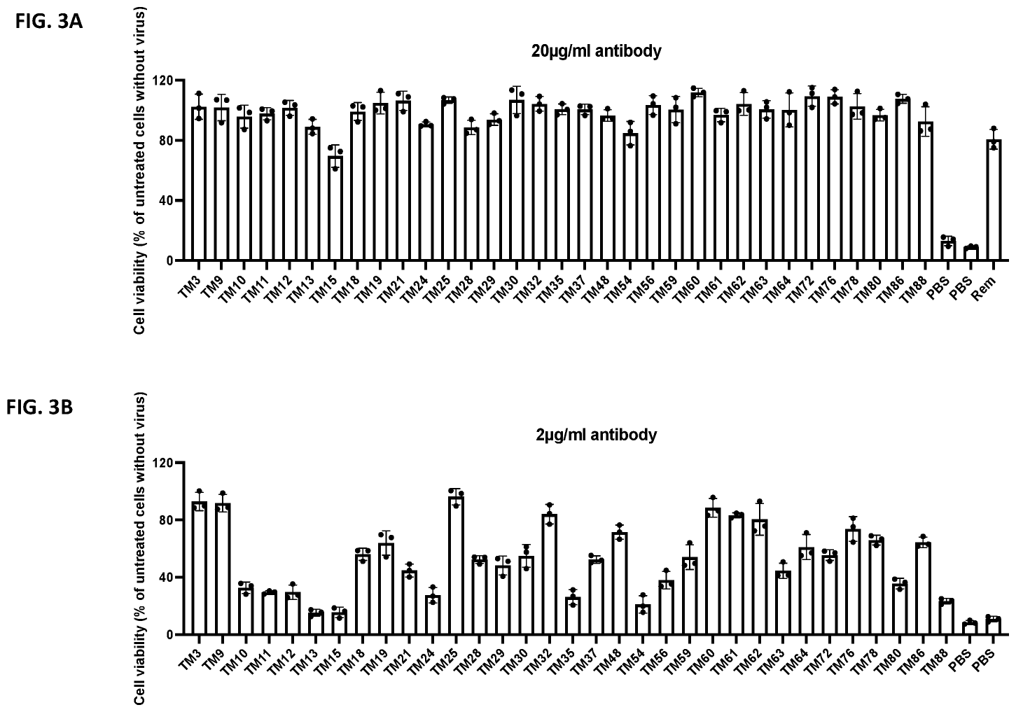

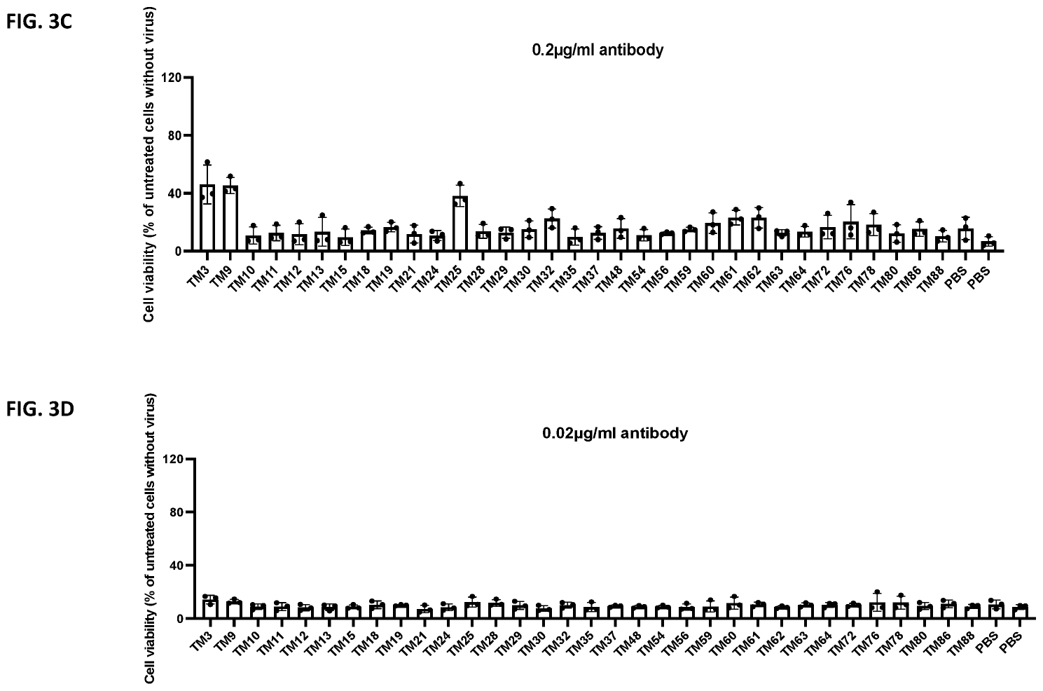

NCI-H1975 cells were treated with four different concentrations of anti-TMEM106B antibodies of the present disclosure (20 μg/mL, 2 μg/mL, 0.2 μg/mL, and 0.02 μg/mL) or remdesivir overnight before being infected with SARS-CoV-2 virus for 3 days. At the end of the experiment, cytopathic effects were assessed visually, and cell viability was measured by MTS assay. Cell viability is normalized to cell viability observed in untreated/uninfected cells is shown in FIGS. 3A-D. As shown in FIGS. 3A-D, certain anti-TMEM106B antibodies of the present disclosure were effective reducing cytopathic effects associated with SARS-CoV-2 infection in vitro at concentrations as low as 200 ng/mL.

What is claimed is:

1. A pharmaceutical composition for use in treating, preventing, or reducing the risk of coronavirus infection comprising an antibody that binds to a TMEM106B protein and a pharmaceutically acceptable carrier.

2. An antibody that binds to a TMEM106B protein for use in treating, preventing, or reducing the risk of coronavirus infection.

3. A method for treating, preventing, or reducing the risk of coronavirus infection, the method comprising administering to an individual in need thereof an effective amount of an antibody that binds to a TMEM106B protein.

4. The pharmaceutical composition, antibody, or method of any one of claims 1-3, wherein the antibody reduces coronavirus replication.

5. The pharmaceutical composition, antibody, or method of any one of claims 1-4, wherein the antibody reduces coronavirus transmission.

6. The pharmaceutical composition, antibody, or method of any one of claims 1-5, wherein the antibody reduces coronavirus genome translation.

7. The pharmaceutical composition, antibody, or method of any one of claims 1-6, wherein the antibody reduces coronavirus cell entry.

8. The pharmaceutical composition, antibody, or method of any one of claims 1-7, wherein the antibody reduces coronavirus release from an infected cell.

9. The pharmaceutical composition, antibody, or method of any one of claims 1-8, wherein coronavirus is SARS-CoV-2 coronavirus.

10. The pharmaceutical composition, antibody, or method of any one of claims 1-9, wherein the TMEM106B protein is a mammalian protein or a human protein.

11. The pharmaceutical composition, antibody, or method of any one of claims 1-10, wherein the antibody binds to the cytoplasmic domain of TMEM106B protein.

12. The pharmaceutical composition, antibody, or method of any one of claims 1-10, wherein the antibody binds to the luminal domain of TMEM106B protein.

13. The pharmaceutical composition, antibody, or method of any one of claims 1-12, wherein the antibody comprises at least one, two, three, four, five, or six HVRs of an antibody selected from the group consisting of TM-1, TM-2, TM-3, TM-4, TM-5, TM-6, TM-7, TM-8, TM-9, TM-10, TM-11, TM-12, TM-13, TM-14, TM-15, TM-16, TM-17, TM-18, TM-19, TM-20, TM-21, TM-22, TM-23, TM-24, TM-25, TM-26, TM-27, TM-28, TM-29, TM-30, TM-31, TM-32, TM-33, TM-34, TM-35, TM-37, TM-39, TM-41, TM-42, TM-43, TM-44, TM-45, TM-46, TM-47, TM-48, TM-49, TM-50, TM-51, TM-52, TM-54, TM-56, TM-59, TM-60, TM-61, TM-62, TM-63, TM-64, TM-65, TM-66, TM-67, TM-68, TM-69, TM-70, TM-71, TM-72, TM-73, TM-74, TM-75, TM-76, TM-77, TM-78, TM-79, TM-80. TM-81, TM-82, TM-83, TM-84, TM-85, TM-86, TM-87, TM-88, TM-89, TM90, TM-91, TM-92, TM-93, and TM-94, optionally wherein the HVRs are Kabat, AbM, Chothia, or Contact HVRs.

14. The pharmaceutical composition, antibody or method of claim 13, wherein the antibody comprises a heavy chain variable region comprising an HVR-H1, an HVR-H2, and an HVR-H3 and a light chain variable region comprising an HVR-L1, an HVR-L2, and an HVR-L3, wherein the HVR-H1, HVR-H2, HVR-H3, HVR-L1, HVR-L2, and HVR-L3 comprise the amino acid sequences of
(a) SEQ ID NOs:2, 35, 78, 120, 160, and 184, respectively;
(b) SEQ ID NOs:3, 36, 79, 121, 161, and 185, respectively;
(c) SEQ ID NOs:4, 37, 80, 122, 162, and 186, respectively;
(d) SEQ ID NOs:5, 38, 81, 123, 163, and 187, respectively;
(e) SEQ ID NOs:6, 39, 82, 124, 164, and 188, respectively;
(f) SEQ ID NOs:7, 40, 83, 125, 165, and 189, respectively;
(g) SEQ ID NOs:8, 41, 84, 126, 166, and 190, respectively;
(h) SEQ ID NOs:9, 42, 85, 127, 167, and 191, respectively;
(i) SEQ ID NOs:10, 43, 86, 122, 168, and 186, respectively;
(j) SEQ ID NOs:11, 44, 87, 128, 169, and 192, respectively;
(k) SEQ ID NOs:12, 45, 88, 129, 170, and 193, respectively;
(1) SEQ ID NOs:13, 46, 89, 130, 169, and 194, respectively;
(m) SEQ ID NOs:14, 47, 90, 131, 171, and 195, respectively;
(n) SEQ ID NOs:2, 48, 91, 132, 172, and 196, respectively;
(o) SEQ ID NOs:15, 49, 92, 133, 171, and 197, respectively;
(p) SEQ ID NOs:16, 50, 93, 134, 173, and 198, respectively;
(q) SEQ ID NOs:17, 51, 94, 132, 172, and 196, respectively;
(r) SEQ ID NOs:5, 52, 95, 135, 174, and 199, respectively;
(s) SEQ ID NOs:13, 53, 96, 136, 175, and 200, respectively;
(t) SEQ ID NOs:16, 50, 93, 134, 173, and 198, respectively;
(u) SEQ ID NOs:18, 54, 97, 137, 168, and 201, respectively;
(v) SEQ ID NOs:19, 55, 98, 138, 160, and 202, respectively;
(w) SEQ ID NOs:2, 56, 99, 139, 176, and 203, respectively;
(x) SEQ ID NOs:12, 45, 88, 140, 170, and 204, respectively;
(y) SEQ ID NOs:10, 57, 100, 137, 168, and 205, respectively;
(z) SEQ ID NOs:2, 56, 99, 139, 176, and 203, respectively;
(aa) SEQ ID NOs:2, 58, 101, 141, 177, and 206, respectively;
(bb) SEQ ID NOs:8, 59, 102, 142, 166, and 190, respectively;
(cc) SEQ ID NOs:20, 60, 103, 143, 168, and 207, respectively;
(dd) SEQ ID NOs:21, 61, 104, 144, 160, and 208, respectively;
(ee) SEQ ID NOs:11, 62, 105, 127, 178, and 209, respectively;
(ff) SEQ ID NOs:13, 63, 106, 145, 160, and 200, respectively;
(gg) SEQ ID NOs:22, 64, 107, 146, 179, and 210, respectively;
(hh) SEQ ID NOs:23, 65, 108, 147, 161, and 211, respectively;
(ii) SEQ ID NOs:24, 66, 109, 148, 160, and 194, respectively;
(jj) SEQ ID NOs:25, 67, 110, 149, 180, and 212, respectively;
(kk) SEQ ID NOs:26, 68, 111, 150, 180, and 213, respectively;
(ll) SEQ ID NOs:27, 69, 112, 151, 181, and 214, respectively;
(mm) SEQ ID NOs:28, 70, 110, 152, 180, and 212, respectively;
(nn) SEQ ID NOs:29, 71, 113, 151, 181, and 215, respectively;
(oo) SEQ ID NOs:28, 70, 110, 152, 180, and 212, respectively;
(pp) SEQ ID NOs:30, 72, 114, 153, 182, and 216, respectively;
(qq) SEQ ID NOs:31, 73, 115, 154, 181, and 217, respectively;
(rr) SEQ ID NOs:32, 74, 116, 155, 160, and 218, respectively;
(ss) SEQ ID NOs:33, 75, 117, 156, 182, and 219, respectively;
(tt) SEQ ID NOs:31, 73, 115, 157, 183, and 220, respectively;
(uu) SEQ ID NOs:34, 76, 118, 158, 181, and 221, respectively;
(vv) SEQ ID NOs:28, 77, 119, 159, 182, and 222, respectively;
(ww) SEQ ID NOs:223, 244, 277, 155, 160, and 202, respectively;
(xx) SEQ ID NOs:25, 245, 110, 307, 180, and 212, respectively;
(yy) SEQ ID NOs:25, 67, 110, 149, 180, and 212, respectively;
(zz) SEQ ID NOs:32, 74, 116, 155, 160, and 218, respectively;
(aaa) SEQ ID NOs:224, 246, 278, 142, 166, and 349, respectively;
(bbb) SEQ ID NOs:225, 247, 279, 156, 182, and 219, respectively;
(ccc) SEQ ID NOs:226, 248, 280, 308, 180, and 350, respectively;
(ddd) SEQ ID NOs:25, 249, 281, 309, 180, and 351, respectively;
(eee) SEQ ID NOs:227, 250, 282, 310, 335, and 352, respectively;
(fff) SEQ ID NOs:18, 251, 283, 311, 176, and 353, respectively;
(ggg) SEQ ID NOs:228, 252, 284, 312, 180, and 354, respectively;
(hhh) SEQ ID NOs:229, 253, 285, 313, 160, and 355, respectively;
(iii) SEQ ID NOs:18, 254, 286, 314, 336, and 356, respectively;
(jjj) SEQ ID NOs:18, 254, 286, 309, 337, and 351, respectively;
(kkk) SEQ ID NOs:28, 255, 287, 309, 337, and 351, respectively;
(lll) SEQ ID NOs:230, 256, 288, 315, 173, and 357, respectively;
(mmm) SEQ ID NOs:231, 257, 289, 316, 338, and 358, respectively;
(nnn) SEQ ID NOs:232, 258, 290, 317, 339, and 359, respectively;
(ooo) SEQ ID NOs:233, 259, 291, 318, 340, and 360, respectively;
(ppp) SEQ ID NOs:234, 260, 292, 319, 341, and 361, respectively;
(qqq) SEQ ID NOs:235, 261, 287, 320, 180, and 351, respectively;
(rrr) SEQ ID NOs:236, 262, 293, 321, 342, and 362, respectively;
(sss) SEQ ID NOs:237, 263, 280, 322, 180, and 363, respectively;
(ttt) SEQ ID NOs:25, 264, 294, 323, 343, and 204, respectively;
(uuu) SEQ ID NOs:2, 265, 295, 324, 176, and 364, respectively;
(vvv) SEQ ID NOs:238, 266, 296, 325, 160, and 365, respectively;
(www) SEQ ID NOs:14, 47, 90, 131, 171, and 366, respectively;
(xxx) SEQ ID NOs:239, 267, 297, 326, 344, and 367, respectively;
(yyy) SEQ ID NOs:8, 268, 298, 142, 166, and 190, respectively;
(zzz) SEQ ID NOs:5, 269, 299, 327, 345, and 368, respectively;
(aaaa) SEQ ID NOs:20, 270, 300, 328, 346, and 369, respectively;
(bbbb) SEQ ID NOs:2, 271, 301, 329, 172, and 196, respectively;
(cccc) SEQ ID NOs:32, 272, 302, 330, 347, and 370, respectively;
(dddd) SEQ ID NOs:240, 273, 303, 331, 344, and 367, respectively;
(eeee) SEQ ID NOs:241, 274, 304, 332, 345, and 371, respectively;
(ffff) SEQ ID NOs:242, 275, 305, 333, 348, and 372, respectively; or
(gggg) SEQ ID NOs:243, 276, 306, 334, 160, and 373, respectively.

15. The pharmaceutical composition, antibody or method of claim 14, wherein the heavy chain variable region and the light chain variable region comprise the amino acid sequences of
(a) SEQ ID NOs:374 and 418, respectively;
(b) SEQ ID NOs:375 and 419, respectively;
(c) SEQ ID NOs:376 and 420, respectively;
(d) SEQ ID NOs: 377 and 421, respectively;
(e) SEQ ID NOs:378 and 422, respectively;
(f) SEQ ID NOs:379 and 423, respectively;
(g) SEQ ID NOs:380 and 424, respectively;
(h) SEQ ID NOs:381 and 425, respectively;
(i) SEQ ID NOs:382 and 426, respectively;
(j) SEQ ID NOs:383 and 427, respectively;
(k) SEQ ID NOs:384 and 428, respectively;
(l) SEQ ID NOs:385 and 429, respectively;
(m) SEQ ID NOs:386 and 430, respectively;
(n) SEQ ID NOs:387 and 431, respectively;
(o) SEQ ID NOs:388 and 432, respectively;
(p) SEQ ID NOs:389 and 433, respectively;
(q) SEQ ID NOs:390 and 434, respectively;
(r) SEQ ID NOs:391 and 435, respectively;
(s) SEQ ID NOs:392 and 436, respectively;
(t) SEQ ID NOs:389 and 437, respectively;
(u) SEQ ID NOs:393 and 438, respectively;
(v) SEQ ID NOs:394 and 439, respectively;
(w) SEQ ID NOs:395 and 440, respectively;
(x) SEQ ID NOs:384 and 441, respectively;
(y) SEQ ID NOs:396 and 442, respectively;
(z) SEQ ID NOs:395 and 440, respectively;
(aa) SEQ ID NOs:397 and 443, respectively;
(bb) SEQ ID NOs:398 and 444, respectively;
(cc) SEQ ID NOs:399 and 445, respectively;
(dd) SEQ ID NOs:400 and 446, respectively;
(ee) SEQ ID NOs:401 and 447, respectively;
(ff) SEQ ID NOs:402 and 448, respectively;
(gg) SEQ ID NOs:403 and 449, respectively;
(hh) SEQ ID NOs:404 and 450, respectively;
(ii) SEQ ID NOs:405 and 451, respectively;
(jj) SEQ ID NOs:406 and 452, respectively;
(kk) SEQ ID NOs:407 and 453, respectively;
(ll) SEQ ID NOs:408 and 454, respectively;
(mm) SEQ ID NOs:409 and 455, respectively;
(nn) SEQ ID NOs:410 and 456, respectively;
(oo) SEQ ID NOs:409 and 455, respectively;
(pp) SEQ ID NOs:411 and 457, respectively;
(qq) SEQ ID NOs:412 and 458, respectively;
(rr) SEQ ID NOs:413 and 459, respectively;
(ss) SEQ ID NOs:414 and 460, respectively;
(tt) SEQ ID NOs:415 and 461, respectively;
(uu) SEQ ID NOs:416 and 462, respectively;
(vv) SEQ ID NOs:417 and 463, respectively;
(ww) SEQ ID NOs:464 and 500, respectively;
(xx) SEQ ID NOs:465 and 501, respectively;
(yy) SEQ ID NOs:466 and 452, respectively;
(zz) SEQ ID NOs:413 and 502, respectively;
(aaa) SEQ ID NOs:467 and 503, respectively;
(bbb) SEQ ID NOs:468 and 504, respectively;
(ccc) SEQ ID NOs:469 and 505, respectively;
(ddd) SEQ ID NOs:470 and 506, respectively;
(eee) SEQ ID NOs:471 and 507, respectively;
(fff) SEQ ID NOs:472 and 508, respectively;
(ggg) SEQ ID NOs:473 and 509, respectively;
(hhh) SEQ ID NOs:474 and 510, respectively;
(iii) SEQ ID NOs:475 and 511, respectively;
(jjj) SEQ ID NOs:476 and 512, respectively;
(kkk) SEQ ID NOs:477 and 512, respectively;
(111) SEQ ID NOs:478 and 513, respectively;
(mmm) SEQ ID NOs:479 and 514, respectively;
(nnn) SEQ ID NOs:480 and 515, respectively;
(ooo) SEQ ID NOs:481 and 516, respectively;
(ppp) SEQ ID NOs:482 and 517, respectively;
(qqq) SEQ ID NOs:483 and 518, respectively;
(rrr) SEQ ID NOs:484 and 519, respectively;
(sss) SEQ ID NOs:485 and 520, respectively;
(ttt) SEQ ID NOs:486 and 521, respectively;
(uuu) SEQ ID NOs:487 and 522, respectively;
(vvv) SEQ ID NOs:488 and 523, respectively;
(www) SEQ ID NOs:489 and 524, respectively;
(xxx) SEQ ID NOs:490 and 525, respectively;
(yyy) SEQ ID NOs:491 and 526, respectively;
(zzz) SEQ ID NOs:492 and 527, respectively;
(aaaa) SEQ ID NOs:493 and 528, respectively;
(bbbb) SEQ ID NOs:494 and 529, respectively;
(cccc) SEQ ID NOs:495 and 530, respectively;
(dddd) SEQ ID NOs:496 and 531, respectively;
(eeee) SEQ ID NOs:497 and 532, respectively;
(ffff) SEQ ID NOs:498 and 533, respectively; or
(gggg) SEQ ID NOs:499 and 534, respectively.

16. The pharmaceutical composition, antibody or method of any one of claims 1-14, wherein the antibody competitively inhibits the binding to TMEM106B of an antibody comprising the heavy chain variable region and the light chain variable region of any one of the antibodies in subparts (a)-(gggg) of claim 15.

17. The pharmaceutical composition, antibody or method any one of claims 1-16, wherein the antibody binds a truncated TMEM106B protein comprising amino acids 122-210 of SEQ ID NO:1.

18. The pharmaceutical composition, antibody or method of any one of claims 1-17, wherein the antibody is in Bin 2.

19. The pharmaceutical composition, antibody or method of any one of claims 1-17, wherein the antibody is in Bin 3.

20. The pharmaceutical composition, antibody or method of any one of claims 1-17, wherein the antibody is in Bin 4.

21. The pharmaceutical composition of any one of claims 1-20, wherein the antibody is not in Bin 1.

22. The pharmaceutical composition, antibody or method of any one of claims 1-21, wherein the antibody is not in Bin 5.

23. The pharmaceutical composition, antibody or method of any one of claims 1-22, wherein the antibody reduces a cytopathic effect in a cell infected with SARS-CoV-2, optionally wherein the cell is a VeroE6 cell or a NCI-H1975 cell.

24. The pharmaceutical composition, antibody, or method of any one of claims 1-23, wherein the antibody is a monoclonal antibody.

25. The pharmaceutical composition, antibody, or method of any one of claims 1-24, wherein the antibody is of the IgG class, the IgM class, or the IgA class.

26. The pharmaceutical composition, antibody, or method of claim 25, wherein the antibody is of the IgG class and has an IgG1, IgG2, or IgG4 isotype.

27. The pharmaceutical composition, antibody, or method of any one of claims 1-26, wherein the antibody is an antibody fragment.

28. The pharmaceutical composition, antibody, or method of claim 27, wherein the fragment is a Fab, Fab′, Fab′-SH, F(ab′)2, FIT or scFv fragment.

29. The pharmaceutical composition, antibody, or method of any one of claims 1-26, wherein the antibody is a full-length antibody.


SEQUENCE LISTING (534 sequences)

SEQ ID NO: 1 - protein, 274 aa, Artificial Sequence
  Human TMEM106B
MGKSLSHLPLHSSKEDAYDGVTSENMRNGLVNSEVHNEDGRNGDVSQFPYVEFTGRDSVTCPTCQGTGRIPRGQENQLVALIPYSDQRLRPRRTKLYVMASVFVCLLLSGLAVFFLFPRSIDVKYIGVKSAYVSYDVQKRTIYLNITNTLNITNNNYYSVEVENITAQVQFSKTVIGKARLNNITIIGPLDMKQIDYTVPTVIAEEMSYMYDFCTLISIKVHNIVLMMQVTVTTTYFGHSEQISQERYQYVDCGRNTTYQLGQSEYLNVLQPQQ

SEQ ID NO: 2 - protein, 5 aa, Artificial Sequence
  CDR-H1 -- TM-1, TM-14, TM-23, TM-26, TM-27, TM-82, TM-89
NYLIE

SEQ ID NO: 3 - protein, 5 aa, Artificial Sequence
  CRD-H1 -- TM-2
EYPMH

SEQ ID NO: 4 - protein, 7 aa, Artificial Sequence
  CDR-H1 -- TM-3
TLGRGVG

SEQ ID NO: 5 - protein, 5 aa, Artificial Sequence
  CDR-H1 -- TM-4, TM-18, TM-87
DYYMN

SEQ ID NO: 6 - protein, 5 aa, Artificial Sequence
  CDR-H1 -- TM-5
TYGIT

SEQ ID NO: 7 - protein, 5 aa, Artificial Sequence
  CDR-H1 -- TM-6
EYTIH

SEQ ID NO: 8 - protein, 5 aa, Artificial Sequence
  CDR-H1 -- TM-7, TM-28, TM-86
DYYIH

SEQ ID NO: 9 - protein, 5 aa, Artificial Sequence
  CDR-H1 -- TM-8
DYPMH

SEQ ID NO: 10 - protein, 5 aa, Artificial Sequence
  CDR-H1 -- TM-9, TM-25
GYGMS

SEQ ID NO: 11 - protein, 5 aa, Artificial Sequence
  CDR-H1 -- TM-10, TM-31
SYWMN

SEQ ID NO: 12 - protein, 5 aa, Artificial Sequence
  CDR-H1 -- TM-11, TM-24
DYGVH

SEQ ID NO: 13 - protein, 5 aa, Artificial Sequence
  CDR-H1 -- TM-12, TM-19, TM-32
DYYMY

SEQ ID NO: 14 - protein, 5 aa, Artificial Sequence
  CDR-H1 -- TM-13, TM-84
TFPIE

SEQ ID NO: 15 - protein, 5 aa, Artificial Sequence
  CDR-H1 -- TM-15
NYWIT

SEQ ID NO: 16 - protein, 5 aa, Artificial Sequence
  CDR-H1 -- TM-16, TM-20
DYYMH

SEQ ID NO: 17 - protein, 5 aa, Artificial Sequence
  CDR-H1 -- TM-17
DYLIE

SEQ ID NO: 18 - protein, 5 aa, Artificial Sequence
  CDR-H1 -- TM-21, TM-66, TM-70, TM-71
NYAMS

SEQ ID NO: 19 - protein, 5 aa, Artificial Sequence
  CDR-H1 -- TM-22
SYYIH

SEQ ID NO: 20 - protein, 5 aa, Artificial Sequence
  CDR-H1 -- TM-29, TM-88
DYYIN

SEQ ID NO: 21 - protein, 5 aa, Artificial Sequence
  CDR-H1 -- TM-30
NYDVN

SEQ ID NO: 22 - protein, 5 aa, Artificial Sequence
  CDR-H1 -- TM-33
DYNMN

SEQ ID NO: 23 - protein, 5 aa, Artificial Sequence
  CDR-H1 -- TM-34
DSGMD

SEQ ID NO: 24 - protein, 5 aa, Artificial Sequence
  CDR-H1 -- TM-35
RHWMQ

SEQ ID NO: 25 - protein, 5 aa, Artificial Sequence
  CDR-H1 -- TM-37, TM-56, TM-59, TM-64, TM-81
SYWMH

SEQ ID NO: 26 - protein, 5 aa, Artificial Sequence
  CDR-H1 -- TM-39
DYWMH

SEQ ID NO: 27 - protein, 5 aa, Artificial Sequence
  CDR-H1 -- TM-41
DCYMH

SEQ ID NO: 28 - protein, 5 aa, Artificial Sequence
  CDR-H1 -- TM-42, TM-45, TM-52, TM-72
NYWMH

SEQ ID NO: 29 - protein, 5 aa, Artificial Sequence
  CDR-H1 -- TM-44
DYGMH

SEQ ID NO: 30 - protein, 5 aa, Artificial Sequence
  CDR-H1 -- TM-46
SFWMN

SEQ ID NO: 31 - protein, 5 aa, Artificial Sequence
  CDR-H1 -- TM-47, TM-50
NYGVH

SEQ ID NO: 32 - protein, 5 aa, Artificial Sequence
  CDR-H1 -- TM-48, TM-60, TM-90
SYDIN

SEQ ID NO: 33 - protein, 5 aa, Artificial Sequence
  CDR-H1 -- TM-49
RFWMH

SEQ ID NO: 34 - protein, 5 aa, Artificial Sequence
  CDR-H1 -- TM-51
TYWID

SEQ ID NO: 35 - protein, 17 aa, Artificial Sequence
  CDR-H2 -- TM-1
VINPGSGGTKYNEKLKG

SEQ ID NO: 36 - protein, 17 aa, Artificial Sequence
  CDR-H2 -- TM-2
MIYTNTGEPTYAAEFKG

SEQ ID NO: 37 - protein, 16 aa, Artificial Sequence
  CDR-H2 -- TM-3
KIWWNDDKFYYPALKS

SEQ ID NO: 38 - protein, 17 aa, Artificial Sequence
  CDR-H2 -- TM-4
VINPYNGGTSYHQKFKG

SEQ ID NO: 39 - protein, 17 aa, Artificial Sequence
  CDR-H2 -- TM-5
EIYPRSDNTYYNEKFKD

SEQ ID NO: 40 - protein, 17 aa, Artificial Sequence
  CDR-H2 -- TM-6
WFYPGSTYIDYNEKFKD

SEQ ID NO: 41 - protein, 17 aa, Artificial Sequence
  CDR-H2 -- TM-7
LVYPYNGDTDYNQKFKG

SEQ ID NO: 42 - protein, 17 aa, Artificial Sequence
  CDR-H2 -- TM-8
VIYTDTGEPKYAEVFKG

SEQ ID NO: 43 - protein, 17 aa, Artificial Sequence
  CDR-H2 -- TM-9
TISSGSFYIYYPDSVKG

SEQ ID NO: 44 - protein, 17 aa, Artificial Sequence
  CDR-H2 -- TM-10
QIYPGDGDTNYNGKFKG

SEQ ID NO: 45 - protein, 16 aa, Artificial Sequence
  CDR-H2 -- TM-11, TM-24
VIWNNGNTDYNAAFIS

SEQ ID NO: 46 - protein, 17 aa, Artificial Sequence
  CDR-H2 -- TM-12
RIDPEDGDAEYAPKFQG

SEQ ID NO: 47 - protein, 17 aa, Artificial Sequence
  CDR-H2 -- TM-13, TM-84
NFHPYNDDTKYNEKFKG

SEQ ID NO: 48 - protein, 17 aa, Artificial Sequence
  CDR-H2 -- TM-14
VINPGGGNTDYSEKFKD

SEQ ID NO: 49 - protein, 17 aa, Artificial Sequence
  CDR-H2 -- TM-15
DIYPGSGNSNYNESFKR

SEQ ID NO: 50 - protein, 17 aa, Artificial Sequence
  CDR-H2 -- TM-16, TM-20
RIDPEDGETKYAPEFQG

SEQ ID NO: 51 - protein, 17 aa, Artificial Sequence
  CDR-H2 -- TM-17
VINPGSGGTNYNEKFKG

SEQ ID NO: 52 - protein, 17 aa, Artificial Sequence
  CDR-H2 -- TM-18
NINPNNGDAFYNQKFKG

SEQ ID NO: 53 - protein, 17 aa, Artificial Sequence
  CDR-H2 -- TM-19
RIDPEDGDTENAPKFRG

SEQ ID NO: 54 - protein, 17 aa, Artificial Sequence
  CDR-H2 -- TM-21
FISDGGGYIYYADNVKD

SEQ ID NO: 55 - protein, 17 aa, Artificial Sequence
  CDR-H2 -- TM-22
WIYPGNGITNYNEKFKG

SEQ ID NO: 56 - protein, 17 aa, Artificial Sequence
  CDR-H2 -- TM-23, TM-26
VINPGSGITNYNEKFKG

SEQ ID NO: 57 - protein, 17 aa, Artificial Sequence
  CDR-H2 -- TM-25
TISSGGRYTVYPDSVKG

SEQ ID NO: 58 - protein, 17 aa, Artificial Sequence
  CDR-H2 -- TM-27
VINPGSGSTKYNEKFKG

SEQ ID NO: 59 - protein, 17 aa, Artificial Sequence
  CDR-H2 -- TM-28
LVYPYNGGTNYNQNFKG

SEQ ID NO: 60 - protein, 17 aa, Artificial Sequence
  CDR-H2 -- TM-29
RIYPGSGYTYYNEKFKG

SEQ ID NO: 61 - protein, 17 aa, Artificial Sequence
  CDR-H2 -- TM-30
WIYPRDGTTIYNEKFKG

SEQ ID NO: 62 - protein, 17 aa, Artificial Sequence
  CDR-H2 -- TM-31
YINPTSGYTRYNQKFKD

SEQ ID NO: 63 - protein, 17 aa, Artificial Sequence
  CDR-H2 -- TM-32
RIDPEDGDTEYVPKFQG

SEQ ID NO: 64 - protein, 17 aa, Artificial Sequence
  CDR-H2 -- TM-33
VINPNYGTTSYNQKFKG

SEQ ID NO: 65 - protein, 17 aa, Artificial Sequence
  CDR-H2 -- TM-34
YISSGSSTTHYADTVKG

SEQ ID NO: 66 - protein, 17 aa, Artificial Sequence
  CDR-H2 -- TM-35
EILPGSNNIYYNEKVKG

SEQ ID NO: 67 - protein, 17 aa, Artificial Sequence
  CDR-H2 -- TM-37, TM-59
YVNPSSGYTKNNQKFKD

SEQ ID NO: 68 - protein, 17 aa, Artificial Sequence
  CDR-H2 -- TM-39
FINPSSGYTKYNQNFKD

SEQ ID NO: 69 - protein, 17 aa, Artificial Sequence
  CDR-H2 -- TM-41
RIDPEDGTTNFAPKFQD

SEQ ID NO: 70 - protein, 17 aa, Artificial Sequence
  CDR-H2 -- TM-42, TM-45
YINPSSGYTKYNQKFKD

SEQ ID NO: 71 - protein, 17 aa, Artificial Sequence
  CDR-H2 -- TM-44
YISSGSSTIYYADTVKG

SEQ ID NO: 72 - protein, 17 aa, Artificial Sequence
  CDR-H2 -- TM-46
QIYPGDGDTDYNGKFKD

SEQ ID NO: 73 - protein, 16 aa, Artificial Sequence
  CDR-H2 -- TM-47, TM-50
VIWAGGNTNYNSALMS

SEQ ID NO: 74 - protein, 17 aa, Artificial Sequence
  CDR-H2 -- TM-48, TM-60
WIYPRDGNTQYIEKLKG

SEQ ID NO: 75 - protein, 17 aa, Artificial Sequence
  CDR-H2 -- TM-49
NIDPSDSQTHYNQKFKD

SEQ ID NO: 76 - protein, 17 aa, Artificial Sequence
  CDR-H2 -- TM-51
NMFPGSSRTNYNEKFKS

SEQ ID NO: 77 - protein, 17 aa, Artificial Sequence
  CDR-H2 -- TM-52
NIDPSDSETHYNQKFKD

SEQ ID NO: 78 - protein, 13 aa, Artificial Sequence
  CDR-H3 -- TM-1
RGYTIYDFYAMDY

SEQ ID NO: 79 - protein, 3 aa, Artificial Sequence
  CDR-H3 -- TM-2
AGY

SEQ ID NO: 80 - protein, 9 aa, Artificial Sequence
  CDR-H3 -- TM-3
IAGGTGAAY

SEQ ID NO: 81 - protein, 10 aa, Artificial Sequence
  CDR-H3 -- TM-4
AATVVAGFDY

SEQ ID NO: 82 - protein, 9 aa, Artificial Sequence
  CDR-H3 -- TM-5
SKGSGTGDY

SEQ ID NO: 83 - protein, 11 aa, Artificial Sequence
  CDR-H3 -- TM-6
HEEDYSNWFPF

SEQ ID NO: 84 - protein, 9 aa, Artificial Sequence
  CDR-H3 -- TM-7
TYYANSPDY

SEQ ID NO: 85 - protein, 4 aa, Artificial Sequence
  CDR-H3 -- TM-8
RLAY

SEQ ID NO: 86 - protein, 10 aa, Artificial Sequence
  CDR-H3 -- TM-9
QNFYYGCEDY

SEQ ID NO: 87 - protein, 10 aa, Artificial Sequence
  CDR-H3 -- TM-10
WGHYDEAMDD

SEQ ID NO: 88 - protein, 9 aa, Artificial Sequence
  CDR-H3 -- TM-11, TM-24
SLRPLHFDY

SEQ ID NO: 89 - protein, 13 aa, Artificial Sequence
  CDR-H3 -- TM-12
RVIYDGYYRTMDC

SEQ ID NO: 90 - protein, 8 aa, Artificial Sequence
  CDR-H3 -- TM-13, TM-84
YFYGGMDY

SEQ ID NO: 91 - protein, 13 aa, Artificial Sequence
  CDR-H3 -- TM-14
SPYSSYVGYAVDY

SEQ ID NO: 92 - protein, 8 aa, Artificial Sequence
  CDR-H3 -- TM-15
KAYGGFPY

SEQ ID NO: 93 - protein, 6 aa, Artificial Sequence
  CDR-H3 -- TM-16, TM-20
SQPFTY

SEQ ID NO: 94 - protein, 13 aa, Artificial Sequence
  CDR-H3 -- TM-17
SSYGVYVAYPMDY

SEQ ID NO: 95 - protein, 14 aa, Artificial Sequence
  CDR-H3 -- TM-18
EGQLRLRRVYAMDY

SEQ ID NO: 96 - protein, 11 aa, Artificial Sequence
  CDR-H3 -- TM-19
RIGNLYHVMDY

SEQ ID NO: 97 - protein, 8 aa, Artificial Sequence
  CDR-H3 -- TM-21
DGGTGFTY

SEQ ID NO: 98 - protein, 12 aa, Artificial Sequence
  CDR-H3 -- TM-22
PYYGIRNCYFDV

SEQ ID NO: 99 - protein, 11 aa, Artificial Sequence
  CDR-H3 -- TM-23, TM-26
SDFITTVVADY

SEQ ID NO: 100 - protein, 10 aa, Artificial Sequence
  CDR-H3 -- TM-25
DNFYSYAMDY

SEQ ID NO: 101 - protein, 11 aa, Artificial Sequence
  CDR-H3 -- TM-27
IIYDHDWYFDV

SEQ ID NO: 102 - protein, 9 aa, Artificial Sequence
  CDR-H3 -- TM-28
SYFSNPIGY

SEQ ID NO: 103 - protein, 8 aa, Artificial Sequence
  CDR-H3 -- TM-29
HYTNPFAY

SEQ ID NO: 104 - protein, 8 aa, Artificial Sequence
  CDR-H3 -- TM-30
TLPQAMDY

SEQ ID NO: 105 - protein, 13 aa, Artificial Sequence
  CDR-H3 -- TM-31
SPPTVVLIGYFDY

SEQ ID NO: 106 - protein, 11 aa, Artificial Sequence
  CDR-H3 -- TM-32
RTWDLYYAVDN

SEQ ID NO: 107 - protein, 2 aa, Artificial Sequence
  CDR-H3 -- TM-33
SY

SEQ ID NO: 108 - protein, 10 aa, Artificial Sequence
  CDR-H3 -- TM-34
RDGNYWYFDV

SEQ ID NO: 109 - protein, 11 aa, Artificial Sequence
  CDR-H3 -- TM-35
SLYDYDGVFAY

SEQ ID NO: 110 - protein, 12 aa, Artificial Sequence
  CDR-H3 -- TM-37, TM-42, TM-45, TM-56, TM-59
EGGSISDWYFDV

SEQ ID NO: 111 - protein, 12 aa, Artificial Sequence
  CDR-H3 -- TM-39
EAGSISDWYFDV

SEQ ID NO: 112 - protein, 7 aa, Artificial Sequence
  CDR-H3 -- TM-41
EWDSGAY

SEQ ID NO: 113 - protein, 10 aa, Artificial Sequence
  CDR-H3 -- TM-44
NYGSPYAMDY

SEQ ID NO: 114 - protein, 9 aa, Artificial Sequence
  CDR-H3 -- TM-46
GDGFSYFDY

SEQ ID NO: 115 - protein, 12 aa, Artificial Sequence
  CDR-H3 -- TM-47, TM-50
EAKLLRSYAMDY

SEQ ID NO: 116 - protein, 8 aa, Artificial Sequence
  CDR-H3 -- TM-48, TM-60
WIFYAMDY

SEQ ID NO: 117 - protein, 10 aa, Artificial Sequence
  CDR-H3 -- TM-49
LITVDYAMDY

SEQ ID NO: 118 - protein, 17 aa, Artificial Sequence
  CDR-H3 -- TM-51
KEGLWTYGYDGGAWFAY

SEQ ID NO: 119 - protein, 11 aa, Artificial Sequence
  CDR-H3 -- TM-52
RGYYGRSPFAY

SEQ ID NO: 120 - protein, 16 aa, Artificial Sequence
  CDR-L1 -- TM-1
RSSQSIVYNNGNTYLE

SEQ ID NO: 121 - protein, 10 aa, Artificial Sequence
  CDR-L1 -- TM-2
RASSSVSYIH

SEQ ID NO: 122 - protein, 16 aa, Artificial Sequence
  CDR-L1 -- TM-3, TM-9
RSSKSLLHSNGITYLY

SEQ ID NO: 123 - protein, 14 aa, Artificial Sequence
  CDR-L1 -- TM-4
RSSTGAVTTSNYAN

SEQ ID NO: 124 - protein, 11 aa, Artificial Sequence
  CDR-L1 -- TM-5
RASENIYIYLA

SEQ ID NO: 125 - protein, 11 aa, Artificial Sequence
  CDR-L1 -- TM-6
KASDHINNWLA

SEQ ID NO: 126 - protein, 17 aa, Artificial Sequence
  CDR-L1 -- TM-7
KSSQSLLNSGNQRNYLA

SEQ ID NO: 127 - protein, 11 aa, Artificial Sequence
  CDR-L1 -- TM-8, TM-31
KASQDINSYLS

SEQ ID NO: 128 - protein, 16 aa, Artificial Sequence
  CDR-L1 -- TM-10
RSSQSLVHSNGKTYLH

SEQ ID NO: 129 - protein, 17 aa, Artificial Sequence
  CDR-L1 -- TM-11
KSSQSLLNSNNLQNYLA

SEQ ID NO: 130 - protein, 16 aa, Artificial Sequence
  CDR-L1 -- TM-12
RSSQTIVHSNGNTYLE

SEQ ID NO: 131 - protein, 11 aa, Artificial Sequence
  CDR-L1 -- TM-13, TM-84
RASENIYSSLG

SEQ ID NO: 132 - protein, 11 aa, Artificial Sequence
  CDR-L1 -- TM-14, TM-17
RASQDIGSNLN

SEQ ID NO: 133 - protein, 11 aa, Artificial Sequence
  CDR-L1 -- TM-15
RVSENIYNNLA

SEQ ID NO: 134 - protein, 10 aa, Artificial Sequence
  CDR-L1 -- TM-16, TM-20
SASSSLNYMY

SEQ ID NO: 135 - protein, 15 aa, Artificial Sequence
  CDR-L1 -- TM-18
RASKSVSISVYTYVH

SEQ ID NO: 136 - protein, 16 aa, Artificial Sequence
  CDR-L1 -- TM-19
RSSQSIVHSNGNTYLE

SEQ ID NO: 137 - protein, 16 aa, Artificial Sequence
  CDR-L1 -- TM-21, TM-25
RSSKSLLHSNGITYLF

SEQ ID NO: 138 - protein, 16 aa, Artificial Sequence
  CDR-L1 -- TM-22
RSGQSIVHSNGNTYLE

SEQ ID NO: 139 - protein, 16 aa, Artificial Sequence
  CDR-L1 -- TM-23, TM-26
RSSQSLTNYYGNTYLS

SEQ ID NO: 140 - protein, 17 aa, Artificial Sequence
  CDR-L1 -- TM-24
KSSQSLLNSNNQQNYLA

SEQ ID NO: 141 - protein, 10 aa, Artificial Sequence
  CDR-L1 -- TM-27
SASSSISYMY

SEQ ID NO: 142 - protein, 17 aa, Artificial Sequence
  CDR-L1 -- TM-28, TM-61, TM-86
KSSQSLLNSGNQKNYLA

SEQ ID NO: 143 - protein, 16 aa, Artificial Sequence
  CDR-L1 -- TM-29
RSSKSLLHYNGITYLY

SEQ ID NO: 144 - protein, 16 aa, Artificial Sequence
  CDR-L1 -- TM-30
RSSQTIVHRNGNTYLE

SEQ ID NO: 145 - protein, 16 aa, Artificial Sequence
  CDR-L1 -- TM-32
RSSQNIVHSNGNTYLE

SEQ ID NO: 146 - protein, 11 aa, Artificial Sequence
  CDR-L1 -- TM-33
KASQNVGTAVA

SEQ ID NO: 147 - protein, 10 aa, Artificial Sequence
  CDR-L1 -- TM-34
RATSSVTYMH

SEQ ID NO: 148 - protein, 16 aa, Artificial Sequence
  CDR-L1 -- TM-35
RSSQSIVHRNGNTYLE

SEQ ID NO: 149 - protein, 16 aa, Artificial Sequence
  CDR-L1 -- TM-37, TM-59
RSSKSLLHSNGNTYSY

SEQ ID NO: 150 - protein, 16 aa, Artificial Sequence
  CDR-L1 -- TM-39
RSSKTLLNSNGNTYLY

SEQ ID NO: 151 - protein, 17 aa, Artificial Sequence
  CDR-L1 -- TM-41, TM-44
KSSQSLLYSSNQKNYLA

SEQ ID NO: 152 - protein, 16 aa, Artificial Sequence
  CDR-L1 -- TM-42, TM-45
RSSKSLLHSNGNTYLY

SEQ ID NO: 153 - protein, 10 aa, Artificial Sequence
  CDR-L1 -- TM-46
CASSRVNYMH

SEQ ID NO: 154 - protein, 17 aa, Artificial Sequence
  CDR-L1 -- TM-47
KSSQSLLNSGNQKNYLT

SEQ ID NO: 155 - protein, 16 aa, Artificial Sequence
  CDR-L1 -- TM-48, TM-54, TM-60
RSSQSIVHGNGNTYLE

SEQ ID NO: 156 - protein, 10 aa, Artificial Sequence
  CDR-L1 -- TM-49, TM-62
SASSSVSYMY

SEQ ID NO: 157 - protein, 11 aa, Artificial Sequence
  CDR-L1 -- TM-50
RASQDINNYLY

SEQ ID NO: 158 - protein, 17 aa, Artificial Sequence
  CDR-L1 -- TM-51
KSSQSLRNSRTRKNYLA

SEQ ID NO: 159 - protein, 10 aa, Artificial Sequence
  CDR-L1 -- TM-52
SASSSVSYMH

SEQ ID NO: 160 - protein, 7 aa, Artificial Sequence
  CDR-L2 -- TM-1, TM-22, TM-30, TM-32, TM-35, TM-48, TM-54, TM-60, TM-69, TM-83, TM-94
KVSNRFS

SEQ ID NO: 161 - protein, 7 aa, Artificial Sequence
  CDR-L2 -- TM-2, TM-34
ATSNLAS

SEQ ID NO: 162 - protein, 7 aa, Artificial Sequence
  CDR-L2 -- TM-3
QMSSLAS

SEQ ID NO: 163 - protein, 7 aa, Artificial Sequence
  CDR-L2 -- TM-4
GTNNRAP

SEQ ID NO: 164 - protein, 7 aa, Artificial Sequence
  CDR-L2 -- TM-5
NGKMLAE

SEQ ID NO: 165 - protein, 7 aa, Artificial Sequence
  CDR-L2 -- TM-6
GATSLET

SEQ ID NO: 166 - protein, 7 aa, Artificial Sequence
  CDR-L2 -- TM-7, TM-28, TM-61, TM-86
GASTRES

SEQ ID NO: 167 - protein, 7 aa, Artificial Sequence
  CDR-L2 -- TM-8
RANRLVD

SEQ ID NO: 168 - protein, 7 aa, Artificial Sequence
  CDR-L2 -- TM-9, TM-21, TM-25, TM-29
QMSNLAS

SEQ ID NO: 169 - protein, 7 aa, Artificial Sequence
  CDR-L2 -- TM-10, TM-12
KISNRFS

SEQ ID NO: 170 - protein, 7 aa, Artificial Sequence
  CDR-L2 -- TM-11, TM-24
FASIRES

SEQ ID NO: 171 - protein, 7 aa, Artificial Sequence
  CDR-L2 -- TM-13, TM-15, TM-84
AATNLAD

SEQ ID NO: 172 - protein, 7 aa, Artificial Sequence
  CDR-L2 -- TM-14, TM-17, TM-89
ATSSLDS

SEQ ID NO: 173 - protein, 7 aa, Artificial Sequence
  CDR-L2 -- TM-16, TM-20, TM-73
DTSNLAS

SEQ ID NO: 174 - protein, 7 aa, Artificial Sequence
  CDR-L2 -- TM-18
LASNLES

SEQ ID NO: 175 - protein, 7 aa, Artificial Sequence
  CDR-L2 -- TM-19
KVFNRFS

SEQ ID NO: 176 - protein, 7 aa, Artificial Sequence
  CDR-L2 -- TM-23, TM-26, TM-66, TM-82
GISNRFS

SEQ ID NO: 177 - protein, 7 aa, Artificial Sequence
  CDR-L2 -- TM-27
RTSTLAS

SEQ ID NO: 178 - protein, 7 aa, Artificial Sequence
  CDR-L2 -- TM-31
RGNGLVD

SEQ ID NO: 179 - protein, 7 aa, Artificial Sequence
  CDR-L2 -- TM-33
SASNRYT

SEQ ID NO: 180 - protein, 7 aa, Artificial Sequence
  CDR-L2 -- TM-37, TM-39, TM-42, TM-45, TM-56, TM-59, TM-63, TM-64, TM-68, TM-78, TM-80
RMSNLAS

SEQ ID NO: 181 - protein, 7 aa, Artificial Sequence
  CDR-L2 -- TM-41, TM-44, TM-47, TM-51
WASTRES

SEQ ID NO: 182 - protein, 7 aa, Artificial Sequence
  CDR-L2 -- TM-46, TM-49, TM-52, TM-62
DTSKLAS

SEQ ID NO: 183 - protein, 7 aa, Artificial Sequence
  CDR-L2 -- TM-50
YTSMLHS

SEQ ID NO: 184 - protein, 9 aa, Artificial Sequence
  CDR-L3 -- TM-1
FQVSHVPFT

SEQ ID NO: 185 - protein, 9 aa, Artificial Sequence
  CDR-L3 -- TM-2
QQWSSNPST

SEQ ID NO: 186 - protein, 9 aa, Artificial Sequence
  CDR-L3 -- TM-3, TM-9
AQNLELPWT

SEQ ID NO: 187 - protein, 9 aa, Artificial Sequence
  CDR-L3 -- TM-4
VLWYSNHLV

SEQ ID NO: 188 - protein, 9 aa, Artificial Sequence
  CDR-L3 -- TM-5
QHHYGSPPA

SEQ ID NO: 189 - protein, 9 aa, Artificial Sequence
  CDR-L3 -- TM-6
QQYWSSPYT

SEQ ID NO: 190 - protein, 9 aa, Artificial Sequence
  CDR-L3 -- TM-7, TM-28, TM-86
QNDHSYPLT

SEQ ID NO: 191 - protein, 9 aa, Artificial Sequence
  CDR-L3 -- TM-8
LQYDEFPLT

SEQ ID NO: 192 - protein, 9 aa, Artificial Sequence
  CDR-L3 -- TM-10
SQITHVPWT

SEQ ID NO: 193 - protein, 9 aa, Artificial Sequence
  CDR-L3 -- TM-11
QQHYNTPFT

SEQ ID NO: 194 - protein, 9 aa, Artificial Sequence
  CDR-L3 -- TM-12, TM-35
FQGSHVPYT

SEQ ID NO: 195 - protein, 9 aa, Artificial Sequence
  CDR-L3 -- TM-13
QHLWSIPWT

SEQ ID NO: 196 - protein, 9 aa, Artificial Sequence
  CDR-L3 -- TM-14, TM-17, TM-89
LQYASSPRT

SEQ ID NO: 197 - protein, 9 aa, Artificial Sequence
  CDR-L3 -- TM-15
QHFWDTPFT

SEQ ID NO: 198 - protein, 9 aa, Artificial Sequence
  CDR-L3 -- TM-16, TM-20
QQWTSFPPT

SEQ ID NO: 199 - protein, 9 aa, Artificial Sequence
  CDR-L3 -- TM-18
QHSRELPYT

SEQ ID NO: 200 - protein, 9 aa, Artificial Sequence
  CDR-L3 -- TM-19, TM-32
FQGSHVPFT

SEQ ID NO: 201 - protein, 9 aa, Artificial Sequence
  CDR-L3 -- TM-21
VQNLELPYT

SEQ ID NO: 202 - protein, 9 aa, Artificial Sequence
  CDR-L3 -- TM-22, TM-54
FQGSHVPWT

SEQ ID NO: 203 - protein, 9 aa, Artificial Sequence
  CDR-L3 -- TM-23, TM-26
LQGTHQPRT

SEQ ID NO: 204 - protein, 9 aa, Artificial Sequence
  CDR-L3 -- TM-24, TM-81
QQHYSTPFT

SEQ ID NO: 205 - protein, 8 aa, Artificial Sequence
  CDR-L3 -- TM-25
AQNLELWT

SEQ ID NO: 206 - protein, 9 aa, Artificial Sequence
  CDR-L3 -- TM-27
QQYHSYPRT

SEQ ID NO: 207 - protein, 9 aa, Artificial Sequence
  CDR-L3 -- TM-29
AQNLELPYT

SEQ ID NO: 208 - protein, 9 aa, Artificial Sequence
  CDR-L3 -- TM-30
FQGSHLPWT

SEQ ID NO: 209 - protein, 9 aa, Artificial Sequence
  CDR-L3 -- TM-31
LQYDEFPFT

SEQ ID NO: 210 - protein, 9 aa, Artificial Sequence
  CDR-L3 -- TM-33
QQYSSYPYT

SEQ ID NO: 211 - protein, 9 aa, Artificial Sequence
  CDR-L3 -- TM-34
QQWSSNPYT

SEQ ID NO: 212 - protein, 9 aa, Artificial Sequence
  CDR-L3 -- TM-37, TM-42, TM-45, TM-56, TM-59
MQHLEYPYT

SEQ ID NO: 213 - protein, 9 aa, Artificial Sequence
  CDR-L3 -- TM-39
MQHLDYPYT

SEQ ID NO: 214 - protein, 9 aa, Artificial Sequence
  CDR-L3 -- TM-41
QQYYSYPYT

SEQ ID NO: 215 - protein, 8 aa, Artificial Sequence
  CDR-L3 -- TM-44
QQYYSYPT

SEQ ID NO: 216 - protein, 9 aa, Artificial Sequence
  CDR-L3 -- TM-46
QQWSSNPPT

SEQ ID NO: 217 - protein, 9 aa, Artificial Sequence
  CDR-L3 -- TM-47
QNDYSYPLT

SEQ ID NO: 218 - protein, 9 aa, Artificial Sequence
  CDR-L3 -- TM-48, TM-60
FQGSHLPYT

SEQ ID NO: 219 - protein, 9 aa, Artificial Sequence
  CDR-L3 -- TM-49, TM-62
QQYHSYPPT

SEQ ID NO: 220 - protein, 9 aa, Artificial Sequence
  CDR-L3 -- TM-50
QQGSTLMYT

SEQ ID NO: 221 - protein, 8 aa, Artificial Sequence
  CDR-L3 -- TM-51
KQSYNLLT

SEQ ID NO: 222 - protein, 9 aa, Artificial Sequence
  CDR-L3 -- TM-52
FQGSGYPLT

SEQ ID NO: 223 - protein, 5 aa, Artificial Sequence
  CDR-H1 -- TM-54
TYDLN

SEQ ID NO: 224 - protein, 5 aa, Artificial Sequence
  CDR-H1 -- TM-61
NYWMN

SEQ ID NO: 225 - protein, 5 aa, Artificial Sequence
  CDR-H1 -- TM-62
RYWMH

SEQ ID NO: 226 - protein, 5 aa, Artificial Sequence
  CDR-H1 -- TM-63
SFWMH

SEQ ID NO: 227 - protein, 5 aa, Artificial Sequence
  CDR-H1 -- TM-65
SSWMH

SEQ ID NO: 228 - protein, 5 aa, Artificial Sequence
  CDR-H1 -- TM-68
DDYMH

SEQ ID NO: 229 - protein, 5 aa, Artificial Sequence
  CDR-H1 -- TM-69
SYGVH

SEQ ID NO: 230 - protein, 5 aa, Artificial Sequence
  CDR-H1 -- TM-73
SHWMH

SEQ ID NO: 231 - protein, 5 aa, Artificial Sequence
  CDR-H1 -- TM-74
SYVIH

SEQ ID NO: 232 - protein, 5 aa, Artificial Sequence
  CDR-H1 -- TM-75
SYVMH

SEQ ID NO: 233 - protein, 7 aa, Artificial Sequence
  CDR-H1 -- TM-76
TSGMGVS

SEQ ID NO: 234 - protein, 5 aa, Artificial Sequence
  CDR-H1 -- TM-77
DYEMH

SEQ ID NO: 235 - protein, 5 aa, Artificial Sequence
  CDR-H1 -- TM-78
NHWMH

SEQ ID NO: 236 - protein, 5 aa, Artificial Sequence
  CDR-H1 -- TM-79
NYVMH

SEQ ID NO: 237 - protein, 5 aa, Artificial Sequence
  CDR-H1 -- TM-80
TFWMH

SEQ ID NO: 238 - protein, 5 aa, Artificial Sequence
  CDR-H1 -- TM-83
TYWIH

SEQ ID NO: 239 - protein, 5 aa, Artificial Sequence
  CDR-H1 -- TM-85
GYWMH

SEQ ID NO: 240 - protein, 6 aa, Artificial Sequence
  CDR-H1 -- TM-91
SGYYWD

SEQ ID NO: 241 - protein, 5 aa, Artificial Sequence
  CDR-H1 -- TM-92
DYTMD

SEQ ID NO: 242 - protein, 5 aa, Artificial Sequence
  CDR-H1 -- TM-93
SYGIS

SEQ ID NO: 243 - protein, 5 aa, Artificial Sequence
  CDR-H1 -- TM-94
DYNMD

SEQ ID NO: 244 - protein, 17 aa, Artificial Sequence
  CDR-H2 -- TM-54
WIYPRGGSTEYNEKFKG

SEQ ID NO: 245 - protein, 17 aa, Artificial Sequence
  CDR-H2 -- TM-56
YINPSSGYSKYNQRFKD

SEQ ID NO: 246 - protein, 17 aa, Artificial Sequence
  CDR-H2 -- TM-61
YINPSSDYTKYNQNFKD

SEQ ID NO: 247 - protein, 17 aa, Artificial Sequence
  CDR-H2 -- TM-62
NIDPSDSETQYNPKFKD

SEQ ID NO: 248 - protein, 17 aa, Artificial Sequence
  CDR-H2 -- TM-63
NIDPSDSETHYSQKFKD

SEQ ID NO: 249 - protein, 17 aa, Artificial Sequence
  CDR-H2 -- TM-64
NIDPSDSDTHYNQNFRG

SEQ ID NO: 250 - protein, 17 aa, Artificial Sequence
  CDR-H2 -- TM-65
EINPKNGGTNYNEKFKT

SEQ ID NO: 251 - protein, 17 aa, Artificial Sequence
  CDR-H2 -- TM-66
TIGSVGSYTYYPDSVKG

SEQ ID NO: 252 - protein, 17 aa, Artificial Sequence
  CDR-H2 -- TM-68
WIDPENGDTEYASKFQG

SEQ ID NO: 253 - protein, 16 aa, Artificial Sequence
  CDR-H2 -- TM-69
IMGWDDKKYYNSALKS

SEQ ID NO: 254 - protein, 17 aa, Artificial Sequence
  CDR-H2 -- TM-70, TM-71
TIGYGGSYSYYPDSVKG

SEQ ID NO: 255 - protein, 17 aa, Artificial Sequence
  CDR-H2 -- TM-72
NIDPSDSDTHYNQKFKD

SEQ ID NO: 256 - protein, 17 aa, Artificial Sequence
  CDR-H2 -- TM-73
EIDPSDSYTYYNQKFKG

SEQ ID NO: 257 - protein, 17 aa, Artificial Sequence
  CDR-H2 -- TM-74
FINPNNDGTKYNEKFRG

SEQ ID NO: 258 - protein, 17 aa, Artificial Sequence
  CDR-H2 -- TM-75
YINPYNDGTKYNEKFKG

SEQ ID NO: 259 - protein, 16 aa, Artificial Sequence
  CDR-H2 -- TM-76
HIFWDDDKRYNLFLKS

SEQ ID NO: 260 - protein, 17 aa, Artificial Sequence
  CDR-H2 -- TM-77
VIDPETGGTAYNQMFKG

SEQ ID NO: 261 - protein, 17 aa, Artificial Sequence
  CDR-H2 -- TM-78
NIDPSDSDTHYNHKFKD

SEQ ID NO: 262 - protein, 17 aa, Artificial Sequence
  CDR-H2 -- TM-79
HITPHNDGTKYNEKFKG

SEQ ID NO: 263 - protein, 17 aa, Artificial Sequence
  CDR-H2 -- TM-80
NIDPSDSETHYNQEFKD

SEQ ID NO: 264 - protein, 17 aa, Artificial Sequence
  CDR-H2 -- TM-81
NIDPSDSEIHYNQKFKD

SEQ ID NO: 265 - protein, 17 aa, Artificial Sequence
  CDR-H2 -- TM-82
VINPGSGITDYSEKFKG

SEQ ID NO: 266 - protein, 17 aa, Artificial Sequence
  CDR-H2 -- TM-83
NINPSNGNTKNNERFKT

SEQ ID NO: 267 - protein, 17 aa, Artificial Sequence
  CDR-H2 -- TM-85
RIDPYSGDTRYNEKFKN

SEQ ID NO: 268 - protein, 17 aa, Artificial Sequence
  CDR-H2 -- TM-86
VISPYNDNTNYNQKFKG

SEQ ID NO: 269 - protein, 17 aa, Artificial Sequence
  CDR-H2 -- TM-87
VINPYNGATSYNQKFQD

SEQ ID NO: 270 - protein, 17 aa, Artificial Sequence
  CDR-H2 -- TM-88
VINPYSGATYYTQKFKG

SEQ ID NO: 271 - protein, 17 aa, Artificial Sequence
  CDR-H2 -- TM-89
VINPGSGGTDYNEKFKG

SEQ ID NO: 272 - protein, 17 aa, Artificial Sequence
  CDR-H2 -- TM-90
WIYPRDGNAKYNEKFKG

SEQ ID NO: 273 - protein, 16 aa, Artificial Sequence
  CDR-H2 -- TM-91
YISYDGNNNYNPSLKN

SEQ ID NO: 274 - protein, 17 aa, Artificial Sequence
  CDR-H2 -- TM-92
HINPNDGGTFYNQKFKG

SEQ ID NO: 275 - protein, 16 aa, Artificial Sequence
  CDR-H2 -- TM-93
VIWTGGGTNYNSALES

SEQ ID NO: 276 - protein, 17 aa, Artificial Sequence
  CDR-H2 -- TM-94
DINPYNGGTLYNQKFKG

SEQ ID NO: 277 - protein, 8 aa, Artificial Sequence
  CDR-H3 -- TM-54
YSIYAMDY

SEQ ID NO: 278 - protein, 12 aa, Artificial Sequence
  CDR-H3 -- TM-61
WGGSRSYWYFDV

SEQ ID NO: 279 - protein, 10 aa, Artificial Sequence
  CDR-H3 -- TM-62
LTTLDYAMDY

SEQ ID NO: 280 - protein, 9 aa, Artificial Sequence
  CDR-H3 -- TM-63, TM-80
SSNWDNFDY

SEQ ID NO: 281 - protein, 14 aa, Artificial Sequence
  CDR-H3 -- TM-64
SRGGTYDYGEAMDY

SEQ ID NO: 282 - protein, 6 aa, Artificial Sequence
  CDR-H3 -- TM-65
PTWFTY

SEQ ID NO: 283 - protein, 9 aa, Artificial Sequence
  CDR-H3 -- TM-66
QEGSSHFDY

SEQ ID NO: 284 - protein, 7 aa, Artificial Sequence
  CDR-H3 -- TM-68
PQTATTC

SEQ ID NO: 285 - protein, 9 aa, Artificial Sequence
  CDR-H3 -- TM-69
EAGPYGMDY

SEQ ID NO: 286 - protein, 12 aa, Artificial Sequence
  CDR-H3 -- TM-70, TM-71
QDSNYEGVWFAY

SEQ ID NO: 287 - protein, 14 aa, Artificial Sequence
  CDR-H3 -- TM-72, TM-78
SRGGYYDYGEAMDY

SEQ ID NO: 288 - protein, 11 aa, Artificial Sequence
  CDR-H3 -- TM-73
NYHGSGPPFAH

SEQ ID NO: 289 - protein, 12 aa, Artificial Sequence
  CDR-H3 -- TM-74
PYYDYEGYTMDY

SEQ ID NO: 290 - protein, 10 aa, Artificial Sequence
  CDR-H3 -- TM-75
GGWFPYAMDY

SEQ ID NO: 291 - protein, 10 aa, Artificial Sequence
  CDR-H3 -- TM-76
MGRQRDYFDY

SEQ ID NO: 292 - protein, 6 aa, Artificial Sequence
  CDR-H3 -- TM-77
YGIEGY

SEQ ID NO: 293 - protein, 12 aa, Artificial Sequence
  CDR-H3 -- TM-79
SGQFGRGDYFHY

SEQ ID NO: 294 - protein, 9 aa, Artificial Sequence
  CDR-H3 -- TM-81
SPAGQILDY

SEQ ID NO: 295 - protein, 11 aa, Artificial Sequence
  CDR-H3 -- TM-82
SPFITTVVADY

SEQ ID NO: 296 - protein, 10 aa, Artificial Sequence
  CDR-H3 -- TM-83
GDYYGTSYPF

SEQ ID NO: 297 - protein, 9 aa, Artificial Sequence
  CDR-H3 -- TM-85
YGLGHAMDY

SEQ ID NO: 298 - protein, 9 aa, Artificial Sequence
  CDR-H3 -- TM-86
TYYSNYFDY

SEQ ID NO: 299 - protein, 9 aa, Artificial Sequence
  CDR-H3 -- TM-87
GNYDWYFDV

SEQ ID NO: 300 - protein, 12 aa, Artificial Sequence
  CDR-H3 -- TM-88
GGYDYGHWYFDV

SEQ ID NO: 301 - protein, 13 aa, Artificial Sequence
  CDR-H3 -- TM-89
DPYSKYVHYPMDY

SEQ ID NO: 302 - protein, 12 aa, Artificial Sequence
  CDR-H3 -- TM-90
VSDYHGGYGMDY

SEQ ID NO: 303 - protein, 9 aa, Artificial Sequence
  CDR-H3 -- TM-91
AVYSVGFAY

SEQ ID NO: 304 - protein, 16 aa, Artificial Sequence
  CDR-H3 -- TM-92
WVYYYGISYEGGAMDY

SEQ ID NO: 305 - protein, 15 aa, Artificial Sequence
  CDR-H3 -- TM-93
NPPKIFYDYLMYFDY

SEQ ID NO: 306 - protein, 9 aa, Artificial Sequence
  CDR-H3 -- TM-94
VGYHGTPDY

SEQ ID NO: 307 - protein, 16 aa, Artificial Sequence
  CDR-L1 -- TM-56
RSSKSLLQNNGNTYLY

SEQ ID NO: 308 - protein, 16 aa, Artificial Sequence
  CDR-L1 -- TM-63
RSSQSLLHVNGNTYLY

SEQ ID NO: 309 - protein, 16 aa, Artificial Sequence
  CDR-L1 -- TM-64, TM-71, TM-72
RSSKSLLHTNGNTYLF

SEQ ID NO: 310 - protein, 11 aa, Artificial Sequence
  CDR-L1 -- TM-65
RASQNINIWLS

SEQ ID NO: 311 - protein, 16 aa, Artificial Sequence
  CDR-L1 -- TM-66
RSSQSLVNSYGKTFLS

SEQ ID NO: 312 - protein, 16 aa, Artificial Sequence
  CDR-L1 -- TM-68
SSSKSLLHSSGITYLY

SEQ ID NO: 313 - protein, 16 aa, Artificial Sequence
  CDR-L1 -- TM-69
RSSQSLVHSNGNTYLH

SEQ ID NO: 314 - protein, 15 aa, Artificial Sequence
  CDR-L1 -- TM-70
RASESVDNYGISFMN

SEQ ID NO: 315 - protein, 10 aa, Artificial Sequence
  CDR-L1 -- TM-73
SASSGISYIY

SEQ ID NO: 316 - protein, 11 aa, Artificial Sequence
  CDR-L1 -- TM-74
KASQNVRSAVA

SEQ ID NO: 317 - protein, 15 aa, Artificial Sequence
  CDR-L1 -- TM-75
RASQSVDYNGISYMH

SEQ ID NO: 318 - protein, 10 aa, Artificial Sequence
  CDR-L1 -- TM-76
SASLSISYVH

SEQ ID NO: 319 - protein, 16 aa, Artificial Sequence
  CDR-L1 -- TM-77
KSSQSLFHSNGKTYLN

SEQ ID NO: 320 - protein, 16 aa, Artificial Sequence
  CDR-L1 -- TM-78
RSSKSLLHVNGNTYLF

SEQ ID NO: 321 - protein, 15 aa, Artificial Sequence
  CDR-L1 -- TM-79
RASKSVSTSGSIYIH

SEQ ID NO: 322 - protein, 16 aa, Artificial Sequence
  CDR-L1 -- TM-80
RSSQSLLHVNGHTYLY

SEQ ID NO: 323 - protein, 17 aa, Artificial Sequence
  CDR-L1 -- TM-81
KSSQSLLNSKNQKNYLA

SEQ ID NO: 324 - protein, 16 aa, Artificial Sequence
  CDR-L1 -- TM-82
RSSQSLANTYGNTYLS

SEQ ID NO: 325 - protein, 16 aa, Artificial Sequence
  CDR-L1 -- TM-83
RSSQSLVHSNGITYLH

SEQ ID NO: 326 - protein, 11 aa, Artificial Sequence
  CDR-L1 -- TM-85
KASQNVGTNVA

SEQ ID NO: 327 - protein, 11 aa, Artificial Sequence
  CDR-L1 -- TM-87
RASRSISKYLA

SEQ ID NO: 328 - protein, 11 aa, Artificial Sequence
  CDR-L1 -- TM-88
ITSSDIDDHMN

SEQ ID NO: 329 - protein, 11 aa, Artificial Sequence
  CDR-L1 -- TM-89
RASQDIGSSLN

SEQ ID NO: 330 - protein, 16 aa, Artificial Sequence
  CDR-L1 -- TM-90
RSSQSFVHGNGNTYLE

SEQ ID NO: 331 - protein, 11 aa, Artificial Sequence
  CDR-L1 -- TM-91
KASQNVYTNVA

SEQ ID NO: 332 - protein, 11 aa, Artificial Sequence
  CDR-L1 -- TM-92
RASKSISKYLA

SEQ ID NO: 333 - protein, 11 aa, Artificial Sequence
  CDR-L1 -- TM-93
KASQDVGSAVA

SEQ ID NO: 334 - protein, 16 aa, Artificial Sequence
  CDR-L1 -- TM-94
RSSQSLVYSNGNTYLH

SEQ ID NO: 335 - protein, 7 aa, Artificial Sequence
  CDR-L2 -- TM-65
KASNLHT

SEQ ID NO: 336 - protein, 7 aa, Artificial Sequence
  CDR-L2 -- TM-70
AASNQGS

SEQ ID NO: 337 - protein, 7 aa, Artificial Sequence
  CDR-L2 -- TM-71, TM-72
RKSNLAS

SEQ ID NO: 338 - protein, 7 aa, Artificial Sequence
  CDR-L2 -- TM-74
LASNRHT

SEQ ID NO: 339 - protein, 7 aa, Artificial Sequence
  CDR-L2 -- TM-75
AASNLES

SEQ ID NO: 340 - protein, 7 aa, Artificial Sequence
  CDR-L2 -- TM-76
GTSNLAS

SEQ ID NO: 341 - protein, 7 aa, Artificial Sequence
  CDR-L2 -- TM-77
MVSKLES

SEQ ID NO: 342 - protein, 7 aa, Artificial Sequence
  CDR-L2 -- TM-79
LTSNLES

SEQ ID NO: 343 - protein, 7 aa, Artificial Sequence
  CDR-L2 -- TM-81
FAFFRES

SEQ ID NO: 344 - protein, 7 aa, Artificial Sequence
  CDR-L2 -- TM-85, TM-91
SASYRYS

SEQ ID NO: 345 - protein, 7 aa, Artificial Sequence
  CDR-L2 -- TM-87, TM-92
SGSTLQS

SEQ ID NO: 346 - protein, 7 aa, Artificial Sequence
  CDR-L2 -- TM-88
EGNALRP

SEQ ID NO: 347 - protein, 7 aa, Artificial Sequence
  CDR-L2 -- TM-90
KVSHRFS

SEQ ID NO: 348 - protein, 7 aa, Artificial Sequence
  CDR-L2 -- TM-93
WSSTRLP

SEQ ID NO: 349 - protein, 9 aa, Artificial Sequence
  CDR-L3 -- TM-61
QNDSNYPFT

SEQ ID NO: 350 - protein, 9 aa, Artificial Sequence
  CDR-L3 -- TM-63
MQHLQYPFS

SEQ ID NO: 351 - protein, 9 aa, Artificial Sequence
  CDR-L3 -- TM-64, TM-71, TM-72, TM-78
MQHLEYPFT

SEQ ID NO: 352 - protein, 9 aa, Artificial Sequence
  CDR-L3 -- TM-65
LQGQSYPYT

SEQ ID NO: 353 - protein, 9 aa, Artificial Sequence
  CDR-L3 -- TM-66
LQGTHPPLT

SEQ ID NO: 354 - protein, 9 aa, Artificial Sequence
  CDR-L3 -- TM-68
AQMLERPWT

SEQ ID NO: 355 - protein, 9 aa, Artificial Sequence
  CDR-L3 -- TM-69
SQNTHVPLT

SEQ ID NO: 356 - protein, 9 aa, Artificial Sequence
  CDR-L3 -- TM-70
QQSKDVPWT

SEQ ID NO: 357 - protein, 9 aa, Artificial Sequence
  CDR-L3 -- TM-73
HQWRSYPPT

SEQ ID NO: 358 - protein, 9 aa, Artificial Sequence
  CDR-L3 -- TM-74
LQHWKYPFT

SEQ ID NO: 359 - protein, 9 aa, Artificial Sequence
  CDR-L3 -- TM-75
QQSIEDPWT

SEQ ID NO: 360 - protein, 8 aa, Artificial Sequence
  CDR-L3 -- TM-76
HQWSIYRT

SEQ ID NO: 361 - protein, 9 aa, Artificial Sequence
  CDR-L3 -- TM-77
LQVTHFPLT

SEQ ID NO: 362 - protein, 9 aa, Artificial Sequence
  CDR-L3 -- TM-79
QHSGELPWT

SEQ ID NO: 363 - protein, 9 aa, Artificial Sequence
  CDR-L3 -- TM-80
MQHLQYPFT

SEQ ID NO: 364 - protein, 9 aa, Artificial Sequence
  CDR-L3 -- TM-82
LQGTHPPRT

SEQ ID NO: 365 - protein, 9 aa, Artificial Sequence
  CDR-L3 -- TM-83
SQSTHVPYT

SEQ ID NO: 366 - protein, 9 aa, Artificial Sequence
  CDR-L3 -- TM-84
QHLWSVPWT

SEQ ID NO: 367 - protein, 9 aa, Artificial Sequence
  CDR-L3 -- TM-85, TM-91
QQYNSYPLT

SEQ ID NO: 368 - protein, 9 aa, Artificial Sequence
  CDR-L3 -- TM-87
QQHNEYPLT

SEQ ID NO: 369 - protein, 9 aa, Artificial Sequence
  CDR-L3 -- TM-88
LQSDNLPLT

SEQ ID NO: 370 - protein, 9 aa, Artificial Sequence
  CDR-L3 -- TM-90
FQNSHVPHT

SEQ ID NO: 371 - protein, 9 aa, Artificial Sequence
  CDR-L3 -- TM-92
QQHNEYPWT

SEQ ID NO: 372 - protein, 9 aa, Artificial Sequence
  CDR-L3 -- TM-93
QQYTSYPLT

SEQ ID NO: 373 - protein, 8 aa, Artificial Sequence
  CDR-L3 -- TM-94
SQSTHVYT

SEQ ID NO: 374 - protein, 122 aa, Artificial Sequence
  Variable Heavy Chain -- TM-1
QVQLQQSGAELVRPGTSVKVSCKASGYAFTNYLIEWVKQRPGQGLEWIGVINPGSGGTKYNEKLKGKATLTADKSSSTAYMQLSSLTSVDSAVYFCARRGYTIYDFYAMDYWGQGTSVTVSS

SEQ ID NO: 375 - protein, 112 aa, Artificial Sequence
  Variable Heavy Chain -- TM-2
HIQLVQSGPELKKPGETVKISCKASGYTFTEYPMHWVKQAPGKGFRWMGMIYTNTGEPTYAAEFKGRFAFSLETSASTGYLQINNLKNEDSATYFCVTAGYWGQGTLVTVSA

SEQ ID NO: 376 - protein, 119 aa, Artificial Sequence
  Variable Heavy Chain -- TM-3
QVTLKESGPGIVQPSQTLSLTCSFSGFSLNTLGRGVGWIRQPSGKGLEWLAKIWWNDDKFYYPALKSRLTISKDTSKNQIFLKIANVDTADSATYYCARIAGGTGAAYWGQGTTLTVSS

SEQ ID NO: 377 - protein, 119 aa, Artificial Sequence
  Variable Heavy Chain -- TM-4
EVQVQQSGPVLVKPGASVKMSCKASGYTFTDYYMNWVKQSHGKNLEWIGVINPYNGGTSYHQKFKGKATLTVDKSSSTAYMELNSLTSEDSAVYYCARAATVVAGFDYWGQGTTLTVSS

SEQ ID NO: 378 - protein, 118 aa, Artificial Sequence
  Variable Heavy Chain -- TM-5
QVQLQQSGTELARPGASVKVSCKASGYIFTTYGITWVKQRGGQGLEWIGEIYPRSDNTYYNEKFKDKATLTADKSSSTAYMELRSLTSEDSAVYFCARSKGSGTGDYWGQGTTVTVSS

SEQ ID NO: 379 - protein, 120 aa, Artificial Sequence
  Variable Heavy Chain -- TM-6
QVQLQQSGAELVKPGASVKLSCKASGYTFTEYTIHWVKQRSGQGLEWIGWFYPGSTYIDYNEKFKDKATLTADKSSSTVYLELSRLTSEDSAVYFCARHEEDYSNWFPFWGQGTLVTVSA

SEQ ID NO: 380 - protein, 118 aa, Artificial Sequence
  Variable Heavy Chain -- TM-7
EVQLQQSGPVLVKPGPPVKISCKASGFTFTDYYIHWVKLSHGKSLEWIGLVYPYNGDTDYNQKFKGKATLTVDTSSSTAYMELNSLTSEDSAVYYCARTYYANSPDYWGQGTTVTVSS

SEQ ID NO: 381 - protein, 113 aa, Artificial Sequence
  Variable Heavy Chain -- TM-8
QIQLVQSGPELKKPGETVKISCKASGYTFTDYPMHWVKQAPGKGFKWMGVIYTDTGEPKYAEVFKGRFAFSLETSASTAYLQINNLKNEDTATYFCVRRLAYWGQGTLVTVSA

SEQ ID NO: 382 - protein, 119 aa, Artificial Sequence
  Variable Heavy Chain -- TM-9
EVQLVESGGDLVKPGGSLKLSCVASGFTFSGYGMSWVRQTPDKRLEWVATISSGSFYIYYPDSVKGRLTVSRDNAKNTLYLQMSSLKSEDTAIYYCARQNFYYGCEDYWGQGTTLTVSS

SEQ ID NO: 383 - protein, 119 aa, Artificial Sequence
  Variable Heavy Chain -- TM-10
QVQLQQSGAELVKPGASVKISCKGSGYAFSSYWMNWVKQRPGKGLEWIGQIYPGDGDTNYNGKFKGKATLTADKSSTTAYIHLSSLTSEDSAVYFCARWGHYDEAMDDWGQGTSVTVSS

SEQ ID NO: 384 - protein, 117 aa, Artificial Sequence
  Variable Heavy Chain -- TM-11, TM-24
QVQLKQSGPGQVQPSQSLSITCTVSGFSLSDYGVHWVRQSPGKGLEWLGVIWNNGNTDYNAAFISRLSINKDNSKSQVFFKMTSLQADDTAIYYCVRSLRPLHFDYWGQGTTVTVSS

SEQ ID NO: 385 - protein, 122 aa, Artificial Sequence
  Variable Heavy Chain -- TM-12
EVQLQQSGAELMRPGASVKLSCTASGFNIQDYYMYWVKQRPEQGLEWIGRIDPEDGDAEYAPKFQGKATMTADTSSNTAYLQLSSLTSEDTAVYYCSTRVIYDGYYRTMDCWGQGTSVTVSS

SEQ ID NO: 386 - protein, 117 aa, Artificial Sequence
  Variable Heavy Chain -- TM-13
QVQLQQSGAELVKPGASVKMSCKASGYTFTTFPIEWMKQSHGKGLEWIGNFHPYNDDTKYNEKFKGKATLTVDKSSSTVYLDLSRLTSDDSAVYYCARYFYGGMDYWGQGTSVTVSS

SEQ ID NO: 387 - protein, 122 aa, Artificial Sequence
  Variable Heavy Chain -- TM-14
QVQLQQSGPELVRPGTSVKVSCKASGYAFTNYLIEWVKQRPGQGLEWIGVINPGGGNTDYSEKFKDKATLTADKSSNTAYIQLSSLTSEDSAVYFCARSPYSSYVGYAVDYWGQGTSVTVSS

SEQ ID NO: 388 - protein, 117 aa, Artificial Sequence
  Variable Heavy Chain -- TM-15
QVQLQQPGAEFVRPGASVKLSCKASGYTFTNYWITWVKQRPGHGLEWIGDIYPGSGNSNYNESFKRKATLTVDTSSSTAYMHLSSQTSEDSAVYFCARKAYGGFPYWGQGTLVTVSA

SEQ ID NO: 389 - protein, 115 aa, Artificial Sequence
  Variable Heavy Chain -- TM-16, TM-20
EVQLQQSGAELVRPGASVKLSCTTSGFNIKDYYMHWVKQRTEQGLEWIGRIDPEDGETKYAPEFQGKATITSDTSSNTAFLQLSSLTSEDTAVYYCASSQPFTYWGQGTLVTVSA

SEQ ID NO: 390 - protein, 122 aa, Artificial Sequence
  Variable Heavy Chain -- TM-17
QVQLQQSGAELIRPGTSVKVSCKASGYAFTDYLIEWVKQRPGQGLEWIGVINPGSGGTNYNEKFKGKAKLTADKSSSTAYMQLSSLTSEDSAVYFCVRSSYGVYVAYPMDYWGQGTSVTVSS

SEQ ID NO: 391 - protein, 123 aa, Artificial Sequence
  Variable Heavy Chain -- TM-18
EVRLQQSGPELVKPGASVKISCKTSGYTFTDYYMNWVKQSHGKSLEWIVNINPNNGDAFYNQKFKGKATLTVDKSSNTAYLDLRSLTSEDSAVYYCAREGQLRLRRVYAMDYWGQGTSVTVSS

SEQ ID NO: 392 - protein, 120 aa, Artificial Sequence
  Variable Heavy Chain -- TM-19
DVQLQQSGAELVRPGASVKLSCTASGFNIKDYYMYWVKQRPEQGLEWIGRIDPEDGDTENAPKFRGMATMTADTSSNTAYLQLNSLTSEDTAVYYCTTRIGNLYHVMDYWGHGTSVTVSS

SEQ ID NO: 393 - protein, 117 aa, Artificial Sequence
  Variable Heavy Chain -- TM-21
EVQLVESGGGLVKPGGSLKLSCAASGFTFSNYAMSWVRETPEKRLEWVAFISDGGGYIYYADNVKDRFTISRDNAKNNLYLQMRHLKSEDTAMYYCARDGGTGFTYWGQGTLVTVSV

SEQ ID NO: 394 - protein, 121 aa, Artificial Sequence
  Variable Heavy Chain -- TM-22
QVQLQQSGPELVKPGASVKISCKASGYRFTSYYIHWVKQRPGQGLEWIGWIYPGNGITNYNEKFKGKATLTADTSSSTAYMQLSSLTSEDSAVYYCASPYYGIRNCYFDVWGTGTTVTVSS

SEQ ID NO: 395 - protein, 120 aa, Artificial Sequence
  Variable Heavy Chain -- TM-23, TM-26
QVQLQQSGAELIRPGTSVKVSCKASGYAFTNYLIEWVKKRPGQGLEWIGVINPGSGITNYNEKFKGKATLTADKSSSTAYMQLSSLTSEDSAVYFCARSDFITTVVADYWGQGTTVTVSS

SEQ ID NO: 396 - protein, 119 aa, Artificial Sequence
  Variable Heavy Chain -- TM-25
EVQLVESGGDLVKPGGSLRLSCAASGFTFSGYGMSWIRQTPDKRLEWVATISSGGRYTVYPDSVKGRFTMSRDNVKNTLYLQMSSLKSEDTALYYCARDNFYSYAMDYWGLGTSVTVSA

SEQ ID NO: 397 - protein, 120 aa, Artificial Sequence
  Variable Heavy Chain -- TM-27
QVQLQQSGAELVRPGTSVKVSCKASGYALTNYLIEWVKQRPGQGLEWIGVINPGSGSTKYNEKFKGKATLTADKSSSTAYMQLSSLTSEDSAVYFCARIIYDHDWYFDVWGTGTTVTVSS

SEQ ID NO: 398 - protein, 118 aa, Artificial Sequence
  Variable Heavy Chain -- TM-28
EVQLQQSGPVLVKPGPSVKISCMASVFTFNDYYIHWVKQSHGKSLEWIGLVYPYNGGTNYNQNFKGKATLTVDTSSRTAYMELNSLTSEDSAVYYCARSYFSNPIGYWGQGTLVTVSE

SEQ ID NO: 399 - protein, 117 aa, Artificial Sequence
  Variable Heavy Chain -- TM-29
QVQLRQSGAELVRPGASVKLSCKASGYTFTDYYINWVKQRPGQGLEWIARIYPGSGYTYYNEKFKGKATLTAEGSSNTAYMQLSSLTSEDSAVYFCANHYTNPFAYWGQGTLVTVSA

SEQ ID NO: 400 - protein, 117 aa, Artificial Sequence
  Variable Heavy Chain -- TM-30
QVQLQQSGPELVKPGASVKLSCKASDNTFTNYDVNWVRQRPGQGLEWIGWIYPRDGTTIYNEKFKGRATLTVDTSSSTAYMELHSLTSEDSAVFFCARTLPQAMDYWGQGTSVTVSS

SEQ ID NO: 401 - protein, 122 aa, Artificial Sequence
  Variable Heavy Chain -- TM-31
QVQLQQSGAELAKPGASVKVSCKASGYTFISYWMNWVKQRPGQGLEWIGYINPTSGYTRYNQKFKDKATLTADKSSSTAYMQLSSLTYEDSAVYYCARSPPTVVLIGYFDYWGQGTTLTVSS

SEQ ID NO: 402 - protein, 120 aa, Artificial Sequence
  Variable Heavy Chain -- TM-32
EVQLQQSGAELVRPGASVRLSCIASGFNIKDYYMYWVKQRPEQGLEWIGRIDPEDGDTEYVPKFQGKATMTADTSSNTAYLQLSSLTSEDIGVYYCTTRTWDLYYAVDNWGQGTSVTVSS

SEQ ID NO: 403 - protein, 111 aa, Artificial Sequence
  Variable Heavy Chain -- TM-33
EFQLQQSGPELVKPGASVKISCKASGYSFTDYNMNWVKQSNGKSLEWIGVINPNYGTTSYNQKFKGKATLTVDQSSSTAYMQLNSLTSEDSAVYYCASSYWGQGTLVTVSA

SEQ ID NO: 404 - protein, 119 aa, Artificial Sequence
  Variable Heavy Chain -- TM-34
EVQLVESGGGLVKPGGSLKLSCAASGFSFSDSGMDWVRQAPEKGLEWFAYISSGSSTTHYADTVKGRFIISRDNAKNTLFLQMTSLRSEDTAMYYCVRRDGNYWYFDVWGTGTTVTVSS

SEQ ID NO: 405 - protein, 120 aa, Artificial Sequence
  Variable Heavy Chain -- TM-35
QVQLQQSGPELVRPGTSVKISCKAPGYTFTRHWMQWVRQRPGQGLEWIGEILPGSNNIYYNEKVKGKATLTVDTSSSTSYMQLSSLTSEDSAVYFCARSLYDYDGVFAYWGQGTLVTVSA

SEQ ID NO: 406 - protein, 121 aa, Artificial Sequence
  Variable Heavy Chain -- TM-37
QVQLQQSGAELAKPGASVKLSCKASGYTFTSYWMHWVKQRPGQGLEWIGYVNPSSGYTKNNQKFKDKVTLTADKSSSTAYMQLSSLTYEDSAVYYCAREGGSISDWYFDVWGTGTTVTVSS

SEQ ID NO: 407 - protein, 121 aa, Artificial Sequence
  Variable Heavy Chain -- TM-39
HVQRQQSGTELAKPGASVKLSCKTSGYTFTDYWMHWIKQRPGQGLEWIGFINPSSGYTKYNQNFKDKATLTADKSSSTAYMQLSSLTYEDSAVFYCAREAGSISDWYFDVWGTGTTVTVSS

SEQ ID NO: 408 - protein, 116 aa, Artificial Sequence
  Variable Heavy Chain -- TM-41
EVQLQQSGAELVKPGASIKLSCTASGFNIKDCYMHWVKQRTEQGLEWIGRIDPEDGTTNFAPKFQDRATITADTSSNTAYLQLTSLTSEDTAVYYCAREWDSGAYWGQGTLVTVSA

SEQ ID NO: 409 - protein, 121 aa, Artificial Sequence
  Variable Heavy Chain -- TM-42, TM-45
QVQLQQSGAELAKPGASVKLSCKASGYTFTNYWMHWVKKRPGQGLEWIGYINPSSGYTKYNQKFKDKATLTADKSSNTAYMQLSSLTYEDSAVYYCTREGGSISDWYFDVWGTGTTVTVSS

SEQ ID NO: 410 - protein, 119 aa, Artificial Sequence
  Variable Heavy Chain -- TM-44
EVQLVESGGGLVKPGGSLKLSCAASGFTFSDYGMHWVRQAPEKGLEWVAYISSGSSTIYYADTVKGRFTISRDNAKNTLFLQMTSLRSEDTAMYYCARNYGSPYAMDYWGQGTSVTVSS

SEQ ID NO: 411 - protein, 118 aa, Artificial Sequence
  Variable Heavy Chain -- TM-46
QVQLQQSGADLVKPGASVKISCKASGYAFSSFWMNWVKLRPGKGLEWIGQIYPGDGDTDYNGKFKDKATLTADTSSNTAYMQLSRLTSEDSAVYFCARGDGFSYFDYWGQGTILTVSS

SEQ ID NO: 412 - protein, 120 aa, Artificial Sequence
  Variable Heavy Chain -- TM-47
QVQLKESGPVLVAPSQSLSITCTVSGFSLTNYGVHWVRQPPGKGLEWLGVIWAGGNTNYNSALMSRLSISKDNSKSQVFLKMNSLQTDDTAMYYCAKEAKLLRSYAMDYWGQGTSVTVSS

SEQ ID NO: 413 - protein, 117 aa, Artificial Sequence
  Variable Heavy Chain -- TM-48, TM-60
QVQLQQSGPELVKPGASVKLSCKASGYTFTSYDINWVKQRPGQGLEWIGWIYPRDGNTQYIEKLKGKATLTVDTSSSTAYMELHSLTSEDSAVYFCARWIFYAMDYWGQGTSVTVSS

SEQ ID NO: 414 - protein, 119 aa, Artificial Sequence
  Variable Heavy Chain -- TM-49
QVQLQQPGAELVRPGSSVKLSCKASGYTFTRFWMHWVKQRPIQGLEWIGNIDPSDSQTHYNQKFKDKATLTVDKSSSTAYMQLSSLTSEDSAVYYCARLITVDYAMDYWGQGTSVTVSS

SEQ ID NO: 415 - protein, 120 aa, Artificial Sequence
  Variable Heavy Chain -- TM-50
QVQLKESGPVLVAPSQSLSITCTVSGFSLTNYGVHWVRQPPGKGLEWLGVIWAGGNTNYNSALMSRLSISKDNSKSQVFLKMNSLQTDDTAMYYCAKEAKLLRSYAMDYWGQGTLVTVSA

SEQ ID NO: 416 - protein, 126 aa, Artificial Sequence
  Variable Heavy Chain -- TM-51
QVQLQQPGAELVKPGASVKLSCKASGYTFTTYWIDWVKQRPGQGLEWIGNMFPGSSRTNYNEKFKSRATLTVDTSSSTAYMQLSSLTSDDSAVYYCARKEGLWTYGYDGGAWFAYWGQGTLVTVSA

SEQ ID NO: 417 - protein, 120 aa, Artificial Sequence
  Variable Heavy Chain -- TM-52
QVQLQQPGAELVKPGASVKLSCKASGYTFTNYWMHWVKQRPGQGLDWIGNIDPSDSETHYNQKFKDKATLTVDKVSSTAYMQLSSLTSEDSAVYYCARRGYYGRSPFAYWGQGTLVTVSA

SEQ ID NO: 418 - protein, 112 aa, Artificial Sequence
  Variable Light Chain -- TM-1
DVLMTQTPLSLPVSLGDHASISCRSSQSIVYNNGNTYLEWYLQKPGQSPKLLIYKVSNRFSGVPDRFSGSGSGTDFTLKISRVEAEDLGVYYCFQVSHVPFTFGSGTKLEIK

SEQ ID NO: 419 - protein, 106 aa, Artificial Sequence
  Variable Light Chain -- TM-2
QIVLTQSPAILSASPGEKVTMTCRASSSVSYIHWFLQKPGSSPKPWIYATSNLASGVPFRFIGSGSGTSYSLTISGVEAEDSATYYCQQWSSNPSTFGAGTKLELK

SEQ ID NO: 420 - protein, 112 aa, Artificial Sequence
  Variable Light Chain -- TM-3
DIVMTQAAFSNPVTLGTSASISCRSSKSLLHSNGITYLYWYLQKPGQSPQLLISQMSSLASGVPDRFSSGGSGTDFTLRISRVEAEDVGVFYCAQNLELPWTFGGGTKLELK

SEQ ID NO: 421 - protein, 109 aa, Artificial Sequence
  Variable Light Chain -- TM-4
QAVVTQESALTTSPGETVTLTCRSSTGAVTTSNYANWVQEKPDHLFTGLIGGTNNRAPGVPARFSGSLIGDKAALTITGAQTEDEAIYFCVLWYSNHLVFGGGTKLTVL

SEQ ID NO: 422 - protein, 107 aa, Artificial Sequence
  Variable Light Chain -- TM-5
DIQMTQSPASLSASVGETVTITCRASENIYIYLAWYQQKQGKTPQLLVYNGKMLAENVPSRFSGSGSGTQFSLKINSLQPEDFGSYYCQHHYGSPPAFGAGTKLELK

SEQ ID NO: 423 - protein, 107 aa, Artificial Sequence
  Variable Light Chain -- TM-6
DIQMSQSSSYLSVSLGGRVTITCKASDHINNWLAWYQQKPGNAPRLLISGATSLETGVPSRFSGSGSGKDYTLSITSLQTEDVATYYCQQYWSSPYTFGGGTKLEIK

SEQ ID NO: 424 - protein, 113 aa, Artificial Sequence
  Variable Light Chain -- TM-7
DIVMTQSPSSLSVSAGEKVTMSCKSSQSLLNSGNQRNYLAWYQQKPGQPPKLLIYGASTRESGVPDRFTGSGSGTDFTLTISNVQAEDLAVYYCQNDHSYPLTFGAGTKLELK

SEQ ID NO: 425 - protein, 107 aa, Artificial Sequence
  Variable Light Chain -- TM-8
DIKMTQSPSSMYASLGERVTITCKASQDINSYLSWFQQKPGKSPKTLIFRANRLVDGVPSRFSGSGSGQDYSLTISSLEYEDMGIYYCLQYDEFPLTFGAGTKLEMK

SEQ ID NO: 426 - protein, 112 aa, Artificial Sequence
  Variable Light Chain -- TM-9
DIVMTQAAFSNPVTLGTSASISCRSSKSLLHSNGITYLYWYLQKPGQSPQLLIYQMSNLASGVPNRFSSSGSGTDFTLRISRVEAEDVGVYYCAQNLELPWTFGGGTKLEIK

SEQ ID NO: 427 - protein, 112 aa, Artificial Sequence
  Variable Light Chain -- TM-10
DVVMTQTPLSLPVSLGDQASISCRSSQSLVHSNGKTYLHWYVQKPGQSPKLLIYKISNRFSGVPDRFSGSGSGTDFTLKISRVEAEDLGVYFCSQITHVPWTFGGGTKLESK

SEQ ID NO: 428 - protein, 113 aa, Artificial Sequence
  Variable Light Chain -- TM-11
DIVMTQSPSSLTMSVGQKVTMHCKSSQSLLNSNNLQNYLAWYQQKPGQSPTLLVYFASIRESGVPDRFIGSGSGTDFTLTISSVQAEDLADYFCQQHYNTPFTFGSGTKLEIR

SEQ ID NO: 429 - protein, 112 aa, Artificial Sequence
  Variable Light Chain -- TM-12
DVLMTQIPLSLPVSLGDQASISCRSSQTIVHSNGNTYLEWYLKKPGQSPKLLIDKISNRFSGVPDRFSGSGSGTDFTLKISRVEAEDLGVYYCFQGSHVPYTFGGGTKLEIK

SEQ ID NO: 430 - protein, 107 aa, Artificial Sequence
  Variable Light Chain -- TM-13
DIQMTQSPASLSVSVGETVTITCRASENIYSSLGWYQQKQGESPQLLVFAATNLADGVPSRFSGSGSGTQYSLKINSLQSEDFGTYYCQHLWSIPWTFGGGTRLEIK

SEQ ID NO: 431 - protein, 107 aa, Artificial Sequence
  Variable Light Chain -- TM-14
DIQMTQSPSSLSASLGERVSLTCRASQDIGSNLNWLQQEPDGTIKRLIYATSSLDSGVPKRFSGSRSGSDYSLTISSLESEDFVVYYCLQYASSPRTFGGGTRLEIK

SEQ ID NO: 432 - protein, 107 aa, Artificial Sequence
  Variable Light Chain -- TM-15
DIQMTQSPASLSVSVGETVTITCRVSENIYNNLAWYQQKQEKSPQLLVFAATNLADGVPSRFSGSGSGTQFSLKINSLQSEDFGTYYCQHFWDTPFTFGSGTKLEIK

SEQ ID NO: 433 - protein, 106 aa, Artificial Sequence
  Variable Light Chain -- TM-16
QIVLTQSPAIMSASPGEKLTMTCSASSSLNYMYWYQQKPGSSPRLLIYDTSNLASGVPVRFSGSGSGTSYSLTISRMEAEDGATYYCQQWTSFPPTFGAGTKLELK

SEQ ID NO: 434 - protein, 107 aa, Artificial Sequence
  Variable Light Chain -- TM-17
DIQMTQSPSSLSASLGERVSLTCRASQDIGSNLNWLQQEPDGTIKRLIYATSSLDSGVPKRFSGSRSGSDYSLTISSLESEDFVDYYCLQYASSPRTFGGGTKLEIK

SEQ ID NO: 435 - protein, 111 aa, Artificial Sequence
  Variable Light Chain -- TM-18
DIVLAQSPASLAVSLGQRATISCRASKSVSISVYTYVHWYQQKPGQPPKLLIYLASNLESGVPARFSGSGSGTDFTLNIHPVEEEDAATYYCQHSRELPYTFGGGTKLEIK

SEQ ID NO: 436 - protein, 112 aa, Artificial Sequence
  Variable Light Chain -- TM-19
DVLMTQTPLSLPVSLGDQASISCRSSQSIVHSNGNTYLEWYLQKPGQAPKLLIDKVFNRFSGVPDRFSGSGSGTDFTLKISRVEAEDLGVYYCFQGSHVPFTFGSGTKLEIK

SEQ ID NO: 437 - protein, 106 aa, Artificial Sequence
  Variable Light Chain -- TM-20
QIVLTQSPAIMSASPGEKLTMTCSASSSLNYMYWYQQKPGSSPRLLIYDTSNLASGVPVRFRGSGSGTSYSLTISRMEAEDGATYYCQQWTSFPPTFGAGTKLELK

SEQ ID NO: 438 - protein, 112 aa, Artificial Sequence
  Variable Light Chain -- TM-21
DIVMTQAAFSNPVTLGTSASISCRSSKSLLHSNGITYLFWYLQKPGQSPQLLIYQMSNLASGVPDRFSSSGSGTDFTLRISRVEAEDVGVYYCVQNLELPYTFGGGTKLEIK

SEQ ID NO: 439 - protein, 112 aa, Artificial Sequence
  Variable Light Chain -- TM-22
DVLMTQTPLSLPVSLGDHASISCRSGQSIVHSNGNTYLEWYLQKPGQSPKLLIYKVSNRFSGVPDRFSGSGSGTDFTLKISRVEAEDLGIYYCFQGSHVPWTFGGGTKLEIK

SEQ ID NO: 440 - protein, 112 aa, Artificial Sequence
  Variable Light Chain -- TM-23, TM-26
DIVVTQTPLSLPVSFGDQVSISCRSSQSLTNYYGNTYLSWYLHKPGQSPQLLIYGISNRFSGVPDRFSGSGSGTDFTLKISTIKPEDLGMYYCLQGTHQPRTFGGGTKLEIK

SEQ ID NO: 441 - protein, 113 aa, Artificial Sequence
  Variable Light Chain -- TM-24
DIVMTQSPSSLTMSVGQKVTMNCKSSQSLLNSNNQQNYLAWYQQKPGQSPKLLVYFASIRESGVPDRFIGSGSGTDFTLTINSVQAEDLADYFCQQHYSTPFTFGSGTKLEIR

SEQ ID NO: 442 - protein, 111 aa, Artificial Sequence
  Variable Light Chain -- TM-25
DIVMTQAAFSNPVTLGTSASISCRSSKSLLHSNGITYLFWYLQKPGQSPQLLIYQMSNLASGVPDRFSSSGSGTDFTLRISRVEAEDVGVYYCAQNLELWTFGGGTKLEIK

SEQ ID NO: 443 - protein, 106 aa, Artificial Sequence
  Variable Light Chain -- TM-27
QIVLTQSPAIMSASPGEKVTISCSASSSISYMYWYQQKPGSSPKPWIYRTSTLASGVPARFSGSGSGTSYSLTISSMEAEDAATYYCQQYHSYPRTFGGGTKLEIK

SEQ ID NO: 444 - protein, 113 aa, Artificial Sequence
  Variable Light Chain -- TM-28
DIVMTQSPSSLSVSVGEKVTVSCKSSQSLLNSGNQKNYLAWYQQKPGQPPKLLIYGASTRESGVPDRFTGSGSGTDFTLTISSVQAEDLAVYYCQNDHSYPLTFGAGTKLELK

SEQ ID NO: 445 - protein, 112 aa, Artificial Sequence
  Variable Light Chain -- TM-29
DIVMTQAAFSNPVTLGTSASISCRSSKSLLHYNGITYLYWYLQKPGQSPQLLIYQMSNLASGVPDRFSSSGSGTDFTLRISRVEAEDVGVYYCAQNLELPYTFGGGTKLEIK

SEQ ID NO: 446 - protein, 112 aa, Artificial Sequence
  Variable Light Chain -- TM-30
DVLMTQSPLSLPVSLGDQVSISCRSSQTIVHRNGNTYLEWYLQKPGQSPKLLIYKVSNRFSGVPDRFSGSGSGTDFTLKISRVEAEDLGVYYCFQGSHLPWTFGGGTKLEIK

SEQ ID NO: 447 - protein, 107 aa, Artificial Sequence
  Variable Light Chain -- TM-31
DIKMTQSPSSMYASLGERVTFTCKASQDINSYLSWFQQKPGKSPKTLIYRGNGLVDGVPSRFSGSGSGQDYSLTISSLEYEDMGIYYCLQYDEFPFTFASGTKLEIK

SEQ ID NO: 448 - protein, 112 aa, Artificial Sequence
  Variable Light Chain -- TM-32
DVLMTQTPLSLPVSLGDQASISCRSSQNIVHSNGNTYLEWYLQKPGQSPKLLIDKVSNRFSGVPDRFSGSGSGTDFTLKISRVEAEDLGIYYCFQGSHVPFTFGSGTKLEIK

SEQ ID NO: 449 - protein, 107 aa, Artificial Sequence
  Variable Light Chain -- TM-33
DIVMTQSQKFMSTTVGDRVSITCKASQNVGTAVAWYQQKPGQSPKLLIYSASNRYTGVPDRFTGSGSGTDFTLTISNMQSEDLADYFCQQYSSYPYTFGGGTKLEIK

SEQ ID NO: 450 - protein, 106 aa, Artificial Sequence
  Variable Light Chain -- TM-34
QIVLSQSPVILSASPGEKVTMTCRATSSVTYMHWYQLKPGSSPKPWIYATSNLASGVPARFSGSGSGTSYSLTISRVEAEDAATYYCQQWSSNPYTFGGGTKLEIK

SEQ ID NO: 451 - protein, 112 aa, Artificial Sequence
  Variable Light Chain -- TM-35
DVLMTQIPLSLPVSLGDQASISCRSSQSIVHRNGNTYLEWYLQKPGQSPKLLIYKVSNRFSGVPDRFSGSGSGTDFTLKISRVEAEDLGVYYCFQGSHVPYTFGGGTKLEIK

SEQ ID NO: 452 - protein, 112 aa, Artificial Sequence
  Variable Light Chain -- TM-37, TM-59
DIVMTQAAPSVPVTPGESVSISCRSSKSLLHSNGNTYSYWFLQRPGQSPQLLIYRMSNLASGVPDRFSGSGSGTAFTLRISRVEAEDVGVYYCMQHLEYPYTFGGGTKLEIK

SEQ ID NO: 453 - protein, 112 aa, Artificial Sequence
  Variable Light Chain -- TM-39
DIVMTQAAPSIPVTPGESVSISCRSSKTLLNSNGNTYLYWFLQRPGQPPQLLIYRMSNLASGVPDRFSGSGSGTAFTLRISRVEAEDVGVYYCMQHLDYPYTFGGGTQLEIK

SEQ ID NO: 454 - protein, 113 aa, Artificial Sequence
  Variable Light Chain -- TM-41
DIVMSQSPSSLAVSVGQKVTMSCKSSQSLLYSSNQKNYLAWYQQKPGQSPKLLIYWASTRESGVPDRFTGSGSGTDFTLTISSVKTEDLAVYYCQQYYSYPYTFGGGTKLEIK

SEQ ID NO: 455 - protein, 112 aa, Artificial Sequence
  Variable Light Chain -- TM-42, TM-45
DIVMTQAAPSLPVTPGESVSISCRSSKSLLHSNGNTYLYWFLQRPGQSPQLLIYRMSNLASGVPDRFSGSGSGTAFTLRISRVEAEDVGVYYCMQHLEYPYTFGGGTKLEIK

SEQ ID NO: 456 - protein, 112 aa, Artificial Sequence
  Variable Light Chain -- TM-44
DIVMSQSPSSLAVSVGEKVTMSCKSSQSLLYSSNQKNYLAWYQQKPGQSPKLLIYWASTRESGVPDRFTGSGSGTDFTLTISSVKAEDLAVYYCQQYYSYPTFGGGTKLEIK

SEQ ID NO: 457 - protein, 106 aa, Artificial Sequence
  Variable Light Chain -- TM-46
QIVLTQSPVIMSASPGERVTMTCCASSRVNYMHWYQQKSGSYPKRWIYDTSKLASGVPGRFSGSGSGTSYSLTISSMEAEDAATYYCQQWSSNPPTFGGGTKLEIK

SEQ ID NO: 458 - protein, 113 aa, Artificial Sequence
  Variable Light Chain -- TM-47
DIVMTQSPSSLTVTAGEKVTMSCKSSQSLLNSGNQKNYLTWYQQKPGQPPKLLIYWASTRESGVPDRFTGSGSGTDFTLTISSVQAEDLAVYYCQNDYSYPLTFGAGTKLEIK

SEQ ID NO: 459 - protein, 112 aa, Artificial Sequence
  Variable Light Chain -- TM-48
DVLMTQTPLSLPVSLGNQASISCRSSQSIVHGNGNTYLEWYLQKPGQSPKLLIYKVSNRFSGVPDRFSGSGSGTDFTLKISRVEAEDLGVYYCFQGSHLPYTFGGGTKLEIK

SEQ ID NO: 460 - protein, 106 aa, Artificial Sequence
  Variable Light Chain -- TM-49
QIVLTQSPAIMSASPGEKVTISCSASSSVSYMYWYQQKPGSSPKPWIHRTSNLASGVPVRFSGSGSGTSYSLTISSMEAEDAATYYCQQYHSYPPTFGAGTKLELK

SEQ ID NO: 461 - protein, 107 aa, Artificial Sequence
  Variable Light Chain -- TM-50
DVQMTQTTSSLSASLGDRVTISCRASQDINNYLYWYQQKPDGTVKLLIYYTSMLHSGVPSRFSGSGSGTDYSLTISNLEQEDIATYFCQQGSTLMYTFGGGTKLEIK

SEQ ID NO: 462 - protein, 112 aa, Artificial Sequence
  Variable Light Chain -- TM-51
DIVMSQSPSSLAVSVGEKVTMSCKSSQSLRNSRTRKNYLAWYQQKPGQSPKLLIYWASTRESGVPDRFTGSGSGTDFTLTISSVQAEDLAIYYCKQSYNLLTFGAGTKLELK

SEQ ID NO: 463 - protein, 106 aa, Artificial Sequence
  Variable Light Chain -- TM-52
ENVLTQSPAIMSASPGEKVTMTCSASSSVSYMHWYQQKSNTSPKLWIYDTSKLASGVPGRFSGSGSGNSYSLTISSAEAEDVATYYCFQGSGYPLTFGAGTKLELK

SEQ ID NO: 464 - protein, 117 aa, Artificial Sequence
  Variable Heavy Chain -- TM-54
QVQMQQSGPVLVKPGASVTLSCKASGYTFTTYDLNWVKQRPGQGLEWIGWIYPRGGSTEYNEKFKGKATLTVDTSSSTAYMELHSLTSEDSAVYFCAKYSIYAMDYWGQGTSVTVSS

SEQ ID NO: 465 - protein, 121 aa, Artificial Sequence
  Variable Heavy Chain -- TM-56
QVHLQQSGAELAKPGASVKLSCKASGYTFTSYWMHWVKQRPGQGLEWIGYINPSSGYSKYNQRFKDKATLTADKSSTTAYMHLSSLTYADSAVYYCTREGGSISDWYFDVWGTGTTVTVSS

SEQ ID NO: 466 - protein, 121 aa, Artificial Sequence
  Variable Heavy Chain -- TM-59
QVQLQQSGAELAKPGASVKLSCKASGYTFISYWMHWVKQRPGQGLEWIGYVNPSSGYTKNNQKFKDKATLTADKSSSTAYMQLSSLTYEDSAVYYCAREGGSISDWYFDVWGTGTTVTVSS

SEQ ID NO: 467 - protein, 121 aa, Artificial Sequence
  Variable Heavy Chain -- TM-61
QVQLQQSGAELAKPGASVKLSCKASGYTFTNYWMNWVKQRPGQGLEWIGYINPSSDYTKYNQNFKDKATLTADKSSSTAYMQLNSLTYEDSAVYYCTRWGGSRSYWYFDVWGAGTTVTVSS

SEQ ID NO: 468 - protein, 119 aa, Artificial Sequence
  Variable Heavy Chain -- TM-62
QVQLQQPGTELVRPGSSVKLSCKAAGYTFTRYWMHWVKQRPIQGLEWIGNIDPSDSETQYNPKFKDKATLTVDRSSSTAYMHLSSLTSEDSAVYYCARLTTLDYAMDYWGQGTSVTVSS

SEQ ID NO: 469 - protein, 118 aa, Artificial Sequence
  Variable Heavy Chain -- TM-63
QVQLQQPGAELVKFGASVKLSCKASGYTFTSFWMHWVKQRPGQGLEWIGNIDPSDSETHYSQKFKDKATLTVDRSSNTAYIQLSSLTSEDSAVYYCARSSNWDNFDYWGQGTTVTVSS

SEQ ID NO: 470 - protein, 123 aa, Artificial Sequence
  Variable Heavy Chain -- TM-64
QVQLQQPGAELVKPGASVKLSCKASGYTFTSYWMHWVKQRPGHGLEWIGNIDPSDSDTHYNQNFRGKATLTVDKFSTTAYMHLSSLTSEDSAVYYCARSRGGTYDYGEAMDYWGQGTSVTVSS

SEQ ID NO: 471 - protein, 115 aa, Artificial Sequence
  Variable Heavy Chain -- TM-65
QVQLQQPGAEFVKPGASVRLSCKTSGYTFTSSWMHWVKQRPGQGLEWIGEINPKNGGTNYNEKFKTKATLTVDKSSNTAYMQLSSLTSEDSAVYYCTIPTWFTYWGQGTLVTVSS

SEQ ID NO: 472 - protein, 118 aa, Artificial Sequence
  Variable Heavy Chain -- TM-66
EVMLVESGGALVEPGGSLKLSCAASGITFSNYAMSWIRQTPEKRLEWVATIGSVGSYTYYPDSVKGRFTISRDNARNTLYLQMSSLRSEDTAMYYCARQEGSSHFDYWGQGTTLTVSS

SEQ ID NO: 473 - protein, 116 aa, Artificial Sequence
  Variable Heavy Chain -- TM-68
EVQLQQSGAELVRPGASVKLSCTASGFNIKDDYMHWVRQRPEQGLEWIGWIDPENGDTEYASKFQGKATMTADTSSNTAYLHLSSLTSEDTAVYYCTKPQTATTCWGQGTLVTVSS

SEQ ID NO: 474 - protein, 117 aa, Artificial Sequence
  Variable Heavy Chain -- TM-69
QVQLKESGPDLVQPSQTLSLTCTVSGFSFTSYGVHWVRQPPGKGLEWVGIMGWDDKKYYNSALKSRLSISRDTSKNQVFLKLSSLQTEDTAMYYCTREAGPYGMDYWGQGTSVTVSS

SEQ ID NO: 475 - protein, 118 aa, Artificial Sequence
  Variable Heavy Chain -- TM-70
EVQLVESGGALVKPGGSLKLSCAASGFTFSNYAMSWVRQTPEKRLEWVATIGYGGSYSYYPDSVKGRFTISRDNAKYTLYLQMSGLRSEDTALYYCARQDSNYEGVWFAYWGQGTLVT

SEQ ID NO: 476 - protein, 121 aa, Artificial Sequence
  Variable Heavy Chain -- TM-71
EVQLVESGGALVKPGGSLKLSCAASGFTFSNYAMSWVRQTPEKRLEWVATIGYGGSYSYYPDSVKGRFTISRDNAKNTLYLQMSSLRSEDTALYYCARQDSNYEGVWFAYWGQGTLVTVSS

SEQ ID NO: 477 - protein, 123 aa, Artificial Sequence
  Variable Heavy Chain -- TM-72
QVQLQQPGAELVKPGTSVKLSCKASGSTFTNYWMHWVMQRPGQGLEWIGNIDPSDSDTHYNQKFKDRAKLTVDKSSSTAYMQLSGLTSEDSAVYYCARSRGGYYDYGEAMDYWGQGTSVTVSS

SEQ ID NO: 478 - protein, 120 aa, Artificial Sequence
  Variable Heavy Chain -- TM-73
QVQLQQPGAELVKPGASVKLSCKASGYTFTSHWMHWVKHRPGQGLEWIGEIDPSDSYTYYNQKFKGKATLTVDKSSSTVYMQLSSLTSEDSAVYYCARNYHGSGPPFAHWGQGTLVTVSS

SEQ ID NO: 479 - protein, 121 aa, Artificial Sequence
  Variable Heavy Chain -- TM-74
EVQLQQSGPELVKPGASVKMSCKASGYTFTSYVIHWVKQKPGQGLEWIGFINPNNDGTKYNEKFRGKATLTSDKSSSTAYMELSSLTSEDSAVYYCARPYYDYEGYTMDYGGQGTSVTVSS

SEQ ID NO: 480 - protein, 119 aa, Artificial Sequence
  Variable Heavy Chain -- TM-75
EVQLQQSGPELVKPGASVKMSCKASGYTFTSYVMHWVKQKPGQGLEWIGYINPYNDGTKYNEKFKGKATLTSDKSSSTAYMELSSLTSEDSAVYYCARGGWFPYAMDYWGQGTTVTVSS

SEQ ID NO: 481 - protein, 120 aa, Artificial Sequence
  Variable Heavy Chain -- TM-76
QVTLKESGPGILQPSQTLSLTCSFSGFSLSTSGMGVSWIRKPSGKGLEWLAHIFWDDDKRYNLFLKSRLTVSKDTSSNQVFLMITSVDTTDTATYYCARMGRQRDYFDYWGQGTSLTVSS

SEQ ID NO: 482 - protein, 115 aa, Artificial Sequence
  Variable Heavy Chain -- TM-77
QIQLQQSGAELVRPGASVKLSCKASGYTFTDYEMHWVKQTPVHGLEWIGVIDPETGGTAYNQMFKGKATLTADKSSSTAYMDLRSLTSEDSAVYYCVKYGIEGYWGQGTTLTVSS

SEQ ID NO: 483 - protein, 123 aa, Artificial Sequence
  Variable Heavy Chain -- TM-78
QVQLQQPGAELVKPGASVKLSCKASGYTFTNHWMHWVRQRPGQGLEWIGNIDPSDSDTHYNHKFKDKATLIVDKSSSTAYLQLSSLTSEDSAVFYCARSRGGYYDYGEAMDYWGQGTSVTVSS

SEQ ID NO: 484 - protein, 121 aa, Artificial Sequence
  Variable Heavy Chain -- TM-79
EVQLQQSGPELVKPGASVKMSCKASGYTFTNYVMHWLKQKPGQGLEWIAHITPHNDGTKYNEKFKGKATLTSDRSSSTAYMELNSLTSEDSAIYYCARSGQFGRGDYFHYWGQGTTVTVSS

SEQ ID NO: 485 - protein, 118 aa, Artificial Sequence
  Variable Heavy Chain -- TM-80
QVQLQQPGAELVKPGASVKLSCKASGYTFTTFWMHWVKQRPGQGLEWIGNIDPSDSETHYNQEFKDKATLTVDRSSNTAYMQLSSLTSEDSAVYYCARSSNWDNFDYWGQGTAVTVSS

SEQ ID NO: 486 - protein, 118 aa, Artificial Sequence
  Variable Heavy Chain -- TM-81
QVQLQQSGAELVKPGASVKLSCKASGYTFTSYWMHWVKQRPGQGLEWIGNIDPSDSEIHYNQKFKDKATLTVDKSSSTAYIQLSSLTSEDSAVYYCARSPAGQILDYWGQGTTVTVSS

SEQ ID NO: 487 - protein, 120 aa, Artificial Sequence
  Variable Heavy Chain -- TM-82
QVQLQQSGAELVRPGTSVRVSCKASGYAFTNYLIEWIKQRPGQGLEWIGVINPGSGITDYSEKFKGKARLTADKSSSTAYMQLSSLTSDDSAVYFCTRSPFITTVVADYWGQGTTVTVSS

SEQ ID NO: 488 - protein, 119 aa, Artificial Sequence
  Variable Heavy Chain -- TM-83
QVQLQQPGTELVKPGALVKLSCKASGYTFTTYWIHWVKQRPGQGLEWIGNINPSNGNTKNNERFKTKATLTVDKSSSTAYMQLSSLTSEDSAVYYCVRGDYYGTSYPFWGQGTLVTVSS

SEQ ID NO: 489 - protein, 117 aa, Artificial Sequence
  Variable Heavy Chain -- TM-84
QVQLQQSGAELVKPGASVKMSCKASGYTFTTFPIEWMKQNHGKSLEWIGNFHPYNDDTKYNEKFKGKATLTVEKSSSTVYLELSRLTSDDSAVYYCARYFYGGMDYWGQGTSVTVSS

SEQ ID NO: 490 - protein, 118 aa, Artificial Sequence
  Variable Heavy Chain -- TM-85
QVQLQQPGAELVRAGTSVKLSCKASGYTFIGYWMHWVKQRPGRGLEWIGRIDPYSGDTRYNEKFKNKATLTVDKPSSTAYMQLSSLTSEDSAVYYCARYGLGHAMDYWGQGTSVTVSS

SEQ ID NO: 491 - protein, 118 aa, Artificial Sequence
  Variable Heavy Chain -- TM-86
EVQLQQSGPVLVKPGASVKMSCKASGYTFTDYYIHWVKQSHGRSLEWIGVISPYNDNTNYNQKFKGKATLTVDKSSSTAYMELNSLTSEDSAVYYCVRTYYSNYFDYWGQGTTLTVSS

SEQ ID NO: 492 - protein, 118 aa, Artificial Sequence
  Variable Heavy Chain -- TM-87
EVQLQQSGPVLVKPGASVKLSCKASGYTFTDYYMNWVKQSHGKSLEWIGVINPYNGATSYNQKFQDKATLTVDKSSSTAYMGLNSLTSEDSAVYYCANGNYDWYFDVWGTGTTVTVSS

SEQ ID NO: 493 - protein, 121 aa, Artificial Sequence
  Variable Heavy Chain -- TM-88
EAQLQQSGPVLVKPGASVKMSCKTSGYTFTDYYINWVKQSHGKSLEWIGVINPYSGATYYTQKFKGKATLTVDKSSSTAYMELNSQTSEDSAVYYCARGGYDYGHWYFDVWGTGTTVTVSS

SEQ ID NO: 494 - protein, 122 aa, Artificial Sequence
  Variable Heavy Chain -- TM-89
QVQLQQSGAELVRPGTSVKVSCKASGYAFTNYLIEWVKQRPGQGLEWIGVINPGSGGTDYNEKFKGKATLTADKSSSTAYMQLSSLTSEDSAVYFCARDPYSKYVHYPMDYWGQGTSVTVSS

SEQ ID NO: 495 - protein, 121 aa, Artificial Sequence
  Variable Heavy Chain -- TM-90
QVHLQQSGPELVKPGASVKLSCKASGYPFTSYDINWVKQRPGQGLEWIGWIYPRDGNAKYNEKFKGKATLTVDTSSSTAYMELHSLTSENSAVYFCARVSDYHGGYGMDYWGQGTSVTVSS

SEQ ID NO: 496 - protein, 118 aa, Artificial Sequence
  Variable Heavy Chain -- TM-91
DVQLQESGPGLVKPSQSLSLTCSVTGYSITSGYYWDWIRQFPGNKLEWMGYISYDGNNNYNPSLKNRISITRDTSKNQFFLKLNSVTTEDTATYYCARAVYSVGFAYWGQGTLVTVSS

SEQ ID NO: 497 - protein, 125 aa, Artificial Sequence
  Variable Heavy Chain -- TM-92
EVQLQQSGPELVKPGASVKIPCKASGYTFTDYTMDWVKQSPGKSLEWIGHINPNDGGTFYNQKFKGKATLTVDKSSSTAYMELRSLTSEDSAVYYCARWVYYYGISYEGGAMDYWGQGTSVTVSS

SEQ ID NO: 498 - protein, 123 aa, Artificial Sequence
  Variable Heavy Chain -- TM-93
QVQLKESGPGLVAPSQSLSITCTVSGFSLSSYGISWVRQPPGKGLEWLGVIWTGGGTNYNSALESRLSITKDNSKSQVFLKMNSLQTDDTARYYCARNPPKIFYDYLMYFDYWGQGTTVTVSS

SEQ ID NO: 499 - protein, 118 aa, Artificial Sequence
  Variable Heavy Chain -- TM-94
EVQLQQSGPELVKPGASVKIPCKASGYTFTDYNMDWVKQSHGKSLEWIGDINPYNGGTLYNQKFKGKATLTVDKSSNTAYMEIRSLTYEDTAVYYCARVGYHGTPDYWGQGTTVTVSS

SEQ ID NO: 500 - protein, 112 aa, Artificial Sequence
  Variable Light Chain -- TM-54
DVLMTQTPLSLPVSLGDQASISCRSSQSIVHGNGNTYLEWYLQKPGQSPKLLIYKVSNRFSGVPDRFSGSGSGTDFTLKISRVEAEDLGVYYCFQGSHVPWTFGGGTKLEMK

SEQ ID NO: 501 - protein, 112 aa, Artificial Sequence
  Variable Light Chain -- TM-56
DIEMTQAAPSVPVTPGESVSISCRSSKSLLQNNGNTYLYWFLQRPGQPPQLLIYRMSNLASGVPDRFSGSGSGTAFTLRISRVEAEDVGVYYCMQHLEYPYTFGGGTKLEIK

SEQ ID NO: 502 - protein, 112 aa, Artificial Sequence
  Variable Light Chain -- TM-60
DVLMTQTPLSLPVSLGDQASISCRSSQSIVHGNGNTYLEWYLQKPGQSPKLLIYKVSNRFSGVPDRFSGSGSGTDFTLKISRVEAEDLGVYYCFQGSHLPYTFGGGTKLEIK

SEQ ID NO: 503 - protein, 113 aa, Artificial Sequence
  Variable Light Chain -- TM-61
DIVMTQSPSSLSASAGEKVTMSCKSSQSLLNSGNQKNYLAWYQQKPGQSPKLLIYGASTRESGVPDRFTGSGSGTDFTLTISSVQAEDLAVYYCQNDSNYPFTFGGGTKLEMK

SEQ ID NO: 504 - protein, 106 aa, Artificial Sequence
  Variable Light Chain -- TM-62
QIVLTQSPAIMSASPGEKVTISCSASSSVSYMYWYQQKAGSSPKPWIHRTSNLASGVPARFSGSGSGTSYSLTIRSMEAEDAATYYCQQYHSYPPTFGVGTKLELK

SEQ ID NO: 505 - protein, 112 aa, Artificial Sequence
  Variable Light Chain -- TM-63
DIVMTQASPSVSVTPGESVSISCRSSQSLLHVNGNTYLYWFLQRPGRSPRLLIYRMSNLASGVPDRFSGSGSGTAFTLRISRVEAEDVGVYYCMQHLQYPFSFGSGTKLEMK

SEQ ID NO: 506 - protein, 112 aa, Artificial Sequence
  Variable Light Chain -- TM-64
DIVMTQAAPSVPVTPGESVSISCRSSKSLLHTNGNTYLFWFLQRPGRSPQLLIYRMSNLASGVPDRFSGSGSGTAFTLRISRVEAEDVGVYYCMQHLEYPFTFGSGTKLEIK

SEQ ID NO: 507 - protein, 107 aa, Artificial Sequence
  Variable Light Chain -- TM-65
DIQMNQSPSSLSASLGDTITITCRASQNINIWLSWYQQKPGNVPKLLIYKASNLHTGVPSRFSGSGSGTGFTLTISSLQPEDIATYYCLQGQSYPYTFGGGTKLGIK

SEQ ID NO: 508 - protein, 112 aa, Artificial Sequence
  Variable Light Chain -- TM-66
DVVVTQTPPSLPVSFGDQVSISCRSSQSLVNSYGKTFLSWYLHKPGQSPQLLIYGISNRFSGVPDRFSGSGSGTVFTLKISTIKPEDLGMYYCLQGTHPPLTFGAGTKLEMK

SEQ ID NO: 509 - protein, 112 aa, Artificial Sequence
  Variable Light Chain -- TM-68
DIVMTQAAFSNPVTLGTSVSISCSSSKSLLHSSGITYLYWYLQRPGQSPQLLIYRMSNLASGVPDRFSGSGSGTDFTLRISRVEAEDVGVYYCAQMLERPWTFGGGTKLEIK

SEQ ID NO: 510 - protein, 112 aa, Artificial Sequence
  Variable Light Chain -- TM-69
DVVMTQTPLSLPVSLGDQASISCRSSQSLVHSNGNTYLHWYLQKPGQSPKLLIYKVSNRFSGVPDRFSGSGSGTDFTLKISRVEAEDLGVYFCSQNTHVPLTFGAGTKLELK

SEQ ID NO: 511 - protein, 111 aa, Artificial Sequence
  Variable Light Chain -- TM-70
DIVLTQSPASLAVSLGQRATISCRASESVDNYGISFMNWFQQKPGQPPKLLIYAASNQGSGVPARFSGSGSGTDFSLNIHPMEEDDTAMYFCQQSKDVPWTFGGGTKLAIK

SEQ ID NO: 512 - protein, 112 aa, Artificial Sequence
  Variable Light Chain -- TM-71, TM-72
DIVMTQAAPSVPVTPGESVSISCRSSKSLLHTNGNTYLFWFIQRPGQSPHLLIYRKSNLASGVPDRFSGSGSGTAFTLRISRVEAEDVGVYYCMQHLEYPFTFGSGTKLEIK

SEQ ID NO: 513 - protein, 106 aa, Artificial Sequence
  Variable Light Chain -- TM-73
QIVLTQSPAIMSASPGEKVTLTCSASSGISYIYWYQQRPGSSPRLLIYDTSNLASGVPVRFSGSGSETSYSLTISRLEAEDAATYYCHQWRSYPPTFGGGTKLEIR

SEQ ID NO: 514 - protein, 107 aa, Artificial Sequence
  Variable Light Chain -- TM-74
DVVMTQSQKFMSTSVEDRVSITCKASQNVRSAVAWYQQKPGQSPKALIYLASNRHTGVPDRFAGSGSGTDFTLTISSVQSEDLADYFCLQHWKYPFTFGSGTKLEIK

SEQ ID NO: 515 - protein, 111 aa, Artificial Sequence
  Variable Light Chain -- TM-75
DIVLTQSPASLAVSLGQRATIFCRASQSVDYNGISYMHWFQQKPGQPPKLLIYAASNLESGIPARFSGSGSGTDFTLNIHPVEEEDAATYYCQQSIEDPWTFGGGTKLEIK

SEQ ID NO: 516 - protein, 105 aa, Artificial Sequence
  Variable Light Chain -- TM-76
QIILTQSPAIMSASLGEEITLTCSASLSISYVHWYQQKSGASPKLLIYGTSNLASGVPSRFSGSGSGTFYSLTISSVEAEDAADYYCHQWSIYRTFGGGTKLEMK

SEQ ID NO: 517 - protein, 112 aa, Artificial Sequence
  Variable Light Chain -- TM-77
DVVMTQIPLTLSVTLGQPASISCKSSQSLFHSNGKTYLNWLLQRPGQSPKLLIYMVSKLESGVPDRFSGSGSGTDFTLKISRVEAEDLGVYYCLQVTHFPLTFGAGTKLELK

SEQ ID NO: 518 - protein, 112 aa, Artificial Sequence
  Variable Light Chain -- TM-78
DIVMTQATPSIPVTPGESVSISCRSSKSLLHVNGNTYLFWFLQRPGQSPQLLIYRMSNLASGVPDRFSGSGSGTAFTLRISRVEAEDVGVYYCMQHLEYPFTFGSGTKLEIK

SEQ ID NO: 519 - protein, 111 aa, Artificial Sequence
  Variable Light Chain -- TM-79
DIVLTQSPASLAVSLGQRATISCRASKSVSTSGSIYIHWYQQKPGQPPKLLIYLTSNLESGVPARFSGSGSGTDFTLNIHPVEEEDAATYYCQHSGELPWTFGGGARLEIK

SEQ ID NO: 520 - protein, 112 aa, Artificial Sequence
  Variable Light Chain -- TM-80
DIVMTQAAPSVPVTPGESVSISCRSSQSLLHVNGHTYLYWFLQRPGQSPQLLIYRMSNLASGVPDRFSGSGSGTVFTLRISRVETEDVGIYYCMQHLQYPFTFGSGTKLEIK

SEQ ID NO: 521 - protein, 113 aa, Artificial Sequence
  Variable Light Chain -- TM-81
DIVMTQSPSSLAMSVGQKVTMSCKSSQSLLNSKNQKNYLAWYQQKPGQSPKFLVYFAFFRESGVPDRFIGSGSGTDFTLTISSVQAEDLADYFCQQHYSTPFTFGSGTKLEIK

SEQ ID NO: 522 - protein, 112 aa, Artificial Sequence
  Variable Light Chain -- TM-82
DVVVTQTPLSLPVSFGDQVSISCRSSQSLANTYGNTYLSWYLHTPGQSPQLLIFGISNRFSGVPDRFSGSGSGTDFTLKISTIKPEDLGVYYCLQGTHPPRTFGGGTKLEMK

SEQ ID NO: 523 - protein, 112 aa, Artificial Sequence
  Variable Light Chain -- TM-83
DVVMTQTPLSLPVSLGDQASISCRSSQSLVHSNGITYLHWYLQKPGQSPKLLIYKVSNRFSGVPDRFSGSGSGTDFTLKISRVEAEDLGVYFCSQSTHVPYTFGGGTKLEIK

SEQ ID NO: 524 - protein, 107 aa, Artificial Sequence
  Variable Light Chain -- TM-84
DIQMTQSPASLSVSVGETVTITCRASENIYSSLGWYHQKQGKSPQLLVFAATNLADGVPSRFSGSGSGTQYSLKINSLQSEDFGTYYCQHLWSVPWTFGGGTKLEIK

SEQ ID NO: 525 - protein, 107 aa, Artificial Sequence
  Variable Light Chain -- TM-85
DIAMSQSQKFMSTSVGDRVSVTCKASQNVGTNVAWYQQKPGQSPKALIYSASYRYSGVPDRFTGSGSGTDFTLTISNVQSEDLAEYFCQQYNSYPLTFGAGTKLEMK

SEQ ID NO: 526 - protein, 113 aa, Artificial Sequence
  Variable Light Chain -- TM-86
DIVMTQSPSSLSVSAGEKVTMSCKSSQSLLNSGNQKNYLAWHQQRPGQPPKLLIYGASTRESGVPDRFTGSGSGTEFTLTISSVQAEDLAVYYCQNDHSYPLTFGAGTKLELK

SEQ ID NO: 527 - protein, 107 aa, Artificial Sequence
  Variable Light Chain -- TM-87
DVQITQSPSYLAASPGETITINCRASRSISKYLAWYQEKPGKTNKLLIYSGSTLQSGIPSRFSGSGSGTDFTLTISSLEPEDFAVYYCQQHNEYPLTFGAGTKLELK

SEQ ID NO: 528 - protein, 107 aa, Artificial Sequence
  Variable Light Chain -- TM-88
ETTVTQSPASLSMAIGEKVTIRCITSSDIDDHMNWYRQKPGEPPEFLISEGNALRPGVPSRFSSSGYGTDFIFTIENILSEDVADYYCLQSDNLPLTFGAGTKLELK

SEQ ID NO: 529 - protein, 107 aa, Artificial Sequence
  Variable Light Chain -- TM-89
DIQMTQSPSSLSASLGERVSLTCRASQDIGSSLNWLQQEPDGTIKRLIYATSSLDSGVPKRFSGSRSGSDYSLTISSLESEDFVDYYCLQYASSPRTFGGGTKLEIK

SEQ ID NO: 530 - protein, 112 aa, Artificial Sequence
  Variable Light Chain -- TM-90
DVLMTQTPLSLPVSLGDQASISCRSSQSFVHGNGNTYLEWYLQKPGQSPKLLIYKVSHRFSGVPDRFSGSGSGTDFTLKITRVEAEDLGVYYCFQNSHVPHTFGGGTKLEIK

SEQ ID NO: 531 - protein, 107 aa, Artificial Sequence
  Variable Light Chain -- TM-91
DIVMTQSQKFMSTSVGDRVSVTCKASQNVYTNVAWYQQKPGQSPKPLIYSASYRYSGVPDRFTGSGSGTDFTLTISNVQSEDLAEYFCQQYNSYPLTFGGGTKLEIK

SEQ ID NO: 532 - protein, 107 aa, Artificial Sequence
  Variable Light Chain -- TM-92
DVQITQSPSYLAASPGETITINCRASKSISKYLAWYQEKPGKTNKLLIYSGSTLQSGIPSRFSGSGSGTDFTLTISSLEPEDFAMYYCQQHNEYPWTFGGGTKLEIK

SEQ ID NO: 533 - protein, 107 aa, Artificial Sequence
  Variable Light Chain -- TM-93
DIVMTQSHKLMSTSVGDRVSITCKASQDVGSAVAWYQQKPGQSPQLLIYWSSTRLPGVPDRFTGSGSGTDFTLTISNVQSEVLADYFCQQYTSYPLTFGGGTKLEIK

SEQ ID NO: 534 - protein, 111 aa, Artificial Sequence
  Variable Light Chain -- TM-94
DVVMTQTPLSLPVSLGDQASISCRSSQSLVYSNGNTYLHWYLQRPGQSPKLLIYKVSNRFSGVPDRFSGSGSGTDFTLKISRVEAEDLGVYFCSQSTHVYTFGGGTKLEIK